(19) United States
(12) Patent Application Publication
(10) Pub. No.: US 2008/0108144 A1
(43) Pub. Date: May 8, 2008

(54) Method for the Rapid Analysis of Polypeptides

(76) Inventors: Muhammad A. Alam, Victoria (AU); Donald K. Bowden, Victoria (AU); Reinhard I. Boysen, Victoria (AU); Milton T.W. Hearn, Balwyn (AU)

Correspondence Address: DUNLAP CODDING & ROGERS, P.C., PO BOX 16370, OKLAHOMA CITY, OK, 73113, US

(21) Appl. No.: 11/597,761

(22) PCT Filed: May 27, 2005
(86) PCT No.: PCT/AU05/00755

§ 371 Date: Aug. 22, 2007

(30) Foreign Application Priority Data
May 27, 2004 (AU) 2004902922
Jun. 3, 2004 (AU) 2004903001

(51) Int. Cl. G01N 33/72; G01N 33/53

(52) U.S. Cl. 436/66; 435/7.1

(57) ABSTRACT

The invention provides improved sample preparation techniques as will as improved methods of analysis of samples. The techniques include a method of preparing a sample of MALDI-TOF analysis comprising applying a material having a liquid component to a carrier, removing at least a portion of the liquid component, and applying a MALDI matrix over the material to be analysed. In other embodiments, the sample preparation techniques include digestion of peptides prior to analysis by MALDI-TOF, which may be done in the presence of a surfactant, and sandwiching a sample for analysis between layers of MALDI matrix on a sample carrier.

### FIELD OF THE INVENTION

The present invention generally relates to improvements in the area of sample analysis particularly the analysis of samples that contain polypeptides. The invention provides improved sample preparation techniques as well as improved methods of analysis of samples. The improved techniques find particular application in the area of detecting the presence of polypeptides and polypeptide variants within a material. In a particularly preferred embodiment the invention relates to the detection of polypeptide variants by MALDI ToF mass spectrometry. The detection of polypeptide variants is of importance as the presence of polypeptide variants may be indicative of the presence of genetic abnormalities and/or the presence of other undesirable medical conditions.

### BACKGROUND

The ability to accurately analyse materials for the presence of components such as polypeptides is an area growing in importance since the completion of the human genome project. Now that the genetic sequences have been provided it is increasingly important to be able to determine the components of materials in order to provide further information of interest on the material or the organism from which it was sourced. There is therefore an increasing need to provide improved methods of sample analysis of materials that contain components such as polypeptides. This analysis can provide information on the identity of polypeptides and polypeptide variants within the material. This information can be helpful in the diagnosis of certain medical conditions or the characterisation of mutant proteins.

Polypeptides are encoded by DNA and play important roles in most biological functions within organisms. The function performed by a polypeptide is determined by its structure, wherein the specific structure of the polypeptide allows specific interactions to occur with other molecules. The structure of a polypeptide is determined by the interaction of the amino acid side chains of the polypeptide with each other. Thus the overall structure, and hence the specificity, of a polypeptide is ultimately determined by its amino acid sequence.

As the amino acid sequence of a polypeptide is determined by the nucleotide sequence of its corresponding gene, mutations in genes can manifest themselves as variant polypeptides. Variant polypeptides may have altered function and this altered function may result in a clinical condition. Other variant polypeptides may find application in industry where a process may be improved or made more efficient by the presence of the variant. For example fermentation processes may be made more efficient following a mutation in a gene encoding a protein important for the process in question. Characterisation of that mutation may identify useful sites for additional or alternative mutations to further improve the process.

In addition there are numerous clinical examples of genetic mutation causing the expression of variant polypeptides with altered function. For example, many cancers have mutations in the p53 gene. Altered p53 function can dramatically affect a cell's ability to detect and eliminate genetic mutations, thus leaving an individual susceptible to cancer. There are many other examples, such as haemoglobinopathies where mutations within haemoglobin genes may result in clinical conditions such as α-thalassaemia. Sickle cell-anaemia, for example, results from a single point mutation in the gene encoding β-globin whereby the Glu-6(β) residue in Hb A is replaced by Val in sickle Hb (Hb S). It is thought that this hydrophobic side chain initiates a process by which the densely packed deoxyhaemoglobin tetramers inside the red cells interact with other side chains to form long polymeric fibres that distort the cells into a characteristic sickle shape. At least in theory if rapid analytical techniques could be developed these could be used in the diagnosis of disease states at an early stage allowing for early intervention strategies to be implemented.

Unfortunately many of the known analytical techniques used to analyse polypeptides are either not amenable to high throughput analysis or are such that they do not provide the required sensitivity to accurately distinguish between closely related polypeptides. As will be appreciated the ability to effectively distinguish between two closely related polypeptides is crucial. Without this ability any analytical technique is only capable of providing gross data on the polypeptides in the material studied. In addition many of the techniques are not sufficiently sensitive to be able to identify the presence of small amounts of polypeptide in very complex samples. This thus limits their usefulness.

Thus there remains a need for improved methods of analysing polypeptides to be developed, preferably ones which may be applicable in a clinical setting. Following significant research the present applicants identified MALDI-TOF mass spectrometry (MS) analysis as a diagnostic tool that showed promise. The present invention provides novel, rapid procedures utilising MALDI-TOF MS for analyzing polypeptides directly from a very small quantity of material. Thus, specific embodiments of the present invention provide methods useful for the clinical diagnosis of haemoglobinopathies as well as other diseases involving variant polypeptides.

In developing the improved methods the applicants also developed improved sample preparation techniques that were generally applicable to MALDI-TOF MS analysis of any material as well as being applicable to the improved methods and which provided improved outcomes. These improved sample preparation techniques typically provided improved sensitivity and sample to sample reproductity.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

### SUMMARY OF THE INVENTION

As noted above the present invention relates to a number of improvements in relation to sample preparation techniques for MALDI-ToF MS analysis and the use of these sample preparation techniques in the analysis of polypeptides.

In a first aspect, the present invention provides a method of preparing a sample for MALDI-TOF MS analysis including the steps of:
a) applying a material to be analysed to a carrier, the material to be analysed including a liquid component,
b) removing at least a portion of the liquid component,
c) applying a MALDI matrix over the material to be analysed.

The material to be analysed preferably includes a biological material or is derived from a biological material. Any biological material may be used including blood, cerebrospinal fluid, urine, saliva, seminal fluid or sweat or a combination thereof. It is preferred that the biological material is blood or derived from blood. Preferably the biological material includes a polypeptide. More preferably the polypeptide is a haemoglobin polypeptide or a fragment or variant or a haemoglobin peptide containing a covalently bonded adduct thereof. Preferably the haemoglobin polypeptide may include one or more of the following haemoglobins: α, β, γ, δ, ε or ζ. The biological material is obtained using techniques known in the art. The material may be applied to the carrier in any suitable form by techniques well known in the art. It is preferred that it is applied by a “spotting” technique. It is preferred that the biological material is diluted with a liquid preferably water prior to application. The liquid preferably contains a buffer such as ammonium bicarbonate buffer. The level of dilution will depend on the application but it is preferred that the dilution is from 1:10 to 1:10000. The amount of material applied is typically of the order of 0.1 to 1 0 μl, more preferably 0.5 to 5 μl, most preferably about 1 μl.

Following application of the material to be analysed at least a portion of the liquid component is removed. The liquid component may be removed in any suitable manner that does not destroy the integrity of compounds such as polypeptides within the material. For example the liquid may be removed by subjecting the applied material to elevated temperature, reduced pressure or a combination thereof. The liquid may also be removed by passing a stream of gas (preferably air) over the surface of the applied material. In a particularly preferred embodiment the liquid is removed by allowing the applied material to sit at ambient temperature and pressure for a sufficient time for the liquid to be removed by evaporation.

The amount of liquid removed may vary. It is preferred that at least 50% of the liquid component is removed, more preferably at least 75% of the liquid component is removed, yet even more preferably at least 90% of the liquid component is removed. In another preferred embodiment removal of the liquid component continues until the material is substantially dry, more preferably removal continues until the material is dry. Without wishing to be bound by theory it is felt that adequate removal of the liquid is important to minimise mixing between the material and the latter applied MALDI matrix layer. It is found that mixing of this type reduces the sensitivity of the later analysis.

Following the liquid removal step a MALDI matrix is applied using conventional techniques. Any suitable MALDI matrix may be used however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxy phenylazo)benzoic acid (HABA), succinic acid, 2,6-Dihydroxyacetophenone, Ferulic acid, caffeic acid, 2,4,6-trihydroxyacetophenone (THAP) and 3-hydroxypicolinic acid (HPA), Anthranilic acid, Nicotinic acid, Salicylamide and mixtures thereof. The amount of applied matrix may vary although it is typically of the order such that the ratio of matrix to material to be analysed is from 0.1:1 to 10:1, preferably from 0.5:1 to 5:1, most preferably 1:1 to 2:1.

The material to be analysed is preferably treated to partially digest polypeptides in the material. The digestion may be carried out in solution prior to application to a carrier or may be carried out after the material has been applied to the carrier. In one particularly preferred embodiment the material to be analysed is treated to partially digest polypeptides within the material prior to applying the material to the carrier. In this embodiment it is preferred that the digestion is carried out for from 1 to 24 hours, more preferably 4 to 24 hours. The treatment preferably includes contacting the material with a proteolytic agent. In another preferred embodiment the step of treating the material to partially digest polypeptides in the material is carried out on the carrier and preferably involves contacting the material to be analysed with a proteolytic agent. This may be achieved by addition of a proteolytic agent to the material after it has been applied to the carrier or by addition of a proteolytic agent to the carrier prior to addition of the material. The method preferably includes applying a proteolytic agent to the carrier prior to application of the material to be analysed such that following addition of the material the agent partially digests polypeptides within the material. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used however it is preferred that the proteolytic agent is a protease, preferably a protease selected from the group consisting of trypsin and endoprotease Glu C. In one preferred embodiment the material is treated with a proteolytic agent in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed. This can be readily determined by a skilled worker in the area. The digestion may be stopped in any way well known in the art. For example the digestion may be stopped by addition of a diluted acid. An example of a suitable acid is TFA.

In a second aspect, the present invention provides a method of preparing a sample for MALDI-ToF MS analysis, said sample including a material to be analysed and a carrier, the method including the step of conducting an on carrier digestion of polypeptides within the material.

The material to be analysed preferably includes a biological material or is derived from a biological material. Any biological material may be used in this aspect of the invention including blood, cerebrospinal fluid, urine, saliva, seminal fluid or sweat or a combination thereof. It is preferred that the biological material is blood. Preferably the biological material includes a polypeptide. More preferably the polypeptide is a haemoglobin polypeptide or a fragment or variant or a haemoglobin peptide containing a covalently bonded adduct thereof. Preferably the haemoglobin polypeptide may include one or more of the following haemoglobins: α, β, γ, δ, ε or ζ. The biological material is obtained using techniques well known in the art. The material may be applied to the carrier in any suitable form by techniques well known in the art. It is preferred that the material is applied by a spotting technique. It is preferred that the material is diluted with a liquid, preferably water, prior to applying it to the carrier. The liquid preferably contains a buffer such as ammonium bicarbonate. The level of dilution will depend on the application but it is preferred that the dilution is from 1:10 to 1:10000. The amount of material applied is typically of the order of 0.1 to 10 μl, more preferably 0.5 to 5.0 μl, most preferably about 1 μl. The method includes an on-carrier digest. The on-carrier digest preferably involves contacting the material with a proteolytic agent. This may be achieved by addition of a proteolytic agent to the carrier either prior to, simultaneously with, or following the addition of the material to be analysed.

The method preferably includes application of a proteolytic agent to the carrier prior to application of the material to be analysed such that following addition of the material to be analysed the agent partially digests polypeptides within the material. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used however it is preferred that the proteolytic agent is a protease, preferably a protease selected from the group consisting of trypsin and endoprotease Glu C. In one preferred embodiment the material is treated with a proteolytic agent in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed. The digestion may be stopped in any way well known in the art. For example the digestion may be stopped by addition of a diluted acid. An example of a suitable acid is TFA.

A particularly preferred way of terminating the digestion is by applying a MALDI matrix over the material. Any suitable MALDI matrix may be used however the MALDI matrix is preferably selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxy acetophenone (THAP) and 3-hydroxypicolinic acid (HPA), Anthranilic acid, Nicotinic acid, Salicylamide or mixtures thereof. The amount of applied matrix may vary although it is typically of the order such that the ratio of matrix to sample is from 0.1:1 to 10:1, preferably 0.5:1 to 5:1, most preferably 1:1 to 2:1.

In a third aspect, the present invention provides a sample for analysis having,

(a) a carrier having a surface;
(b) a layer including a material to be analysed, and
(c) a single MALDI matrix layer,
wherein the layer including the material to be analysed is located between the carrier surface and the MALDI matrix layer.

The material to be analysed preferably includes a biological material or is derived from a biological material. Any biological materials may be used including blood, cerebrospinal fluid, urine, saliva, seminal fluid or sweat or a combination thereof. It is preferred that the biological material is blood. Preferably the biological material includes a polypeptide. More preferably the polypeptide is a haemoglobin polypeptide or a fragment or variant or a haemoglobin peptide containing a covalently bonded adduct thereof. Preferably the haemoglobin polypeptide may include one or more of the following haemoglobins: α, β, γ, δ, ε or ζ. It is particularly preferred that the material to be analysed contains partially digested polypeptides.

Any suitable-MALDI matrix may be utilised however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxyacetophenone (THAP) and 3-hydroxypicolinic acid (HPA), Anthranilic acid, Nicotinic acid, Salicylamide and mixtures thereof. It is preferred that the sample has been produced using the methods of the invention described herein.

In a fourth aspect, the present invention provides a method of improving digestion of polypeptides within a material said method including the step of conducting the digestion in the presence of sodium 3-[(2-methyl-2-undecyl-1,3-dioxolan-4-yl)methoxy]-1-propane-sulfonate or a derivative thereof. In a preferred embodiment the digestion includes digestion by proteolytic enzymes.

In a fifth aspect the invention provides a method of analysing a polypeptide including the steps of:
(a) partially digesting the polypeptide,
(b) subjecting the digested polypeptide to MALDI-TOF MS analysis to identify digestion fragments characteristic of the polypeptide.

The step of partially digesting the polypeptide is preferably carried out by contacting the polypeptide with a proteolytic agent. The digestion may be carried out in solution prior to application to a carrier or may be carried out after the material has been applied to the carrier. Accordingly, the polypeptide may be digested either in solution or whilst on a carrier. In one preferred embodiment the digestion is carried out in solution by addition of a proteolytic agent to a solution containing the polypeptide. In this embodiment it is preferred that the digestion is carried out for from 1 to 24 hours, preferably from 4 to 24 hours. Following digestion the material is typically applied to the carrier. The amount of material applied is typically of the order of 0.1 to 10 μl, more preferably 0.5 to 5 μl, most preferably about 1 μl.

Following application of the material to be analysed at least a portion of the liquid component is removed. The liquid component may be removed in any suitable manner that does not destroy the integrity of compounds such as polypeptides within the material. For example the liquid may be removed by subjecting the applied material to elevated temperature, reduced pressure or a combination thereof. The liquid may also be removed by passing a stream of gas (preferably air) over the surface of the applied material. In a particularly preferred embodiment the liquid is removed by allowing the applied material to sit at ambient temperature and pressure for a sufficient time for the liquid to be removed by evaporation.

The amount of liquid removed may vary. It is preferred that at least 50% of the liquid component is removed, more preferably at least 75% of the liquid component is removed, yet even more preferably at least 90% of the liquid component is removed. In another preferred embodiment removal of the liquid component continues until the material is substantially dry, more preferably removal continues until the material is dry. Without wishing to be bound by theory it is felt that adequate removal of the liquid is important to minimise mixing between the material and the latter applied MALDI matrix layer. It is found that mixing of this type reduces the sensitivity of the later analysis.

Following the liquid removal step a MALDI matrix is applied using conventional techniques. Any suitable MALDI matrix may be used however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxy phenylazo)benzoic acid (HABA), succinic acid, 2,6-Dihydroxyacetophenone, Ferulic acid, caffeic acid, 2,4,6-trihydroxyacetophenone (THAP) and 3-hydroxypicolinic acid (HPA), Anthranilic acid, Nicotinic acid, Salicylamide and mixtures thereof. The amount of applied matrix may vary although it is typically of the order such that the ratio of matrix to material to be analysed is from 0.1:1 to 10:1, preferably from 0.5:1 to 5:1, most preferably 1:1 to 2:1.

In another preferred embodiment the digestion is carried out on a carrier. In this embodiment the method preferably includes applying a proteolytic agent to a carrier prior to application of the polypeptide to the carrier such that following addition of the material the agent partially digests the polypeptide. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used however it is preferred that the proteolytic agent is a protease, preferably a protease selected from the group consisting of trypsin and endoprotease Glu C. It is preferred that the material is treated with a proteolytic agent in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed. The digestion may be stopped in any way well known in the art. For example the digestion may be stopped by addition of an acid. An example of a suitable acid is TFA. A particularly preferred way of terminating the digestion of the on carrier digest is by applying a MALDI matrix over the material. Any suitable MALDI matrix may be used however the MALDI matrix is preferably selected from the group consisting of sinapinic acid (SA), α-cyano-4-hydroxycinnamic acid (CHCA), 2,5-dihydroxybenzoic acid (2,5-DHB), 2-(4-hydroxyphenylazo) benzoic acid (HABA), succinic acid, 2,6-Dihydroxyacetophenone, Ferulic acid, caffeic acid, 2,4,6-trihydroxy acetophenone (THAP) and 3-hydroxypicolinic acid (HPA), Anthranilic acid, Nicotinic acid, Salicylamide or mixtures thereof.

The analysis of the MALDI-ToF MS output is conducted in any way well known in the art. It is preferred, however, that the analysis is such that a sequence window is chosen to ensure that fragments exist which cover the entire sequence of the polypeptide. Analysis of this window can then be used to determine digestion fragments characteristic of the polypeptide. Fragments of this type are effectively “signature” fragments and may be indicative of the presence of the polypeptide in a complex mixture that has been digested in a similar manner. The data obtained from such analysis can be added to a database or library of fragments for use in the later identification of the presence of the polypeptide in complex mixtures.

In yet an even further aspect the invention provides a method of determining the identity of one or more polypeptide(s) in a material including the steps of:
(a) partially digesting the material;
(b) analysing the digested material by MALDI-TOF MS to determine digestion fragments,
(c) comparing the digestion fragments with known polypeptide digestion fragments to determine the identity of the polypeptide(s) present.

The material preferably includes a biological material or is derived from a biological material. A number of biological materials may be used including blood, cerebrospinal fluid, urine, saliva, seminal fluid or sweat or a combination thereof. It is preferred that the biological material is blood. Preferably the biological material includes a polypeptide. More preferably the polypeptide is a haemoglobin polypeptide or a fragment or variant or a haemoglobin peptide containing a covalently bonded adduct thereof. Preferably the haemoglobin polypeptide may include one or more of the following haemoglobins: α, β, γ, δ, ε or ζ. It is particularly preferred that the material to be analysed contains partially digested polypeptides.

The step of partially digesting the material preferably involves contacting the material with a proteolytic agent. The digestion may be carried out in solution prior to application to a carrier or may be carried out after the material has been applied to the carrier. Accordingly, the material may be digested either in solution or whilst on a carrier. In one preferred embodiment the digestion is carried out in solution by addition of a proteolytic agent to a solution containing the material. In this embodiment it is preferred that the digestion is carried out for from 1 to 24 hours, more preferably 4 to 24 hours. Any suitable proteolytic agent may be used in the digestion however it is preferred that the proteolytic agent is a protease, preferably a protease selected from the group consisting of trypsin and endoprotease Glu C. In one preferred embodiment the digestion is conducted in the presence of a surfactant. The surfactant is preferably sodium 3-[(2-methyl-2-undecyl-1,3-dioxolan-4-yl)methoxy]-1-propane-sulfonate. The digestion may be stopped by any method well known in the art. Following the in solution digestion the digested material is preferably applied to a carrier.

In this embodiment following application of the material at least a portion of the liquid component is removed. The liquid component may be removed in any suitable manner that does not destroy the integrity of polypeptides or polypeptide fragments within the material. For example the liquid may be removed by subjecting the applied material to elevated temperature, reduced pressure or a combination thereof. The liquid may also be removed by passing a stream of gas (preferably air) over the surface of the applied material. In a particularly preferred embodiment the liquid is removed by allowing the applied material to sit at ambient temperature and pressure for a sufficient time for the liquid to be removed by evaporation.

The amount of liquid removed may vary. It is preferred that at least 50% of the liquid component is removed, more preferably at least 75% of the liquid component is removed, yet even more preferably at least 90% of the liquid component is removed. In another preferred embodiment removal of the liquid component continues until the material is substantially dry, more preferably removal continues until the material is dry. Following the liquid removal step a MALDI matrix is applied. Any suitable MALDI matrix may be used however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxy acetophenone (THAP) and 3-hydroxypicolinic acid (HPA), Anthranilic acid, Nicotinic acid, Salicylamide and mixtures thereof. The amount of MALDI matrix may vary being typically of the order such that the ratio of matrix to added sample is from 0.1 to 1 to 10:1, preferably 0.5:1 to 5:1, most preferably from 1:1 to 2:1.

In another preferred embodiment the digestion is carried out on a carrier. This may be carried out by applying a proteolytic agent either prior to, simultaneously with, or after the application of the material to be analysed. In this embodiment the method preferably includes applying a proteolytic agent to a carrier prior to application of the material to the carrier such that following addition of the material the agent partially digests any polypeptides within the material. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used in the digestion however it is preferred that the proteolytic agent is a protease, preferably a protease selected from the group consisting of trypsin and endoprotease Glu C. In one preferred embodiment digestion occurs in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed for. The digestion may be stopped in any way well known in the art. For example the digestion may be stopped by addition of a diluted acid either to the digestion in solution or to the on carrier digestion. An example of a suitable acid is TFA. A particularly preferred way of terminating the on carrier digestion is by applying a MALDI matrix over the material. Any suitable MALDI matrix may be used however the MALDI matrix is preferably selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxyacetophenone (THAP) and 3-hydroxypicolinic acid (HPA), Anthranilic acid, Nicotinic acid, Salicylamide or mixtures thereof.

Following the production of the sample by the methods described above it is then subjected to analysis by MALDI-TOF MS to determine digestion fragments for the material. The digestion fragments are typically indicative of the polypeptides in the original material. Once the digestion fragments have been determined they are compared to the known digestion fragments (typically called the signature fragments) of known polypeptides. The comparison of the digestion fragments with known digestion fragments or with “signature” digestion fragments of known polypeptides may be carried out in any of a number of ways. For example this can be done manually by scanning the output of the MALDI-TOF MS and comparing it to known digestion fragments to determine the identity of one or more of the polypeptides present. It is preferred that the comparison is carried out by computerised means. In a particularly preferred embodiment the output of the MALDI-TOF MS analysis is compared by computer means to a library of signature fragments to identify a plurality of polypeptides in the material.

In a particularly preferred embodiment the method is used to determine the presence of a polypeptide in a sample. In this embodiment the digestion fragments are compared with the “signature” digestion fragments of the polypeptide of interest to determine if that particular polypeptide is present. This method therefore allows for the determination of the presence of a polypeptide of interest in a complex mixture of polypeptides.

In yet an even further aspect the invention provides a method of analysing a polypeptide variant including the steps of:
(a) partially digesting a material containing the polypeptide variant,
(b) analysing the digested material by MALDI-TOF MS to determine digestion fragments,
(c) comparing the digestion fragments with the digestion fragments of non-variant polypeptides to identify the fragment containing the variation.

The material preferably includes a biological material or is derived from a biological material. Any biological materials may be used including blood, cerebrospinal fluid, urine, saliva, seminal fluid or sweat or a combination thereof. It is preferred that the biological material is blood. Preferably the biological material includes a polypeptide. More preferably the polypeptide is a haemoglobin polypeptide or a fragment or variant or a haemoglobin peptide containing a covalently bonded adduct thereof. Preferably the haemoglobin polypeptide may include one or more of the following haemoglobins: α, β, γ, δ, ε or ζ. It is particularly preferred that the material to be analysed contains partially digested polypeptides.

The digestion preferably involves contacting the material with a proteolytic agent. The digestion may be carried out in solution prior to application to a carrier or may be carried out after the material has been applied to the carrier. Accordingly, the material may be digested either in solution prior to application to the carrier or whilst on a carrier. In one preferred embodiment the digestion is carried out in solution by addition of a proteolytic agent to a solution containing the material. In this embodiment it is preferred that the digestion is carried out for from 1 to 24 hours, more preferably from 4 to 24 hours. Any suitable proteolytic agent may be used in the digestion however it is preferred that the proteolytic agent is a protease, preferably a protease selected from the group consisting of trypsin and endoprotease Glu C. In one preferred embodiment the digestion is carried out in the presence of a surfactant. The surfactant is preferably sodium 3-[(2-methyl-2-undecyl-1,3-dioxolan-4-yl)methoxy]-1-propane-sulfonate. The digestion may be stopped by any method well known in the art. In this embodiment following the in solution digestion the digested material is preferably added to a carrier.

Following application of the material to the carrier at least a portion of the liquid component is removed. The liquid component may be removed in any suitable manner that does not destroy the integrity of polypeptides or polypeptide fragments within the material. For example the liquid may be removed by subjecting the applied material to elevated temperature, reduced pressure or a combination thereof. The liquid may also be removed by passing a stream of gas (preferably air) over the surface of the applied material. In a particularly preferred embodiment the liquid is removed by allowing the applied material to sit at ambient temperature and pressure for a sufficient time for the liquid to be removed by evaporation.

The amount of liquid removed may vary. It is preferred that at least 50% of the liquid component is removed, more preferably at least 75% of the liquid component is removed, yet even more preferably at least 90% of the liquid component is removed. In another preferred embodiment removal of the liquid component continues until the material is substantially dry, more preferably removal continues until the material is dry. Following the liquid removal step a MALDI matrix is applied. Any suitable MALDI matrix may be used however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxy acetophenone (THAP) and 3-hydroxypicolinic acid (HPA), Anthranilic acid, Nicotinic acid, Salicylamide and mixtures thereof. The amount of applied matrix may vary although it is typically of the order such that the ratio of matrix to added sample is from 0.1:1 to 10:1, preferably from 0.5:1 to 5:1, most preferably from 1:1 to 2:1.

In another preferred embodiment the digestion is carried out on a carrier. In this embodiment the method preferably includes applying a proteolytic agent to a carrier prior to application of the material to the carrier such that following addition of the material the agent partially digests any polypeptides within the material. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used in the digestion however it is preferred that the proteolytic agent is a protease, preferably a protease selected from the group consisting of trypsin and endoprotease Glu C. In one preferred embodiment the digestion occurs in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed for. The digestion may be stopped in any way well known in the art. For example the digestion may be stopped by addition of a diluted acid either to the digestion in solution or to the on carrier digestion. An example of a suitable acid is TFA. A particularly preferred way of terminating the on carrier digestion is by applying a MALDI matrix over the material. Any suitable MALDI matrix may be used however the MALDI matrix is preferably selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxyacetophenone (THAP) and 3-hydroxypicolinic acid (HPA), Anthranilic acid, Nicotinic acid, Salicylamide or mixtures thereof.

The digested material is subjected to analysis by MALDI-ToF MS to determine digestion fragments for the material. Once the digestion fragments have been determined they are compared to the known digestion fragments (typically called the signature fragments) of the non variant polypeptides. Whilst this can be done manually by scanning the output of the MALDI-TOF MS and comparing it to digestion fragments of known non-variant polypeptides it is preferred that the comparison is carried out by computerised means. In a particularly preferred embodiment the output of the MALDI-ToF MS analysis is compared by computer means to a library of signature fragments for non variant polypeptides to determine the fragment containing the variation. Once the fragment has been determined it is generally straightforward to determine the nature of the variation.

In yet a further aspect the invention provides a method of diagnosing a condition in a subject including the steps of:
(a) obtaining a material to be analysed from a subject;
(b) analysing the material by MALDI-ToF MS to identify one or more polypeptides within the material,
(c) determining from the presence or absence of a polypeptide within the material whether the subject has the condition.

The condition to be diagnosed is either a condition that is characterised by the absence of a polypeptide that would be present in material obtained from a non-afflicted subject or a condition that is characterised by the presence in the material of a polypeptide characteristic of the condition, said polypeptide not being present in a sample of a non-afflicted subject. In a preferred embodiment the condition is a haemoglobinopathy. Haemoglobinopathies fall into overlapping groups: thalassemias (imbalance in globinchain production) and haemoglobin variants (structurally abnormal haemoglobins). Haemoglobinopathoies include: alpha-thalassemia (non-deletional, deletional, Hb H disease), beta-thalassemia, delta-thalassemia, gamma-thalassemia, hereditary persistence of fetal hemoglobin (HPFH), deltabeta-thalassemia, sickle cell disorder and other haemoglobin variant related disorders.

In principle the material obtained may be any bodily material or extract. Examples of materials that may be used include blood, CSF fluid, urine, saliva, seminal fluid or sweat or a combination thereof. It is preferred that the material is blood. The material is obtained from the subject using standard techniques well known in the art.

The material is then analysed by MALDI-ToF MS to determine polypeptides in the material. The analysing step preferably involves subjecting the material to be analysed to MALDI-ToF MS analysis on a carrier. The material on the carrier has preferably been subjected to a partial digestion.

The digestion may be carried out in solution prior to application to a carrier or may be carried out after the material has been applied to the carrier. Accordingly, the material may be digested either in solution or whilst on the carrier. In one preferred embodiment the digestion is carried out in solution by addition of a proteolytic agent to a solution containing the material. In this embodiment it is preferred that the digestion is carried out for from 1 to 24 hours, more preferably 4 to 24 hours. Any suitable proteolytic agent may be used in the digestion however it is preferred that the proteolytic agent is a protease, preferably a protease selected from the group consisting of trypsin and endoprotease Glu C. In one preferred embodiment the material is digested in the presence of a surfactant. The surfactant is preferably sodium 3-[(2-methyl-2-undecyl-1,3-dioxolan-4-yl)methoxy]-1-propane-sulfonate. The digestion may be stopped by any method well known in the art. Following the in solution digestion the digested material is then preferably applied to a carrier.

Following application of the material to the carrier at least a portion of the liquid component is removed. The liquid component may be removed in any suitable manner that does not destroy the integrity of polypeptides within the material. For example the liquid may be removed by subjecting the applied material to elevated temperature, reduced pressure or a combination thereof. The liquid may also be removed by passing a stream of gas (preferably air) over the surface o the applied material. In a particularly preferred embodiment the liquid is removed by allowing the applied material to sit at ambient temperature and pressure for a sufficient time for the liquid to be removed by evaporation.

The amount of liquid removed may vary. It is preferred that at least 50% of the liquid component is removed, more preferably at least 75% of the liquid component is removed, yet even more preferably at least 90% of the liquid component is removed. In another preferred embodiment removal of the liquid component continues until the material is substantially dry, more preferably removal continues until the material is dry. Following the liquid removal step a MALDI matrix is applied. Any suitable MALDI matrix may be used however it is preferred that the MALDI matrix is selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxy acetophenone (THAP) and 3-hydroxypicolinic acid (HPA), Anthranilic acid, Nicotinic acid, Salicylamide and mixtures thereof.

In another preferred embodiment the digestion is carried out on the carrier. In this embodiment the method preferably includes applying a proteolytic agent to a carrier prior to application of the material to the carrier such that following addition of the material the agent partially digests any polypeptides within the material. In this embodiment the digestion is preferably carried out for a period of from 10 to 3600 seconds, more preferably 30 to 600 seconds, more preferably from 60 to 300 seconds, most preferably for 180 seconds.

Any suitable proteolytic agent may be used in the digestion however it is preferred that the proteolytic agent is a protease, preferably a protease selected from the group consisting of trypsin and endoprotease Glu C. In one preferred embodiment the digestion in the presence of a surfactant. The surfactant is preferably sodium 3-[(2-methyl-2-undecyl-1,3-dioxolan-4-yl)methoxy]-1-propane-sulfonate.

The digestion is preferably allowed to continue until the digestion provides 100% sequence coverage of the polypeptide to be analysed for. The digestion may be stopped in any way well known in the art. For example the digestion may be stopped by addition of a diluted acid either to the digestion in solution or to the on carrier digestion. An example of a suitable acid is TFA. A particularly preferred way of terminating the on carrier digestion is by applying a MALDI matrix over the material. Any suitable MALDI matrix may be used however the MALDI matrix is preferably selected from the group consisting of sinapinic acid (SA), α-cyano-4-hydroxycinnamic acid (CHCA), 2,5-dihydroxybenzoic acid (2,5-DHB), 2-(4-hydroxyphenylazo)benzoic acid (HABA), succinic acid, 2,6-Dihydroxyacetophenone, Ferulic acid, caffeic acid, 2,4,6-trihydroxacetophenone (THAP) and 3-hydroxypicolinic acid (HPA), Anthranilic acid, Nicotinic acid, Salicylamide or mixtures thereof.

Once the sample has been prepared in the manner discussed above it is subjected to MALDI ToF MS analysis using standard operating conditions. The MALDI-TOF MS output is then analysed to determine from the digestion fragments the identity of one or more polypeptides within the material. The diagnosis of the condition is then based on the presence or absence of a polypeptide from the material. The output may be analysed using any of a number of techniques. At its most simplistic the output may be viewed manually to determine the digestion fragments and to determine if signature digestion fragments are present. It is preferred, however, that the output is compared using computer aided techniques with a database or library of known fragments. Any significant mass/charge signal representing a peptide, which is different from haemoglobin A, may constitute a Haemoglobin variant. If this variant is associated with a clinical significant characteristic it constitutes a haemoglobinopathy.

### BRIEF DESCRIPTION OF THE FIGURES

FIG. 1 shows MALDI-ToF mass spectra of haemoglobin α and β, chains, obtained from whole unpurified blood, diluted 1:100, showing the m/z values of double, single charged, dimers of the chains and adducts of single charged α and β chains in the linear mode.

FIG. 2 shows sequence coverage of α and β chain of Hb A standard at different time points course for a free solution digest.

FIG. 3 shows a MALDI-TOF mass spectrum obtained for the α and β chain tryptic fragments of the Hb A standard, from a 2 min free solution digest in the reflector mode.

FIG. 4 shows haemoglobin α chain (red) and β chain (green) sequence coverage in a time course experiment; A) Free solution digest; B) Free solution digest in presence of the surfactant; C) On carrier tryptic digest in the presence of sodium 3-[(2-methyl-2-undecyl-1,3-dioxolan-4-yl)methoxy]-1-propane sulfonate after incubation at 37° C. and D) On carrier tryptic digest in the presence of sodium 3-[(2-methyl-2-undecyl-1,3-dioxolan-4-yl)methoxy]-1-propane sulfonate after incubation at 100° C.

FIG. 5 shows MALDI-TOF mass spectra of tryptic peptides, in the reflector mode, obtained at time points 10 s, 30 s, 90 s and 180 s in an on carrier digest at 37° C., in presence of sodium 3-[(2-methyl-2-undecyl-1,3-dioxolan-4-yl)methoxy]-1-propane sulfonate, shown for the m/z range from 650-5650. The peaks were labelled automatically with a pre-programmed labelling file.

FIG. 6 shows MALDI-TOF mass spectra, in the linear mode, obtained at time points 10 s, 30 s, 90 s and 180 s in the on carrier digest at 37° C., in presence of sodium 3-[(2-methyl-2-undecyl-1,3-dioxolan-4-yl)methoxy]-1-propane sulfonate, shown for the m/z range from 5000-25000, to monitor depletion of α an β chain confirming active and rapid digest of the chains.

FIG. 7 shows the tryptic fragmentation pattern of the human Hb α chain, obtained by MALDI-TOF MS in the reflector mode, at different time points, in a time course on carrier tryptic digest experiment in the presence of sodium 3-[(2-methyl-2-undecyl-1,3-dioxolan-4-yl)methoxy]-1-propane sulfonate at 37° C. This figure corresponds to the time course depicted in FIG. 4, Panel D (Res.=Residues, % coverage=% sequence coverage).

FIG. 8 shows the tryptic fragmentation pattern of the Hb β chain, obtained by MALDI-TOF MS in the reflector mode, in a time course on carrier tryptic digest at 37° C. in the presence of sodium 3-[(2-methyl-2-undecyl-1,3-dioxolan-4-yl)methoxy]-1-propane sulfonate. This figure corresponds to the time course depicted in FIG. 4, Panel D. Res.=Residues,?=weak signal.

FIG. 9 shows MALDI-ToF mass spectra obtained from on carrier tryptic digest of A) 1:10, and B) 1:100 diluted unpurified whole blood in the presence of sodium 3-[(2-methyl-2-undecyl-1,3-dioxolan-4-yl)methoxy]-1-propane sulfonate 37° C.

FIG. 10 shows MALDI-ToF MS of proteolytic fragments derived from α and β chains from unpurified whole human blood using the reflector mode. The on carrier 3 min digest was carried out using endoproteinase Glu C in the presence of the novel surfactant at 37° C., shown in the m/z window 650-5650.

FIG. 11 shows MALDI-ToF mass spectra of βG1-2 (824.3936, pos 1-7), βG3 (1616.7608, position 8-22), βG2-3 (1745.9068, position 7-22) fragments derived from an on carrier Glu C digest of the β globin chain of Hb A from whole human blood showing cleavage of both Glu⁶ and Glu⁷. The digestion was performed in the presence of the novel surfactant sodium 3-[(2-methyl-2-undecyl-1,3-dioxolan-4-yl)methoxy]-1-propane sulfonate at 37° C. for 3 minutes.

FIG. 12 shows a MALDI-ToF mass spectrum of intact globin chains of whole unpurified Hb AE with a mass shift of 0.94 Da for the variant β_{E} showing that a separation of ββ_{E} was not achieved with the current specification of MALDI-TOF MS analyser.

FIG. 13 shows a MALDI-TOF mass spectrum in the reflector mode of an on carrier 3 min digest at 37° C. of whole unpurified (Hb E heterozygote) in the presence of the novel degradable surfactant, showing complete sequence coverage for all globin chains including β_{E}.

FIG. 14 shows a MALDI-ToF mass spectrum in the reflector mode, overlaid traces of two on carrier 3 min digests at 37° C. of whole unpurified Hb A (Green) and Hb E (Blue) in the presence of the novel degradable surfactant showing the appearance of the signature peptide β_{E}T3 VNVDEVGGK with a monoisotopic mass of 916.4715.

FIG. 15 shows a MALDI-ToF mass spectrum of intact globin chains of whole unpurified HbAC with a mass shift of 0.94 Da for the variant β_{C} showing that a separation of ββ_{C} was not achieved with the current specification of the MALDI-ToF MS analyser.

FIG. 16 shows overlaid mass spectrometric traces of two on carrier 3 min digests at 37° C. of whole unpurified Hb A (Green) and Hb AC (Blue) in the presence of the novel degradable surfactant showing the appearance of the signature peptide β_{C}T2-3, EKSAVTALWGK obtained by MALDI-ToF MS in the reflector mode.

FIG. 17 shows MALDI-TOF spectra of: A) Appearance of peak corresponding to the β_{C}T1-2 fragment (received m/z value 951.5748) in blood containing Hb AC; B) Absence of any peak before βT1 (received m/z value 952.4958).

FIG. 18 shows MALDI-TOF MS of intact globin chains of whole unpurified Hb S in the linear mode showing a split in the β chain. β and β_{S} were resolved with a grid voltage of 90% and a delay time of 350 ns in MALDI-ToF MS linear mode.

FIG. 19 shows [M+2H]⁺⁺/2 peaks resolved in MALDI-ToF MS linear mode for Hb AS.

FIG. 20 shows overlaid MS traces of normal (green) and Hb S from an on carrier 3 min tryptic digest at 37° C. in the presence of sodium 3-[(2-methyl-2-undecyl-1,3-dioxolan-4-yl)methoxy]-1-propane sulfonate showing the appearance of peak β_{S}T1 (received m/z value 922.2883) in blood containing Hb AS and the absence of any peak in the same m/z region in normal blood.

FIG. 21 shows overlaid MS traces of normal (green) and Hb S obtained in the MALDI MS reflector mode, of an on carrier 3 min tryptic digest at 37° C. in the presence of sodium 3-[(2-methyl-2-undecyl-1,3-dioxolan-4-yl)methoxy]-1-propane sulfonate showing the appearance of peak β_{S}T1-3 (received m/z value 3131.7227) in blood containing Hb S and the absence of any peak in the same m/z area in normal blood. The m/z value of 3124.4223 represents αT8-9 and the m/z value of 3161.4981 βT1-3. The homozygous state for the Hb S variant would be characterised by the absence of βT1 and βT1-3; and presence of only β_{S}T1 and β_{S}T1-3.

FIG. 22 shows MALDI-ToF MS of intact single charged globin chains of whole unpurified blood containing Hb α₂ ββ_{J-Bangkok} in the linear mode showing a split in the β chain. The β and β_{J-Bangkok} were resolved with a grid voltage of 90% and a delay time of 350 ns in the MALDI-ToF MS linear mode. Inset: Double charged intact globin chains with a split in the β chain.

FIG. 23 shows MALDI-TOF spectra of: A) Normal βT5 fragment, B) Normal βT5 and β_{J-Bangkok}T5 (received m/z value 2116.9597). Both MALDI MS reflector mode spectra were obtained from on carrier 3 min tryptic digests of Normal Hb A and Hb J Bangkok at 37° C. in the presence of sodium 3-[(2-methyl-2-undecyl-1,3-dioxolan-4-yl)methoxy]-1-propane sulfonate.

FIG. 24 shows MALDI-ToF MS of intact single charged globin chains of whole unpurified blood containing Hb αα_{Setif}β₂ in the linear mode showing a split in the α chain peak. The α and α_{Setif} chains were resolved using a grid voltage of 90% and a delay time of 350 ns in the MALDI-ToF MS linear mode. Inset: Double charged intact globin chains with a split in the α chain.

FIG. 25 shows overlaid MALDI MS reflector mode spectra of on carrier 3 min tryptic digests of Normal Hb A (green) and Hb Setif (blue) at 37° C. in the presence of sodium 3-[(2-methyl-2-undecyl-1,3-dioxolan-4-yl)methoxy]-1-propane sulfonate showing the appearance of α_{setif}T11, a signature peptide for identification of Hb Setif.

FIG. 26 shows overlaid MALDI MS reflector mode spectra of on carrier 3 min tryptic digests of Normal Hb A (green) and Hb Setif (blue) at 37° C. in the presence of sodium 3-[(2-methyl-2-undecyl-1,3-dioxolan-4-yl)methoxy]-1-propane sulfonate showing the appearance of α_{setif}T10-1 1, a signature peptide for the identification of Hb Setif.

FIG. 27 shows MALDI-ToF MS of intact single charged globin chains of whole unpurified blood containing Hb α₂ββ_{Ty Gard} in the linear mode showing a split in the β chain. The β and β_{Ty Gard} chains were resolved using a grid voltage of 90% and delay time of 350 ns in the MALDI-TOF MS linear mode.

FIG. 28 shows a typical Glu C fragmentation pattern and the appearance of the signature peptide following on carrier 3 min endoproteinase Glu C digest of Hb Ty Gard (α₂ββ_{Ty Gard}) at 37° C. in presence of sodium 3-[(2-methyl-2-undecyl-1,3-dioxolan-4-yl)methoxy]-1-propane sulfonate.

FIG. 29 shows the appearance of the signature peptide β_{TyGard}G9 (received m/z value 2711.4457) following on carrier 3 min endoproteinase Glu C digests of Normal Hb A (blue) and Hb Ty Gard (green) at 37° C. with sodium 3-[(2-methyl-2-undecyl-1,3-dioxolan-4-yl)methoxy]-1-propane sulfonate. This peak is absent in normal blood Glu C digest.

FIG. 30 shows MALDI-TOF MS of globin chains in the linear mode showing a split in the α chain peak. The α and α_{J-Toronto} chains were resolved having a mass difference of +44 Da.

FIG. 31 shows overlaid MS traces of a 3 min on carrier tryptic digestion of Hb J-Toronto (blue) and normal blood (green) obtained with the ionic surfactant sodium 3-[(2-methyl-2-undecyl-1,3-dioxolan-4-yl)methoxy]-1-propane sulfonate SF at 37° C. showing the resolved signature peptide α_{J-Toronto}G1.

FIG. 32 shows overlaid MS traces of a 3 min on carrier tryptic digestion of Hb J-Toronto (blue) and normal blood (green) obtained with the ionic surfactant sodium 3-[(2-methyl-2-undecyl-1,3-dioxolan-4-yl)methoxy]-1-propane sulfonate SF at 37° C. showing the resolved signature peptide α_{J-Toronto}G1-2.

FIG. 33 shows the appearance of the signature peptide α_{J-Toronto}G1-3 in an on carrier 3 min digest of a sample having a Hb J Toronto α chain.

FIG. 34 shows MALDI-ToF MS in the linear mode of globin chains showing a split in the β chain peak. The β and β_{J-Kaohsiung} chains were resolved having a mass difference of −27.07 Da.

FIG. 35 shows overlaid MS traces of a 3 min on carrier tryptic digestion of Hb J-Kaohsiung (blue) and normal blood (green) obtained with the ionic surfactant RapiGest™ SF at 37° C. showing the resolved signature peptides β_{J-Kaohsiung}T5 and β_{J-Kaohsiung}T5-6.

FIG. 36 shows MALDI-TOF MS in linear mode of globin chains showing a split in the β chain peak. The β and β_{Long Island} chains were resolved having a mass difference of 90.9 Da.

FIG. 37 shows overlaid MS traces of two 3 min on carrier endoproteinase Glu C digestions of Long Island (blue) and normal blood (green) obtained with the ionic surfactant sodium 3-[(2-methyl-2-undecyl-1,3-dioxolan-4-yl)methoxy]-1-propane sulfonate SF at 37° C. showing the resolved signature peptides β_{Long Island} G1-3.

FIG. 38 shows a MALDI-TOF mass spectrum of intact globin chains obtained from a sickle thalassaemia patient using the linear mode showing the α, α and γ chains respectively; peak areas are marked.

FIG. 39 shows a MALDI-TOF mass spectrum of intact globin chains obtained from a thalassaemia intermedia patient using the linear mode showing the α, the β, the δ and the γ chains respectively; peak bounds are marked.

FIG. 40 shows MALDI-TOF MS measurement of glycation in globin chains separately and in total.

FIG. 41 shows overlaid traces of MALDI-TOF mass spectra obtained from samples with high and normal glycation of globin chains showing increase peak height area for glycated α and β adducts.

FIG. 42 shows glycation of individual globin chains and in total, * indicates that the SA adduct area was included into the calculation of glycation proportion.

FIG. 43 shows a MALDI-TOF mass spectrum of an on carrier 3 min digest with Glu C in the presence of sodium 3-[(2-methyl-2-undecyl-1,3-dioxolan-4-yl)methoxy]-1-propane sulfonate at 37° C. of normal blood showing glycation and hydroxylated of βG8.

FIG. 44 shows a MALDI-TOF mass spectrum of an on carrier 3 min digest with Glu C in the presence of sodium 3-[(2-methyl-2-undecyl-1,3-dioxolan-4-yl)methoxy]-1-propane sulfonate at 37° C. of normal blood showing the absence of the normal βG8 peak.

FIG. 45 shows a MALDI-ToF mass spectrum of an on carrier 3 min digest with Glu C in presence of sodium 3-[(2-methyl-2-undecyl-1,3-dioxolan-4-yl)methoxy]-1-propane sulfonate at 37° C. of normal blood showing glycation and methylation of the fragment βG3-4.

FIG. 46 shows a MALDI-ToF mass spectrum of an on carrier 3 min digest with Glu C in the presence of sodium 3-[(2-methyl-2-undecyl-1,3-dioxolan-4-yl)methoxy]-1-propane sulfonate at 37° C. of blood sample with high glycation level showing absence of βG8.

FIG. 47 shows a MALDI-ToF mass spectrum of an on carrier 3 min digest with Glu C in the presence of sodium 3-[(2-methyl-2-undecyl-1,3-dioxolan-4-yl)methoxy]-1-propane sulfonate at 37° C. of blood sample with high glycation level showing glycation of βG8 (hydroxylated).

FIG. 48 shows a MALDI-ToF mass spectrum of an on carrier 3 min digest with Glu C in the presence of sodium 3-[(2-methyl-2-undecyl-1,3-dioxolan-4-yl)methoxy]-1-propane sulfonate at 37° C. of blood sample with high glycation level showing glycation of βG3-4 with increased signal intensity (methylated).

FIG. 49 shows MALDI-ToF mass spectra obtained from on carrier tryptic digests of blood diluted 1:100 with sodium 3-[(2-methyl-2-undecyl-1,3-dioxolan-4-yl)methoxy]-1-propane sulfonate at 37° C. showing appearance of βT1, βT2-3 and βT1-3 in A) With 1:20 dilution of trypsin, B) With 1:100 dilution of trypsin. Inset A Right. Disappearance of βT1-3 in 1:10 trypsin dilution (green) and presence of the peak in 1:100 trypsin dilution (blue).

FIG. 50 shows MALDI-TOF mass spectra obtained from on carrier tryptic digests of blood containing Hb S variant, diluted 1:100 with sodium 3-[(2-methyl-2-undecyl-1,3-dioxolan-4-yl)methoxy]-1-propane sulfonate at 37° C. showing appearance of β_{S}T1 and β_{S}T1-3; A) Appearance of the β_{S}T1 tryptic fragment with 1:20 trypsin dilution of stock trypsin solution; B) Appearance of the β_{S}T1-3 and βT1-3 tryptic fragments with 1:20 trypsin dilution of stock trypsin solution; C) A weak signal for the β_{S}T1 tryptic fragment with 1:10 trypsin dilution of stock trypsin solution; D) Disappearance of β_{S}T1-3 and βT1-3 in 1:10 trypsin dilution (blue) and presence of the peaks in 1:20 trypsin dilution (green).

FIG. 51 shows a typical tryptic fragmentation of α and β globin chains of normal adult Hb A obtained from 3 min on carrier digests of whole unpurified blood samples directly collected into ammonium bicarbonate buffer, 1:100 dilution, in presence of the novel surfactant.

FIG. 52 shows a MALDI-TOF mass spectrum obtained from blood with a variant in the linear mode using 1:100 diluted unpurified blood showing the intact α and β chain along with three additional peaks near the β chain.

FIG. 53 shows the appearance of the signature peptide β_{NewM1}T4 with an m/z value of 1191.6879 (expected m/z value 1191.6554) in a MALDI-ToF mass spectrum obtained from a 3 min on carrier tryptic digest in the presence of the novel surfactant at 37° C.

FIG. 54 shows MALDI-TOF mass spectra obtained from blood with a variant in the linear mode using 1:100 diluted unpurified blood showing the intact α chain and two poorly separated β chain peaks.

FIG. 55 shows MALDI-ToF mass spectra of a 3 min on carrier tryptic digest in the presence of the novel detergent of blood containing a new Hb variant showing the appearance of the signature peptide 3555.0594 (blue) and its absence in normal blood (green).

FIG. 56 shows overlaid MALDI-ToF mass spectra of 3 min on carrier tryptic digests in the presence of the novel detergent of blood containing a new Hb variant showing the appearance of the signature peptide 2272.9532 (green) and its absence in normal blood (blue).

FIG. 57 shows overlaid MALDI-ToF mass spectra of a 3 min on carrier tryptic digests in the presence of the novel detergent of blood containing a new Hb variant showing the appearance of the signature peptide 3328.5215 (blue) and its absence in normal blood (green).

FIG. 58 shows the signal to noise ratio of a number of digested globin chain peptide peaks obtained from normal blood sample diluted 1:00, 1:1000, 1:10000, 1:100000 and a 3 min on carrier digests with sodium 3-[(2-methyl-2-undecyl-1,3-dioxolan-4-yl)methoxy]-1-propane sulfonate at 37° C. showing the increase or decrease of signal to noise ratio at different dilutions.

FIG. 59 shows the obtained mass spectra from a 3 min on carrier tryptic digests of blood with dilutions 1:100, 1:1000, 1:10000 and 1:100000 with sodium 3-[(2-methyl-2-undecyl-1,3-dioxolan-4-yl)methoxy]-1-propane sulfonate at 37° C. using the MALDI-ToF MS reflector mode showing the gradual change of the signal intensities of the globin chain proteolytic fragments.

FIG. 60 shows overlaid MS traces of a 3 min on carrier tryptic digests of blood with dilutions 1:100 (green) and 1:100000 (blue) with sodium 3-[(2-methyl-2-undecyl-1,3-dioxolan-4-yl)methoxy]-1-propane sulfonate at 37° C. using the MALDI-TOF MS reflector mode showing the appearance of δT9-17 and βT1Acetylated fragments.

FIG. 61 shows a MALDI-ToF mass spectrum of a 3 min on carrier tryptic digests of blood with a dilution of 1:100000 with sodium 3-[(2-methyl-2-undecyl-1,3-dioxolan-4-yl)methoxy]-1-propane sulfonate at 37° C. showing the appearance of the γT1-8 fragment.

FIG. 62 shows a MALDI-TOF mass spectrum of a 3 min on carrier tryptic digest with the presence of the novel surfactant at 37° C. of unpurified blood containing normal adult Hbs showing the absence of any ζ fragments.

FIG. 63 shows a MALDI-TOF mass spectrum of a 3 min on carrier tryptic digest with the presence of the novel surfactant at 37° C. of unpurified blood from an α thalassaemia patient showing the presence of the ζT3 and the ζT3 fragments in a 1:10 dilution of blood.

FIG. 64 shows a MALDI-ToF mass spectrum of a 3 min on carrier tryptic digest with the presence of the novel surfactant at 37° C. of unpurified blood from an α thalassaemia patient showing the presence of the ζT3 and the ζT3 fragments in a 1:100 dilution of blood.

FIG. 65 shows a MALDI-TOF mass spectrum of a 3 min on carrier tryptic digest with the presence of the novel surfactant at 37° C. of unpurified blood from an α thalassaemia patient showing the presence of the ζT3 and the ζT3 fragments in a 1:1000 dilution of blood.

FIG. 66 shows overlaid MALDI-TOF mass spectra of a 3 min on carrier tryptic digests with the presence of the novel surfactant at 37° C. of unpurified blood from an α thalassaemia patient and a normal individual showing the absence of any ζT3 and ζT3 fragments in blood from an normal individual and the presence the ζT3 and the ζT3 fragments in blood from a thalassaemia patient.

FIG. 67 shows a Comparison of different spotting methods for intact Hb (1:100 diluted) A. Dried droplet method, with further dilution 1:1 (v/v) with 50% ACN water, scattered non homogenous large crystals; B. Reversed two-layer method, sample to matrix ratio 2:1, homogenous fine crystals; C. Dried droplet sample spot, dilution 1:1 (v/v) with 50% ACN water; and D. New spotting technique, blood dilution 1:10, non homogenous scattered large crystals.

FIG. 68 shows a MALDI-ToF mass spectrum of intact α and β chains obtained in the linear mode from 1:100 diluted blood sample colleted directly into the 50 mM ammonium bicarbonate, 2 mM CaCl₂, pH 8.3, buffer. The insert shows the m/z scan over the range 7,000 to 17,000.

FIG. 69 shows a MALDI-ToF mass spectrum showing poorly resolved intact α and β peaks, the sample was blood in 1:10 dilution, the matrix was SA.

FIG. 70 shows a MALDI-TOF mass spectra of the intact globin chains obtained from A, 1:100, B, 1:1,000, C, 1:10,000 dilution of blood, with the matrix SA.

### DETAILED DESCRIPTION OF THE INVENTION

The term “polypeptide” refers to a chain of amino acids, wherein adjacent amino acids are linked by peptide bonds. The amino acids may be naturally occurring amino acids or modified amino acids. Other terms such as “protein” or “peptide” are intended to be encompassed by the term “polypeptide”.

The methods of sample preparation and analysis of the present invention are applicable to a wide range of materials, however it is preferred that the materials include biological materials or are derived from biological materials. In a particularly preferred embodiment the material is a biological material.

Any suitable biological material may be used, however it is preferred that the biological material is selected from the group consisting of blood, cerebrospinal fluid, urine, saliva, seminal fluid, sweat and a combination thereof. These samples may be obtained using techniques well known in the art that need no further elaboration.

Once obtained the material is then typically diluted in a liquid, preferably water. The liquid preferably includes a buffer. A suitable buffer is ammonium bicarbonate and a suitable level of dilution is from 1:10 to 1:10000. This is found to provide a suitable level of material for further analysis by the techniques described herein.

As stated previously the sample preparation techniques and methods of analysis as described herein provide improvements in the performance of the analysis of the sample. They typically provide improved sensitivity and/or reproducibility of the analysis.

The sample preparation techniques and methods of analysis as described herein typically involve addition of a material to a carrier. The amount of material added may vary considerably depending on the final application but it is typically of the order of 0.1 to 10 μl, more preferably 0.5 to 5.0 μl, most preferably about 1 μl. Any carrier well known in the art may be used. Examples of suitable carriers include Stainless steel carrier plates, gold carrier plates, carrier plates with hydrophobic surfaces, carrier plates with surface indentations (used with gel membranes).

In a particularly preferred embodiment the carriers have a plurality of sample positions such that a plurality of samples may be added to the one carrier. This allows for rapid throughput analysis of a number of samples on a MALDI-ToF MS apparatus and therefore provides for an economic process to be carried out.

In order to perform the methods of the invention as described herein it is preferable to digest the material to be analysed so that any polypeptides in the material are cleaved into smaller peptides which are more amenable to MALDI-ToF MS analysis. For the methods of the present invention, the applicants have found that a partial digest is able to give rapid and consistently accurate analysis of the material to be analysed.

The optimal conditions under which the partial digest is carried out must be determined for each class of polypeptides to be analysed and will depend on the material to be analysed. The skilled addressee will readily understand how to perform test digests in order to determine suitable conditions. Details of such digests are described below in reference to haemoglobins and are illustrative of the method to be used on a use by use basis. Conditions that need to be considered include, but are not limited to, enzyme, buffer, temperature, polypeptide concentration and time of digestion.

The digestion may be carried out either in solution or on a carrier, or a combination thereof. The digestion typically involves contacting the material with a proteolytic material. There are a large number of proteolytic materials well known in the art and the appropriate proteolytic material will depend upon the polypeptides expected to be present in the material to be digested. In general a skilled worker will be able to select a suitable proteolytic material with little difficulty. The amount of proteolytic material to be used will depend on the speed of digestion required. Once again through routine experimentation this can be readily determined.

The digestion may be carried out prior to application to a carrier. In this embodiment the digestion is typically carried out in solution. The digestion typically is carried out for a period of time suitable to provide at least a partial digestion of the polypeptides. The length of time will vary based on the polypeptides present but is typically from 4 to 24 hours. The digestion is typically carried out at temperatures well known in the art, generally from 0 to 100° C., more preferably 10 to 75° C. The exact temperature chosen will depend on the nature of the proteolytic agent and its optimal temperature range.

The digestion may be stopped using any technique well known in the art. Exemplary of such a technique is the addition of an acid. The material is then applied to a carrier as described previously herein.

The material is preferably applied by a spotting technique which would be well known to a skilled worker in the field.

After the material has been applied to the carrier it is typical that a MALDI matrix is applied using standard techniques. Any suitable MALDI matrix may be used but it is preferably selected from the groups described previously.

The sample is then analysed using standard MALDI-TOF techniques to determine the digestive fragments of the material to be analysed.

In particular embodiments of the present invention the partial digestion may be performed in the presence of a surfactant. Preferably, the surfactant is sodium 3-[(2-methyl-2-undecyl-1,3-dioxolan-4-yl)methoxy]-1-propane-sulfonate.

The methods of the invention all involve analysis on the basis of characteristic fragments of the polypeptide of interest. These characteristic fragments are commonly known as “signature” fragments of the polypeptide. There are a number of advantages in analysing a partially digested material for the presence of signature fragments of this type. The principal advantage is that in general the presence or absence of a signature fragment is determinative of whether the polypeptide is present or absent. This is generally more reliable than analysing the undigested material as the resolution with non-fragmented samples is not as great. Accordingly the use of fragmentation analysis therefore provides significant advantages.

In general for a large number of polypeptides the signature digestion peptides are known from the art and libraries of such peptides are available. In circumstances where the signature peptides are not known it is relatively straightforward to determine their identity. This can either be done theoretically based on the expected cleavage points of the polypeptide (which will be determined by the proteolytic agent of interest) or by subjecting a standard sample of the polypeptide to digestion conditions followed by analysis to determine the signature peptides. In general therefore the signature peptides can be determined quite easily either by theoretical means or by routine experimentation. If experimentation is used it is preferable to use the same conditions in the determination of the signature fragments as will be used in the material analysis.

Of course, once the signature fragments of a number of polypeptides are known this information can be used in methods of determining the identity of polypeptides in a material. Accordingly, if a material is subjected to digestion and then analysed the output of the MALDI-TOF mass spectrometry will provide the digestion fragments of the polypeptides in the material. Comparison of this output to the signature peptides of the known polypeptides (preferably by computer) allows for the identification of many of the polypeptides in the material. This allows for the rapid analysis of a complex material containing a number of polypeptides.

A particularly preferred use of this methodology is to determine if a material contains a particular polypeptide of interest. This can be very useful as the presence of the polypeptide may be indicative of a medical condition. This involves comparison of the MALDI-TOF MS output with the signature peptide or peptides of the polypeptide of interest. If the signature peptide is present this is indicative of the presence of the polypeptide of interest.

The fragment analysis discussed above can also be used in polypeptide variant analysis. By comparing the fragmentation pattern of a polypeptide variant with the fragmentation pattern of non-variant polypeptides it is generally easy to determine the fragment containing the variation (as it will be new). Once this has been done analysis of the difference between the new fragment and the corresponding non-variant fragment can be used to determine the difference in the variant.

The analysis of polypeptide variants in this way of course provides the analyst with signature peptides of the polypeptide variant which can be used as further probes for the presence of that polypeptide variant in complex mixtures Finally, the ability to accurately analyse complex materials for the presence or absence of a polypeptide may be a useful diagnostic tool.

A number of medical conditions are characterised by a gene defect such that the gene is not expressed in the body. The direct physiological effect of this non-expression of the gene is the absence in the body of the polypeptides that would be expressed in the body of a person without the gene defect. Accordingly the ability to accurately analyse a biological sample for the absence of a polypeptide may be used diagnostically. This is done by analysing the output and determining if the signature fragment of the polypeptide is present. If the signature fragment is not present it can be concluded that the polypeptide was not present in the sample further indicating that the individual had the gene defect. Alternatively, quantitative data can be used to determine if the polypeptide is present but at a reduced level (in some instances the gene defect leads to reduced production of the polypeptide).

In a number of other conditions there is not the absence of gene expression, rather the gene produces a polypeptide variant that is indicative of the condition. In these instances it is more reliable to analyse the individual for the presence of the polypeptide variant which will not be present in a sample from a healthy patient. This is because in some clinical conditions the person produces a certain amount of the “normal” polypeptide as well as an amount of the polypeptide variant. Merely analysing the sample for the absence of the normal polypeptide would therefore not be conclusive.

The method may be applied to any condition (typically a genetic defect) which is manifested in the production of an abnormal polypeptide (or a polypeptide variant). In many instances the presence of variant polypeptides is well known in the art and the present invention provides an improved method for the rapid qualitative analysis of these variants. Once the presence of the variant has been confirmed (by the presence of the signature fragment of the variant) the diagnosis of the condition that the presence of that variant indicates can be made.

One family of conditions that can be diagnosed using this technique are haemoglobenopathies which are manifested in variations in the α and β globin chains. In this family in general the known haemoglobenopathies are well documented and the polypeptides characteristic of each haemoglobenopathy well characterised. As such analysis for the presence of the polypeptides can be used in the diagnosis of the particular haemoglobenopathy.

In order to demonstrate the applicability of the improved sample preparation techniques and analytical methods, haemoglobins have been analysed as an indicative class of polypeptides. While the Examples below concentrate on haemoglobins, the skilled addressee would readily understand the methodology explained and be able to apply the methods to other polypeptide systems. Thus the choice of haemoglobins is intended demonstrate the applicability of the methodology and in no way is intended to limit the scope of the present invention.

Haemoglobinopathies are a major public health problem causing significant ill health, disability and death among the world populations. It has been estimated that at least 7% of the world's population are carriers of haemoglobinopathies. With the completion of the human genome project attention has now turned to studies of genetic diseases and their contribution to ill health and suffering in the community. In multicultural societies such as Australia screening for haemoglobinopathies is of increasing public health importance. Methods for diagnosis and management of these conditions need to be simpler, more rapid and more cost effective.

In general the polypeptide analysis techniques that are currently available are typically slow and not suited to fast throughput analysis. This can be seen by reference to the diagnostic approaches employed to detect haemoglobinopathies. The utility of the different methods currently used depends on the intended purpose, the availability of resources and the type of available technology. Initial and follow-up tests in practice include full blood examination (FBE), solubility and sickling tests, HbA₂ and HbF quantification and determination of the ferritin level, currently being performed by electrophoresis, iso-electric focusing (IEF), high-performance liquid chromatography (HPLC) and DNA analysis. Detection of ζ-globin chains in the cord blood by enzyme-linked immunoassay (ELISA) for screening for α-thalassaemia has also been described.

Many of the heterozygous and homozygous states for haemoglobin (Hb) disorders do not change the red cell morphology. Clinically significant Hb variants are usually first observed by routine haematological procedures. A low Hb level, microcytosis, hypochromia, blood film findings (target cells, fragmented red blood cells (RBCs), nucleated RBCs) are useful for the detection of thalassaemia major, sickle cell disease and unstable Hbs and are still the main screening tool in many of the poorer third world countries. Red cell indices are used to screen for β- and α-thalassaemia carrier states. Low mean corpuscular volume (MCV) (<82 fL) and mean corpuscular haemoglobin (MCH) (<27 pg) are indicative of such cases when iron deficiency is excluded even though the blood Hb level may not be lower than normal. Haemolysis is indicated by raised reticulocyte count. Reticulocyte count is also useful to provide information on unstable Hb variants, HbH disease or sickle cell disease. A high Hb level and increased haematocrit (HCT) level indicate erythrocytosis, which along with appropriate clinical observations may suggest a Hb variant with high oxygen affinity. Although these methods have their merits in the clinical diagnostics, they provide mainly morphological descriptions, which give extremely limited information on Hb variants.

While the cell observation techniques described above can assist in the identification of the presence of a haemoglobinopathy, they cannot identify the particular haemoglobin variant present. Molecular studies are required to identify the haemoglobin variant, which in turn may allow specific treatment of a patient.

Electrophoresis is one of the oldest methods available for the screening for Hb variants, and typically is used to screen a small number of samples. It has been used for detection and quantification of Hb variants. Because different haemoglobins may migrate similarly under a given set of conditions, electrophoresis is usually performed at two different pH values and on two different supporting mediums. The usual choice is cellulose acetate electrophoresis at pH 8.4 and citrate agar electrophoresis at pH 6.0. Most laboratories use commercially available kits that allow both medium and pH (6.0 and 8.2) separations. Cellulose acetate electrophoresis enables provisional identification of Hb variants. However, many bands reflecting different Hb variants overlap (such as the band for HbS overlaps the band for HbD). The use of citrate agar electrophoresis (separates HbC from HbE) and knowledge of patients ethnic background (HbC is common in North Africa and HbE in South East Asia) improves interpretation of results. Quantification by densitometry is possible but not routine. Variants such as HbS can be quantified but this method is not accurate at a low percentage of abnormal Hb or for HbA₂ quantification. HbA₂ quantification by capillary zone electrophoresis (CE) and CE with isoelectric focusing (IEF, see below) has also been described. Separation of haemoglobins by electrophoresis is based on the relative charge of the αβ dimer and hence mutations that do not alter the charge may be “electrophoretically silent”. Electrophoresis is not a good detection method for fast moving variants such as HbH. Overall, electrophoresis methods are slow, insensitive and limited in versatility.

In aqueous solutions, a pH can be obtained by titration methods at which the net charge of a specific polypeptide or an amino acid is zero. This is the isoelectric point or pl. Isoelectric focusing is a polypeptide separation technique based on exploiting differences in pl values. Separation of Hb variants with similar charge has been achieved. It generates better resolution than electrophoresis. IEF has replaced the conventional electrophoretic methods used in many laboratories and has been used to identify a few Hb variants. Separation of polypeptides is achieved using a set of synthetic ampholytes with pl values that cover the range of the pls of the polypeptides to be separated, and a separation can be achieved with a pl difference of about 0.01 pH units on a support matrix. Even higher resolution is achieved with a pl difference of 0.001, if the ampholytes are bound to the matrix. The most commonly used IEF technique, not compatible with automation, is the application of multiple samples to a commercially prepared thin layer gel. IEF has the same limitations as electrophoresis methods. In common with electrophoresis methods, IEF methods provide no information on the molecular structure of the Hb.

Ionic and hydrophobic interactions of the sample with the supporting matrix are the basis of separation in ion exchange (IEX) and reversed-phase high-performance liquid chromatography (RP-HPLC) respectively. Hb can be isolated as an intact tetramer or the individual globin chains can be separated. HPLC has been used in the analysis of HbA₂ HbF, other Hb fractions in screening for thalassaemias, as well as the isolation, detection and characterisation of several other Hb variants. Cation exchange chromatography, automated pre-programmed cation exchange HPLC and reversed-phase HPLC are used in laboratories for presumptive identification of haemoglobinopathies and thalassaemias. For definitive diagnosis, it is necessary to however still necessary to perform a DNA analysis or amino acid sequencing. These methods are time consuming, and do not give detailed information on the molecular structure of the variant and cannot be readily employed for high throughput screening tasks.

The genetic approach for detection and confirmation of diagnosis is an alternative strategy to polypeptide-based techniques, most of which are presumptive, especially where a mutation causes production of an unstable Hb, The development of polymerase chain reaction (PCR) methodologies and nucleotide sequencing techniques allows Hb variant characterisation at the gene level. A variety of methodologies have been developed for the detection of point mutations or deletions of α and β globin chains using DNA derived from white blood cells, amniocytes or chorionic villous samples. Southern blot oligonucleotide hybridisation, endonuclease restriction enzyme cleavage analysis of PCR products, amplification refractory mutation system, Gap PCR of known mutations, denaturing gradient gel electrophoresis and direct sequencing for unknown mutations are commonly used techniques.

All of the above methods require as much as hours to days to complete analysis and obtain the final result and are technically complex procedures. Recently, a prenatal real time PCR diagnostic method using the LightCycler requiring less than three hours including DNA extraction from a foetal sample (when parental mutations are known) has been described. Although DNA analysis is a powerful tool for identifying mutations or deletions, known and unknown, it cannot identify post-translational modifications of the expressed haemoglobins, and can only retrospectively give information about the origin of such changes.

For the analysis of polypeptides such as Hb variants, complete sequence coverage in a particular mass/charge window rather than a complete digest is preferred, in order not to lose the fragments smaller than 500 Da. This may be achieved by controlled incomplete proteolytic digest yielding overlapping fragments. In the Examples below, deliberate and controlled incomplete tryptic digestion of Hb in blood was performed to obtain analysable fragments to achieve a high level of sequence coverage, as compared to complete proteolytic digestion. The smaller fragments or fragments which are known to precipitate or those which are difficult to detect, as for example αT12 αT13, βT10, βT12, were consequently captured, since they are joined to bigger, more soluble fragments. A 100% sequence coverage for both the α and the β chain was achieved with trypsin using this newly developed digest method. The results were reproducible even after 6 months of sample storage.

The digestion may be carried out in solution or in an on carrier mode. It has been found that an on carrier digest provides superior performance. An on carrier digest typically digest includes the following steps, 1 μl of sample is deposited on 2 μl air dried trypsin on a MALDI sample plate, incubated for catalysis, stopped, covered with matrix and analysed. A detailed time course investigation has revealed the identity of fragments produced and the overall sequence coverage obtained for a particular time point. This procedure has dramatically improved sequence coverage, decreased digest time and robustness of the digestion chemistry. The data show that the acid labile surfactant sodium 3-[(2-methyl-2-undecyl-1,3-dioxolan-4-yl)methoxy]-1-propanesulfonate considerably reduced the digestion time of Hb when used with unpurified whole EDTA-treated blood. In combination with an on carrier digest, and the use of this surfactant, a 100% sequence coverage could be obtained for both globin chains in the a digest time of 2-3 min. This sulfonate-based surfactant with a monoisotopic mass of 417.2281 Da is acid labile and degrades to two non-interfering by-products with masses of 238.0482 and 198.1978. Such degradability has been reported for other sulfonate surfactants used with MALDI-TOF MS. It has been recognized that buffer components and surfactants, impose MALDI-ToF MS compatibility problems in terms of ionisation suppression. The development of sodium 3-[(2-methyl-2-undecyl-1,3-dioxolan-4-yl)methoxy]-1-propane-sulfonate and other acid-labile surfactants, which can be actively degraded to non-interfering by-products, show a new adaptation and streamlining of chemicals and methods in proteomics.

Whilst investigating variation of sample concentration with dilutions 1:10 and 1:100 with sodium 3-[(2-methyl-2-undecyl-1,3-dioxolan-4-yl)methoxy]-1-propanesulfonate and a 3 min digest, since the ionic surfactant concentration was equal in both digests, it can be concluded that in the 1:100 dilution digest the incompleteness of the digest is achieved not due to a lack of surfactant, but rather due to its intrinsic properties. The surfactant may only be able to interact by disintegration of the proteins on the domain-domain or the tertiary structural level. This indicates that an additional robustness level can be achieved with invariance of the sequence coverage in relation to the blood concentration. The computational analysis of the spectra of other blood proteins within the 25 highest MOWSE scores show that for each of the two dilution levels different proteins were identified by the Protein Prospector software. Further experiments and data analysis is essential to identify blood signature peptides other than those described from Hb for a particular dilution.

Besides the use of trypsin for on carrier 3 min digest in presence of sodium 3-[(2-methyl-2-undecyl-1,3-dioxolan-4-yl)methoxy]-1-propanesulfonate at 37° C. endoproteinase Glu C was investigated with success. The fragments produced by an on carrier Glu C digest with the particular conditions used in this invention enhance the overall peptide mapping capability

High quality mass spectra were obtained using automated data acquisition with set criteria. Rapid data acquisition with high resolution and signal to noise ratio was achieved without failure resulting in a high number of proteolytic peptides being identified within 10 ppm mass window. To test the robustness of the proteolytic method, various trypsin to sample ratios were investigated for on carrier 3 min digestion with sodium 3-[(2-methyl-2-undecyl-1,3-dioxolan-4-yl)methoxy]-1-propanesulfonate. The results show that varying the trypsin concentration from 5.45 pM/μl to 0.05 pM/μl did not alter the proteolytic fragmentation patterns adding to the robustness of the method.

Appearances of tryptic autolytic fragments have been reported in the literature. In this invention, a few autocatalytic fragments of trypsin were seen but to a much lesser extent than reported previously. This was most likely because of the short digest time due to the use of sodium 3-[(2-methyl-2-undecyl-1,3-dioxolan-4-yl)methoxy]-1-propanesulfonate and the high abundance of Hb in blood. The appearance of the autolytic fragments was sample to trypsin ratio dependent whereby decreased trypsin concentration or increased sample amount decreased the appearance of tryptic autolytic fragments. The autolytic fragments are thus not suitable candidates for internal calibration in the newly established method.

The methods developed have been used to identify a number of Hb variants. A total of 11 different α and β chain variants were identified by this method (Tables 1 and 2).

TABLE 1
List of the α chain variants identified with the MALDI-ToF MS
and amino acid sequence of the tryptic fragments with substitutions.

| | | | |
|---|---|---|---|
| T1 | T2-11 | T11 | T12-14 |

| | | |
|---|---|---|
| Tryptic fragments | 1-7 | 93-99 |
| Hb variants | 1 | 1 |
| Globin chain sequence | VLSPADK | VDPVNFK |
| Sequence with | VLSPNDK | VYPVNFK |
| substitutions | | |

TABLE 2
List of the β chain variants identified with the MALDI-ToF MS and
amino acid sequence of the tryptic fragments with substitutions.

| | | | | | | | |
|---|---|---|---|---|---|---|---|
| T1 | T2 | T3 | T4 | T5 | T6-12 | T13 | T14-15 |

| | | | | | |
|---|---|---|---|---|---|
| Tryptic | 1-8 | 18-30 | 31-40 | 41-59 | 121-132 |
| fragments | | | | | |
| Hb variants | 3 | 1 | 1 | 3 | 1 |
| Globin chain | VHLTPE | VNVDEV | LLVVY | FFESFGDLST | EFTPPVQAA |
| sequence | EK | GGEALGR | PWTQR | PDAVMGNPK | YQK |
| Sequence | MVPLTP | VNVDEV | LLVVY | FFESFGDLST | EFTGPVQA |
| with | (K/V)EK | GGLALGR | PCTQR | PDALMDNPT | AYQK |
| substitutions | | | | | |

Overall, the results demonstrate the general applicability of the 3 min on carrier proteolytic digest in the presence of the novel surfactant sodium 3-[(2-methyl-2-undecyl-1,3-dioxolan-4-yl)methoxy]-1-propanesulfonate at 37° C. for the identification of Hb variants.

### MALDI-ToF MS Analysis of Intact Globin Chains

The consistency in mass accuracy achieved by MALDI-TOF MS was remarkable (±5 Da) for intact globin chain analysis of Hb variants using this methodology. The intact globin chains, the matrix adducts and glycated globin chain adducts were well separated. The present applicants found that a better peak separation for globin chains, whilst resolving a variant heterozygous state, was achieved with a grid voltage of 90% and a delay time of 350 ns. It was evident from the spectra obtained in the linear mode for the variants observed that, although a high mass accuracy was achieved, a mass shift of <5 dalton cannot be identified with confidence, with the current specification of the MALDI-TOF MS instrument that was available. As such, whilst a protein identification can be established with a 10 ppm mass accuracy of any of its peptides greater than 11 amino acid residues in size from a MALDI-TOF MS spectrum in the reflector mode, the unambiguous proof of the absence of protein mutations requires both the determination of the mass of the protein in the linear mode and the complete coverage of the sequence obtained from proteolytic peptide mapping.

It was also observed that the peak area and relative intensity for the a and βchain was consistent for an individual sample and was highly reproducible for the same sample. The peak intensity and peak area for the α chain was persistently higher than for the β chain with a consistent α:β ratio in agreement with reports in the literature.

### Quantitative Aspects of MALDI-ToF Linear Mode MS in Relation to Intact Globin Chain Analysis.

It was demonstrated that MALDI-TOF MS measurements to quantify Hb chain levels were possible by measuring the peak areas, although low abundance haemoglobins (<1%) cannot be quantified with current instrument settings. The quantitative utilities of MALDI-TOF MS have been reported in the literature. Analysis of the heterozygous state of the Hb S and sickle thalassaemia to quantify respective haemoglobin levels reflected similarity of results obtained with HPLC.

### Analysis of Glycated Globin Chains

Glycated haemoglobin chains were also investigated to evaluate the quantitative aspects even further. It was observed that both the α and the β chain were glycated. It was also demonstrated in the experiments that glycation level was higher in the β chain than in the α chain. It was noted that there was a clear elevation of the glycated haemoglobin percentage in diabetic patient samples in agreement with reports in the literature. The MALDI-ToF MS measurements of glycated α and β chains resulted in a slightly higher percentage than reports obtained by a HPLC method, which only measures HB A1_{C} (β chain only), whilst MALDI-TOF MS measurement was calculated using the whole pool of glycated globin chains. The MALDI-ToF MS measurements of only the glycated β chain were closer to the results obtained by an HPLC method, although it was observed that the MALDI-ToF MS measurements of glycated β chain were always lower than that of HPLC. Similar finding have been reported by Lapolla et al. In contrast to Lapolla, no globin chain preparation was employed and SA adducts were separated which was not reported by these authors. Furthermore, in contrast to Lapolla, the MALDI-ToF mass spectra obtained resolved the α, β and the glycated globin chains with a mass accuracy of 1.5 Da. Repeated testing resulted in the remarkable reproducibility of the area measurement (SD 0.01%). It was interesting to see that the sample with a HPLC A1_{C} of 8.8% gave a higher MALDI-ToF MS measurement (14.71%) than the sample with a HPLC A1_{C} of 10.0%. It was noteworthy that the glycated p globin chain MALDI-ToF MS measurement for both the samples were near to the results obtained by HPLC, where by the sample with HPLC reported percentage of 8.8% had a high a chain glycation.

Whilst investigating the two identified p globin glycated peptides derived by an on carrier 3 min endoproteinase Glu C digest in the presence of sodium 3-[(2-methyl-2-undecyl-1,3-dioxolan-4-yl)-methoxy]-1-propanesulfonate, it was observed that only the proteolytic derived glycated fragment βG8 Gluc-Hydr show an increased ratio for the glycated sample for peak areas, peak heights and relative intensities when compared with the respective values from the adjacent peak βG4-5. The proteolytic peptide fragment βG3-4 Gluc-Meth did not show any difference between the normal and the sample with high glycation level. The glycation of peptides may affect tryptic fragmentation pattern by blocking particular cleavage sites and the mass spectra may contain new glycated peaks.

If one uses the direct analysis approach, whereby the sample is merely diluted, then the relative proportion of a particular Hb chain (γ, δ, ζ) in relation to the α chain and β chain remains constant. This is a definite advantage if quantitation is the aim. In attempting low abundance detection of the ζ chain peptide fragments, the aim was not to achieve a particular sequence coverage, or detect ζ chain variants. The aim was to find the detection conditions, where pathological levels of the ζ chain could be detected in relation to normal globin chains levels.

In determining detectability of proteolytic peptides from digests of low abundance proteins, it was demonstrated that tryptic fragments of both the α and the β chain can be detected from digests performed with a 1:100000 dilution of whole human blood without purification. The low abundances of δ and γchains make the peptides derived from enzymatic digests of these chains difficult to detect, yet in this study, the detectability of the ζ chain in blood samples from patients with α thalassaemia was investigated. Huisman et al. reported elevation of ζ chain level in α globin gene mutations. The presence of embryonic ζ chain in adults has been used as a marker of the presence of a thalassaemia, and an ELISA method has been reported to detect the embryonic ζ chain in α thalassaemic individuals. Three different dilutions of blood samples, three from patients having a --/αα (-α^{3.7}/-α^{3.7}, -α^{3.7}/--^{SEA}) gene deletion and one normal haemoglobin from blood of a healthy individual, 1:10, 1:100 and 1:1000 with ammonium bicarbonate buffer, were investigated with successful identification of the ζT3 and the ζT5 in all three samples in all dilutions when 50 spectra were accumulated. The mass accuracy of the identified ζ chain fragments was low which is expected because of the extremely low abundance of the ζ chain fragment ions. The presence of the ζT3 and the ζT5 in all three dilutions and the absence of any ζ tryptic fragments in the normal blood sample spectra established MALDI-ToF MS as a potential screening tool for two gene deletion α thalassaemia, where an elevation of ζ chain levels is reported.

Thus, as discussed above, the present invention provides improved methods for polypeptide analysis. Particular applications of these new methods include the analysis of polypeptide variants. The present invention therefore provides for the use of these methods in the analysis of polypeptide variants. Also provided by the present invention are methods of diagnosis incorporating the methods of the present invention.

Various embodiments of the present invention will now be discussed by reference to the following non-limiting examples. While these examples focus on haemoglobin analysis, it is to be understood that the use of haemoglobin is illustrative and not to be taken as limiting the invention in any way. Haemoglobin has been chosen as it represents a class of polypeptides which demonstrates many well characterised variants. Furthermore, the usefulness of techniques of the present invention can be demonstrated to clearly discriminate between these many variants. The skilled addressee will recognise the applicability of these techniques to other polypeptides.

### EXAMPLES

Throughout the specification and examples the following abbreviations are used.

### Abbreviations

ACN Acetonitrile

CHCA α-Cyano-4-hydroxycinnamic acid

CID Collision-induced dissociation

DE Delayed extraction

EDTA Ethylenediamine-N,N,N′,N′-tetraacetic acid

ELISA Enzyme-linked immunoassay

ESI Electrospray lonisation

Da Dalton

DHB Dihydroxybenzoic acid

DNA Deoxyribonucleic acid

Hb Haemoglobin

HPLC High-performance liquid chromatography

IEF Iso-electric focusing

LC Liquid chromatography

MALDI Matrix-assisted laser desorption/ionisation

min Minutes

MOWSE Molecular weight search

MS Mass spectrometry

m/z Mass-to-charge ratio

PSD Post source decay

SA Sinapinic acid

s Seconds

ToF Time-of-flight

TFA Trifluoroacetic acid

αCHCA α-Cyano-4-hydroxycinnamic acid

ppm Parts per million

### Apparatus

Whole human blood samples, Hb standard and all the proteolytic digest products were analysed with a Voyager DE-STR MALDI-TOF mass spectrometer from Applied Biosystems, Framingham, Mass., U.S.A. The instrument was chosen because it has the highest mass accuracy amongst currently available MALDI-TOF instruments. The system uses a 337 nm nitrogen laser using 3-nanosecond duration pulses with a maximum firing rate of 20 Hz. The mass analyser is equipped with the Voyager DE-STR Biospectrometry Workstation software. All samples were spotted on 100 well stainless steel plates. A Perkin Elmer Cetus DNA thermal cycler from Narwal, U.S.A. was used for sample incubation and as a hot plate. A hot air oven from Watson Victor Ltd, Australia and water baths from Grant Instruments (Cambridge) Ltd, Cambridge, U.K. were used for incubation of sample plate and samples respectively: The balance used for measuring reagents was from Eppendorf, Netherler Hinz GmbH, Germany (Mettler Toledo AG245), the centrifuge (Biofuge B) from Heraeus Christ, Germany and the pH meter (pH 20) from ATI Orion Research, U.S.A. To measure the glycated Hb percentages high performance liquid chromatography with the TOSOH Glycohaemoglobin analyser HLC-723 GHbV A1c 2.2, Japan was used.

### Chemicals and Reagents

Human Hb A standard [9008-02-0] as well as proteins and peptides used as calibration standards, ie, angiotensin 1, ACTH (1-17), ACTH (18-39), ACTH (7-38), bovine insulin, thioredoxin (E. coli), equine apomyoglobin, were obtained from Sigma Chem. Co. (St Louis, Mo., U.S.A) to be used as calibration standards. The calibration standards were dissolved in ACN:H₂O (50:50) (dilution) (v:v), 0.1% TFA. Proteolytic enzymes, bovine trypsin (10000 BAEE units/mg) [9002-07-7], endoproteinase Glu C [66676-43-5] and endoproteinase Asp N [9001-92-7] were obtained from Sigma Chem. Co. (St Louis, Mo., U.S.A). Ammonium bicarbonate and calcium chloride were obtained from BDH Chemicals (Kilsyth, Australia). Matrices α-Cyano-4-hydroxycinnamic acid (CHCA) [28166-41-8], 3,5-dimethoxycinnamic acid (Sinapinic acid, SA) [530-59-6] were obtained from Agilent (Forest Hill, Victoria, Australia) and 2,5-Dihydroxybenzoic acid [490-79-9] from Sigma Chem. Co. (St Louis, Mo., U.S.A). RapiGesT™ SF [308818-13-5], the ionic surfactant sodium 3-[(2-methyl-2-undecyl-1,3-dioxolan-4yl)methoxy]-1-propanesulfonate, was obtained from Waters (Rydalmere, NSW, Australia). Acetonitrile [75-05-8] (HPLC grade) and methanol [67-56-1] (HPLC grade) were obtained from Biolab Scientific Pty Ltd (Sydney, Australia). Trifluoroacetic acid (TFA) was obtained from Auspep Pty Ltd (Melbourne, Victoria, Australia). Water used for the study was distilled and deionised in a Milli-Q water purification system (Millipore, Bedford, Mass., U.S.A.).

### DNA Sequencing and HPLC

HPLC and DNA sequencing was performed using standard protocols at the Monash Medical Centre. The results were used to select a variety of Hb variants to build up a database of identifiable Hb aberrations with mass spectrometry.

### Computational Methods

Accessible surface area for the amino acids from the globin chains of human Hb was calculated from the 1A3N file identifier taken from the Brookhaven Protein Data Bank (PDB) available at http://www.rcsb.org/pdb/ that utilizes the SCRIβT1 program available at http://www.bork.embl-heidelberg.de/ASC/asc2.html. The monoisotopic mass differences were calculated using the following atomic masses of the most abundant isotope of the elements, C=12.0000000, H=1.0078250, N=14.0030740, O=15.9949146, P=30.9737634 and the average masses were calculated using the following atomic weights of the elements C=12.011, H=1.00794, N=14.00674, O=15.9994, P=30.97376, S=32.066.

### Nomenclature

The numbering system of the sequence position used to describe the peptide fragments derived from the digests is the common protein-based description. In this system the amino acid after the initiator methionine is number 1 and the tryptic, endoproteinase Glu C and endoproteinase Asp N fragments are numbered according their occurrence in the amino acid sequence starting from the N-terminus.

### MALDI-ToF Mass Spectrometry and Data Analysis

Different instrument settings were systematically investigated to for high quality data acquisition.

### Linear Mode

Spectra were obtained with delayed extraction using a delay time of 250-350 ns, a grid voltage of 85% to 90%, with positive polarity. The mass range was 5000-100000 Dalton with a lower mass gate set at 5000 Da for mass data acquisition. Each spectrum was obtained with 500 laser shots by accumulating 5 spectra each obtained by 100 laser shots. Otherwise, automated spectra acquisition was used to collect 10 spectra, each spectrum obtained by 100 laser shots, using defined selection criterion for each spectra. Each spectrum was accumulated when it passed the selection criteria of minimum resolution of 200, 300 or 500, a minimum signal intensity of 10000, a maximum signal intensity of 64,000. The laser intensity was varied from 2500 to 3000. Central bias was used for automated data acquisition. 10 consecutive spectra without any selection criterion were accumulated using automated spectra acquisition for sample spectra failing to pass selection criteria. Manual acquisition was used for non-homogenous sample spots.

### Reflector Mode

Spectra were obtained with delayed extraction using a delay time of 250 ns with positive polarity. The grid voltage was set at 85%. The mass acquisition mass range was 650-10000 Dalton where the low mass gate was set at 500 Da. Again, each spectrum was obtained with 100 laser shots and 5 consecutive spectra were accumulated. Automated spectra acquisition was used to collect either 10 or 50 spectra, each spectrum obtained by 100 laser shots, using defined selection criterion for each spectrum. Each spectrum was accumulated when it passed the selection criteria for selected peptides of a minimum resolution of 8000-10000, a minimum signal intensity of 1000 and a maximum signal intensity of 64000 for the base peak. The laser intensity was fixed to 2400 and central bias was used for automated data acquisition.

### Data Analysis

The resulting spectra were processed with the Data Explorer Software, Version 4.0.0.0, for baseline correction, noise filtering/smoothing and de-isotoping with the generic formula C₆H₅NO. Spectra were analysed using the ProteinProspector software ver. 3.2.1 using various settings to test automated identification of high and low abundance haemoglobins. For further analysis the 50 most intense peaks above a base peak intensity of 0%, 1% and 2% were considered. In this procedure the identity search mode was utilized were the IntelliCal routine utilises two filters for the obtained peaks list allowing for a maximum of five missed cleavage sites. Other setting for the procedures were requirement of a minimum of two peptides for a protein identification (considering the possibility of an acetylated N-terminus), allowing a protein molecular mass range from 1000-100000 Da, the pre-processing filter set to a mass accuracy of 150 ppm and the post-processing filter were set to a final mass accuracy of 10 ppm. For the automated detection of Hb ζ chain, the pre-processing filter was set to a mass accuracy of 400 ppm and the post-processing filter was set to a final mass accuracy of 250 ppm, the mass range to 5000-16500, and the pl range to 6.5-9. The peak filter was used to exclude the masses (m/z) below 650. This filtering was necessary as in Hb or blood digest, the heme group signal was overpowering the spectra most likely acting as an energy sink. The databank used for the identification of the Hb peptides was SwissProt mar03 and NCBInr.Mar03. Another search within the genepept 11299 databank was also conducted with the same settings. To automatically identify and label proteolytic fragments, a labelling file was created using the ‘create macro’ function of the Data Explorer Software, Version 4.0.0.0, containing the theoretical masses of [symbol] , [symbol] , [symbol] , γ, ζ globins, tryptic, Glu C and Asp N fragments up to five missed cleavages, their post-translational modifications and some possible artefacts masses. Peak area, ion count, peak resolution, peak height and peak relative intensity was calculated using the Data Explorer Software, Version 4.0.0.0.

### Sample Collection Procedure

Whole human blood samples collected in the haematology and the clinical genetics laboratories of Monash Medical Centre for electrophoresis, HPLC and DNA study were used. The samples were collected in EDTA (1.5±0.25 mg/ml of blood) containing vacutainers. These samples were then further subjected to mass spectrometric analysis. 5 μl of each of the blood samples was collected in eppendorf tubes from these laboratories and transferred, in iced containers. To investigate the stability of diluted whole blood in respect to MALDI-TOF MS analysis, blood samples diluted 1:100 in 50 mM ammonium bicarbonate buffer, 2 mM CaCl₂, pH 8.3, stored in cold room and analysed at different time points. In order to trial a comparatively simple sample collection procedure with volumes smaller than 1 μl, 0.5 μl of blood was collected from two individuals using a pipette and blood was directly added to 50 mM ammonium bicarbonate buffer, 2 mM CaCl₂, pH 8.3. The lysed blood was stored in a cold room for further analysis at different time points.

### Sample S

All samples were stored in a cold room at +4° C.

### Sample Preparation

The only pre-MS sample preparation was dilution of blood. 1 μl whole human blood in EDTA, diluted 1:10, 1:100, 1:1000 and 1:10000 with buffer (50 mM ammonium bicarbonate buffer, 2 mM CaCl₂, pH 8.3) or with deionised water for linear mode MALDI-TOF MS analysis of intact globin chains, adducts and post-translational modifications. To investigate the detectability and optimise the sample concentration in the reflector mode, samples were diluted 1:100, 1:500, 1:1000, 1:5000, 1:10000, 1:50000 and 1:100000 in ammonium bicarbonate buffer and proteolytic digestion was performed for each dilution in presence of a novel degradable surfactant.

### Example 1

### Investigation of Different Sample Preparation Methods

Optimal sample preparation is a prerequisite for successful MALDI-ToF mass spectrometric analysis of peptide and protein samples. Variables associated with a good sample preparation to achieve high quality mass spectrometric data have been widely investigated for biological samples. In this invention, the sample preparation typically involves a dilution of whole human blood, which is the first step of the analysis of intact globin chains of haemoglobin [or of the proteolytic digestion products of the globin chains] and was systematically investigated. Anticoagulant EDTA-treated whole blood was used because this sample collection protocol is standard in clinical laboratories. Blood was investigated without any purification, and as such, no electrophoretic or chromatographic sample purification procedure was employed.

The amount of blood used in this investigation was 1 μl per sample. The samples were diluted and kept at 4° C. and subjected to experimental procedures at different time points.

Choice of matrices, sample matrix preparations and spotting methods are of utmost importance to achieve high resolution and high accuracy in mass measurements. Different sample spotting methods were investigated to achieve the desired resolution followed by further systematic investigations to improve and optimise each step of the Hb or Hb variant identification as described in the following sections.

Whole human blood in EDTA, diluted 1:10, 1:100, 1:1000 and 1:10000 with buffer (50 mM ammonium bicarbonate buffer, 2 mM CaCl₂, pH 8.3) or with deionised water was spotted on the sample plate using different sample spotting methods described in the literature namely the two layer method, the sandwich method and the dried droplet method.

The samples were spotted with the two-layer technique by successive spotting 2 μl or 1 μl of either the matrix sinapinic acid (SA) or otherwise α-cyano-4-hydroxycinnamic acid (CHCA) and 1 μl of sample to have a matrix to sample ratio or 2:1 and 1:1 respectively.

For the dried droplet sample spotting method, 2 μl of sample and 2 μl of either the matrix SA or alternatively α-CHCA was mixed together, the sample-matrix mixture was further diluted 1:1, 1:5 and 1:10 with 50% ACN followed by deposition of 2 μl of this premixed sample matrix mixture on the sample plate for MS analysis.

For the sandwich sample spotting method, 1 μl of either the matrix SA or otherwise α-CHCA was spotted, air dried, followed by 2 μl of sample which was also air dried, which then followed by another 1 μl of either matrix on top of it.

A new sample spotting method was developed using a reversed-two-layer sample-spotting technique, whereby 1 μl whole human blood, diluted 1:10, 1:100, 1:1000 and 1:10000 with ammonium bicarbonate buffer, or with deionised water, was spotted on the sample plate, allowed to air dry, followed by addition of either 0.5 μl or 1 μl of SA. The in-solution tryptic digests were spotted using the reversed two layer method as well. The same reversed layer method was applied to analyse the on carrier digests in contrast to the commonly used method whereby matrix is directly added to the liquid analyte.

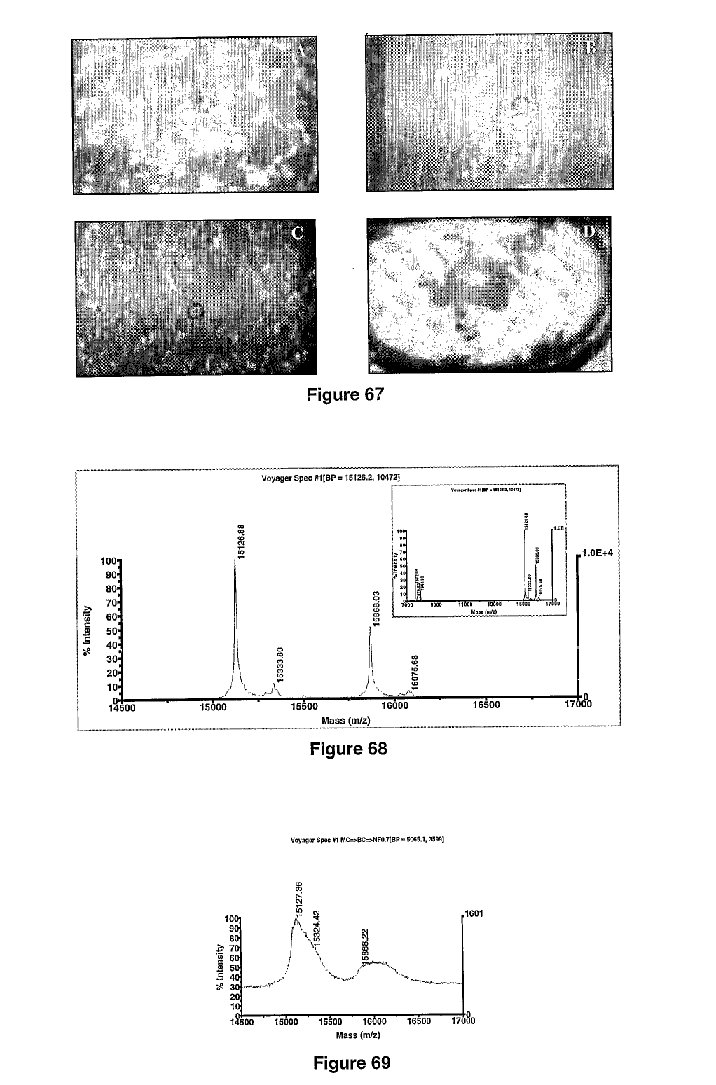

Variation in the sample-matrix crystallisation patterns with the different sampling methods was observed using diluted blood as the sample and SA as the matrix, as shown in FIG. 67A-C. In this new reversed two-layer sample spotting method, opposed to the two layer method and the sandwich method, as shown in FIG. 67B, the sample matrix co-crystallisation was apparently homogenous. The homogeneity varied from sample spot to sample spot on the MALDI sample plate for the dried droplet method, whereby homogeneity increased with less dilution, as shown in FIGS. 67A and 67C. For the premix sample spotting method, large scattered crystals were formed in diluted conditions, whereby, increased concentration of matrix in the sample mixture, 1:1 (v:v), with 50% ACN water, resulted in a dense sample spot with increased homogeneity. In the newly developed reversed two-layer method [the subject matter of this invention] the sample was spotted first followed by 0.5 μl of SA, which resulted in a thin layer of homogenous crystals on the sample spot. The variation of the sample concentration changed the spot homogeneity, whereby a higher sample concentration, as in a 1:10 dilution of blood in EDTA with ammonium bicarbonate buffer, decreased spot to spot reproducibility with a poor resolution of the Hb analyte and heterogeneously thick crystals on the sample spot, as shown in FIG. 67D. This scenario was reversed with a decreased sample concentration, a 1:100 and 1:1000 dilution of blood in EDTA with ammonium bicarbonate buffer, which resulted in a thin homogenous crystal layer on the sample spot.

Whilst comparing (Table 3) different sample spotting methods, the dried droplet, the two-layer method the sandwich method and the new technique in this application, the new spotting technique gave the best results. The methods were compared in respect to signal to noise ratio, resolution, ion abundance and time taken to accumulate a defined number of spectra (5, 10 and 50) with set selection criteria. These significant modifications that have lead to the new sample spotting method have not previously been discovered. Although the specific case of Hb's have represented the model system to establish this new technique, it should be noted that the same methodology should be applicable to other proteins and their derived tryptic (enzymatic) fragments when they are analysed in the linear and reflector mode of MALDI-ToF mass spectrometric analysis.

TABLE 3
Comparison of the different sample spotting methods.

| | | | | Time taken to |
|---|---|---|---|---|
| | | | | accumulate 10 |
| | | | | spectra with set |
| | | | | criteria |
| Spotting method | Signal to noise ratio | Ion count | Resolution | Time in s (SD) |
| Two layer | 14.7(2.4) | 705.5(123.9) | X | X |
| Sandwich | 1700.3(1321.2) | 7060.8(10372.9) | 202.1(116.4) | 6000+ |
| Dried Droplet | 5020.7(1524.3) | 25363.6(7775.2) | 495.8(146.1) | 258.7(84.6) |
| New Spotting | 5316.5(501.1) | 26700(3019.4) | 537.1(57.3) | 94.2(28.4) |
| Technique | | | | |

Sample: Whole EDTA treated blood, 1:100 dilutions.
Number of spectra: 10, each spectrum is an accumulation of 5 or 10 spectra.

The results demonstrate that the new spotting method described herein, whereby the diluted blood sample was spotted first, air dried, and then overlaid with the matrix (preferably sinapinic acid (SA)) using a sample matrix ratio of 2:1, substantially higher ion counts, higher resolution and excellent signal to noise ratios in the mass spectra were obtained both in the reflector and in the linear mode. This method gave a thin homogenous layer of sample matrix co-crystallisation, resulting in high spot-to-spot reproducibility with no obvious ‘hot’ [i.e. sample concentration non-homogeneity] spots. The fine microcrystalline coverage of the sample spot was best suited for an automated data acquisition whereby a 100% success rate was achieved for obtaining high ion counts (>10,000), high resolution (>500), high signal to noise ratio (>1 to 5000) and shorter acquisition time (˜90 s/1000 laser shot spectra) with spectra selection criteria set to a minimum signal intensity of 10,000, a maximum signal intensity to 64,000 and the minimum resolution set to 500. These criteria and outcomes are significant above previous experience described for the MALDI-ToF mass spectrometric analysis of tryptic peptides. The main advantages of the new spotting method against the previously used dried droplet sample spotting method was high spot-to-spot reproducibility, the requirement for less matrix and obviously the elimination of the step of premixing the sample with matrix.

### 1.1 Trial of New Sample Handling/Collection Method

In this sample handling/collection method, 0.5 μl samples were directly added to 49.5 μl of buffer (50 mM ammonium bicarbonate, 2 mM CaCl₂, pH 8.3) with a resulting dilution factor of 1:100. The MALDI-TOF mass spectrometric analysis of the samples in the linear mode in the 7000-17000 m/z range show that the single charged [M+H]⁺ and double charged [M+2H]⁺ Hb A α and β chains were resolved with a high mass accuracy and with an inherent error less than 1 Da for single charged α and β chains, as depicted in Table 4. The corresponding MALDI-TOF mass spectrum is shown in FIG. 68. The blood samples diluted directly into the buffer were stored at 4° C. and subjected to repeated MALDI-TOF mass spectrometric analysis. The results had a ˜100% reproducibility. The MALDI-TOF MS analyses with this ‘on-carrier’ tryptic digestion procedure of the samples are described elsewhere in the patent application.

TABLE 4
Resolved m/z values of intact α and β chains, single and
double charged, using MALDI-ToF mass spectrometric analysis
in the linear mode.

| | Theoretical | Received | Error |
|---|---|---|---|
| | m/z values | m/z values | m/z value |
| Doubly charged α | 7568.19 | 7572.08 | −3.89 |
| Doubly charged β | 7934.61 | 7941.90 | −7.29 |
| Singly Charged α | 15127.37 | 15126.88 | −0.49 |
| Singly Charged β | 15868.23 | 15868.03 | −0.20 |

### 1.3 Optimising Hb Sample Dilution for MALDI-ToF Mass Spectrometric Analysis with the New Procedures:

Although good spectra were obtained for the 1:100, 1:1000 and 1:10000 dilutions, the method was developed for the 1:100 dilution instead of the 1:1000 dilution, since this is a convenient dilution factor for other researchers, and because in the MALDI-TOF mass spectra of Hb tryptic peptides some peptides appeared to be have a low ion current abundance at the level of 1:1000 dilution. The low ion abundance may result in these peptides being resolved with a lower mass accuracy and thus be unsuited for automated data analysis. The trade-off at higher sample concentration is the appearance of peaks derived from other blood proteins. Although these additional peaks complicate to a minor extent the spectral data analysis, requiring extra care for interpreting the accumulated mass spectra, they do provide additional information since their presence was found to correlate with the conditions employed for the sample preparation, kinetics of enzyme digestion, digestion time, etc, thus enabling these non-Hb associated peaks to be used as “internal standards” for the detection other Hb chains within the sample with an abundance of >2% in relation to the α- and β-chains.

The concentrations of the unpurified blood samples were varied in order to optimise the selection of the dilution factor. Blood diluted with either with 50 mM ammonium bicarbonate buffer, 2 mM CaCl₂, pH 8.3, or with deionised water gave similar results for all dilution factors when the reversed two layer sample spotting method using a sample to matrix ratio of 2:1 (sample 1 μl, matrix 0.5 μl) was employed. This outcome was not observed when other types of matrix compounds, such as α-CHCA and 2,5-DHB, were used. The 1 to 10 dilution of blood produced a non-homogenous sample spot. This also resulted in a very weak signal for both the α and β chain with no or a very poor separation of the matrix adducts of the chains, as shown in FIG. 69. The signal to noise ratio observed for the 1 to 10 dilution of blood was 126.38 (SD 133.34), as shown in Table 5, obtained from 10 consecutive spectra collected using the manual mode of data acquisition with a laser intensity of 2700. Although increased laser intensity gave a slightly better result, the m/z values varied to a great extent. Lower laser intensity produced no or a very poor signal of either chain, and the abundance of ions were very low for this particular concentration.

TABLE 5
Resolution and signal/noise ratio of spectra of intact globin chains
at different dilutions, spotted with the reversed two layer method,
whereby SA was used as a matrix.

| | Globin | Signal to Noise | Resolution |
|---|---|---|---|
| Dilution | Chain | Ratio Mean (SD) | Mean (SD) |
| 10 | α | 126.38 (133.34) | X |
| 100 | α | 6961.76 (544.31) | 551.2 (47.54) |
| 1000 | α | 6567.19 (521.12) | 568.22 (52.37) |
| 10000 | α | 6908.58 (576.91) | 641.7 (60.01) |
| 10 | β | 35.5 (27.62) | X |
| 100 | β | 3671.29 (551.26) | 523 (72.11) |
| 1000 | β | 3497.39 (558.1) | 526 (57.34) |
| 10000 | β | 2363.58 (278.93) | 599.9 (51.94) |

Sample: Whole EDTA treated blood, at different dilutions.
Number of spectra: 10, each spectrum is accumulation of 10 spectra.

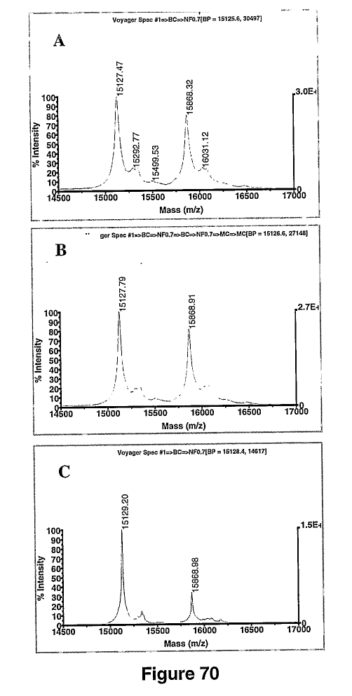

Excellent MALDI ToF mass spectra were obtained for the 1:100, 1:1000, and 1:10,000 dilutions for the un-purified EDTA-treated blood in the linear mode, as shown in FIGS. 70, A, B and C respectively, with good separation also of the associated matrix adducts. The resolution and signal-to-noise ratio obtained for these dilutions were similar, above 500 and 6000 respectively in each case, with good reproducibility, as depicted in Table 6. Resolution and S/N ratio data were obtained from at least 10 consecutive spectra, whilst each spectrum was obtained with 100 laser shots. Remarkable improvements of mass resolution and signal-to-noise ratios was obtained, as depicted in Table 6, by accumulating 10 consecutive spectra, whereby each spectrum was obtained with 100 laser shots, when compared with 5 accumulated spectra, each consisting of 100 laser shots, as depicted in Table 7, for dilutions 1:100 and 1:1000. For both dilutions, there was a two-fold increase in resolution while the signal-to-noise ratio improved by nearly 2000 fold.

The mass accuracy obtained for the dilutions 1:100, 1:1000, and 1:10,000 were persistently within 0.01%. The ion count was consistently above 10,000 for these dilutions, as shown in Table 7.

TABLE 6
Resolution and S/N ratio of whole human blood spectra for
SA as matrix spotted with the reversed two layer method.

| Chain | Dilution | Signal-to-Noise Ratio(SD) | Resolution(SD) |
|---|---|---|---|
| α | 1 to 1000 | 4580.73(829.13) | 330.67(69.33) |
| β | 1 to 1000 | 3769(528.78) | 297.67(5.68) |
| α | 1 to 100 | 4566.861(2673.36) | 278.75(101.43) |
| β | 1 to 100 | 2273.15(501.67) | 201(53.25) |

Number of spectra: 10, each spectra is accumulation of 5 spectra.

TABLE 7
Obtained ion counts of whole human blood spectra at different
dilutions for SA as matrix spotted with the reversed two layer method.

| Dilution | Mean ion count | Std. Deviation |
|---|---|---|
| 10 | 3.36E+03 | 7.31E+02 |
| 100 | 2.67E+04 | 6.02E+03 |
| 1000 | 2.22E+04 | 3.88E+03 |
| 10000 | 1.81E+04 | 3.63E+03 |

Number of spectra: 10, each spectrum is accumulation of 5 spectra

### Example 2

### Proteolytic Digestion Methods

To find the best digestion conditions for human Hb α and β chains, and to assess the sequence coverage for both the chains and to document their proteolytic fragmentation pattern a time course proteolytic digest experiments on Hb A standard followed by whole EDTA treated diluted blood, normal Hb A and variant Hb E, were performed. Initially, in solution digests were performed followed by on carrier experiments to devise a rapid on carrier proteolytic digestion method with a novel degradable detergent. The optimised on carrier digest method was subsequently tested with some known and unknown variants, and with other proteolytic enzymes.

### 2.1 Solution Phase Tryptic Digestion

### 2.1.1 HbA Standard

To optimise the digestion time and sequence coverage of the globin chains a time course experiment on Hb A standard was performed. 9 ml of the dissolved Hb A standard were incubated in a water bath at 37° C. for 5 minutes before adding 1 ml of a 10-fold trypsin stock solution. The final molar ratio of trypsin to Hb was 1:10. The 10 ml trypsin Hb solution was incubated at 37° C. in a water bath to allow the digestion process to occur. Aliquots of 250 μl were taken at time points 2, 4, 5, 6, 8, 10, 12 15, 20, 30, 45, 60 min and 2, 4, 8 and 24 hours and the digest was stopped with 62.5 μl 10% TFA (trifluoro acetic acid) yielding 83.7 μM Hb with a final concentration of 2% TFA for each time point. The samples were further diluted 1:5 with ACN:H₂O (50:50) (v:v), 0.1% TFA for MS analysis. The samples were spotted with the two-layer technique by successive spotting 2 μl of either the SA or alternatively α-CHCA and 1 μl of sample. The final Hb concentration on the sample plate for each spot is 16.7 pmol/μl.

### 2.1.2 Hb in Whole Human Blood

The first step in optimising the analysis of the whole human EDTA treated blood sample was to carry out a similar time course in solution tryptic digest experiment as for the Hb standard to document the fragmentation pattern and sequence coverage. In addition, applicability of the surfactant RapiGest™ (sodium 3-[(2-methyl-2-undecyl-1,3-dioxolan-4yl)methoxy]-1-propane-sulfonate) was investigated to enhance the efficiency of the proteolytic digest and to decrease the digest time. In this experiment, EDTA-treated whole human blood with an approximate Hb concentration of 9.3 mM (150 mg/mL) was diluted 1:100 (v/v) with 50 mM ammonium bicarbonate buffer, 2 mM CaCl₂, pH 8.3. The diluted blood was subjected to a tryptic digest with and without a surfactant. For the digest without the surfactant 95 μl of blood and for the surfactant aided digest 90 μl of diluted blood was incubated with 5 μl 2% stock solution of the surfactant sodium 3-[(2-methyl-2-undecyl-1,3-dioxolan-4yl)methoxy]-1-propane-sulfonate in 50 mM ammonium bicarbonate buffer, 2 mM CaCl₂, pH 8.3 at 37° C. in a water bath for 5 minutes. Then the digest was started by adding 5 μl of a 20-fold diluted trypsin stock solution (1.3 mg/ml) in 50 mM ammonium bicarbonate buffer, 2 mM CaCl₂, pH 8.3 to both the samples to attain a final molar ratio of trypsin to Hb of 1:34. Both the samples were kept incubated at 37° C. to continue the digestion reaction and 10 μl aliquots were taken at different time points starting from 15 min and then 30 min, 1 h, 1 h 30 min, 2-8 hours in 1 h intervals and the last one at 24 hours. The digests were stopped by adding 2.5 μl 10% TFA to the aliquot of each time point yielding a final TFA concentration of 2%. For MS analysis the samples were then diluted 1:10 with ACN:H₂O (50:50) (v:v), 0.1% TFA.

### 2.2 On carrier Digestion

To optimise and develop a rapid, simple, robust proteolytic digestion method the following on carrier experiments were carried out using surfactant, initially with trypsin followed by independent experiments with endoproteinase Glu C and Asp N on whole normal blood and blood containing Hb variants.

### 2.2.1 Tryptic Digestion of Hb in Whole Human Blood

2 μl of 20-fold trypsin stock solution with a trypsin concentration of 1.3 mg/ml (54.5 μM) equalling 5.45 pmole/μl was spotted for each digest on the sample plate and air dried at room temperature (22° C.) for 5 minutes. The sample plate was then incubated for 15 min at 37° C. and placed on a heating block or heating plate at 37° C. for 5 minutes before applying the sample. For on carrier tryptic digest of whole EDTA treated human blood, 19 μl of blood sample, diluted 1:100 with 50 mM ammonium bicarbonate buffer, 2 mM CaCl₂, pH 8.3, was incubated either at 100° C. or at 37° C. for 5 min with 1 μl of 2% (w/v) of the surfactant sodium 3-[(2-methyl-2-undecyl-1,3-dioxolan-4yl)methoxy]-1-propane-sulfonate in 50 mM ammonium bicarbonate buffer, 2 mM CaCl₂, pH 8.3. The sample incubated at 100° C. was cooled to 37° C. before adding the sample to the enzyme. 1 μl of this heat-denatured sample (93 μM Hb=93 pmole/μl) was spotted on the dried trypsin spots yielding a final molar ratio of trypsin to Hb for each spot of 1:17. The digestion reaction was stooped with 0.5 μl 10% TFA after 2 s, 10 s to 1 min at 10 s intervals, and then onwards to 3 min at 15 s intervals. Matrix, 0.5 μl of SA was added and the samples air-dried.

### 2.2.2 Endoproteinase Glu C Digestion of Hb in Whole Human Blood

The optimised time for on carrier tryptic digestions in the presence of the surfactant sodium 3-[(2-methyl-2-undecyl-1,3-dioxolan-4yl)methoxy]-1-propane-sulfonate was then tested using the proteolytic enzyme Glu C. For the on carrier digestion of Hb in whole EDTA treated blood endoproteinase Glu C stock solution was made by dissolving 25 μg of lypophilized Glu C in 25 μl of 50 mM ammonium bicarbonate buffer, 2 mM CaCl₂, pH 8.3. Then 1.5 μl of a further 50 fold diluted Glu C stock solution (1 μg/μl=34.5 μM) in 50 mM ammonium bicarbonate buffer, 2 mM CaCl₂, pH 8.3 equalling 0.69 μM/μl was spotted for each digest on the sample plate and air dried at room temperature. 19 μl of blood sample, diluted 1:100 with 50 mM ammonium bicarbonate buffer, 2 mM CaCl₂, pH 8.3, was incubated for 5 min with 1 μl of 2% (w/v) sodium 3-[(2-methyl-2-undecyl-1,3-dioxolan-4yl)methoxy]-1-propane-sulfonate in 50 mM ammonium bicarbonate buffer, 2 mM CaCl₂, pH 8.3, at 37° C. After the heat denaturation step, before adding the sample to the enzyme, the sample plate was placed on a heating plate at 37° C. for 5 minutes. 1 μl of this blood sample was spotted on each of the dried Glu C spots yielding a final molar ratio of Glu C to Hb of 1:90 and stooped with 0.5 μl 10% TFA after 3 min. The samples were allowed to dry before 0.5 μl of SA was added.

### Example 3

### MALDI-ToF MS Analysis: Intact Hb a in Whole Blood

### Identification of a and β Chains and Their Adducts

### Globin Chain Peaks of Human Hb A

The MALDI-TOF mass spectrum derived in the linear mode in the 5000-25000 m/z range for unpurified whole EDTA treated human blood containing Hb A (α₂β₂) show the double charged m/z values (received [M+2H]++/2: 7596.23 and 7959.33 (expected 7568.19 and 7934.61), the single charged m/z values (received [M+H]+: 15127.47 and 15868.31, expected 15868.23) and the m/z values for the α- [symbol] ,α- [symbol] ,β-β dimers (received [M+H]+: 30173.07, 30914.66 and 31677.26) as shown in Fig. I. The m/z values of the single charged intact [symbol] chain and β chain of Hb A were measured with an error of 0.10 and 0.08 Dalton respectively. Errors associated with other peaks are listed in Table 8.

TABLE 8
Mass accuracy of obtained peaks in the linear mode for
monomeric and dimeric globin chains in Dalton.

| | Theoretical | Received | Error in |
|---|---|---|---|
| | m/z values | m/z values | m/z value |
| Double charged α | 7568.19 | 7596.2338 | 32.04 |
| Double charged β | 7934.61 | 7959.3346 | 24.71 |
| Single charged α | 15127.37 | 15127.47 | 0.10^{a} |
| Single charged β | 15868.23 | 15868.31 | 0.08^{b} |
| α-α | 30254.74 | 30173.07 | 81.67 |
| α-β | 30995.6 | 30914.66 | 80.94 |
| β-β | 31736.46 | 31677.26 | 59.2 |

Δppm:
^{a}6.61,
^{b}5.04.

### Adducts

The masses of 15333.37 and 16078.54 with their respective mass differences of the received single charged α and β mass of 205.9 and 210.23 are considered to derive from Hb-SA adducts. Hb matrix adducts were also reported previously. The masses of 15292.81 and 16031.27 with their respective mass differences from the received single charged α and β mass of 165.3 and 163.0 are considered to derive from glycation of the respective chains, this finding is in agreement with previous reports. Errors associated with the peaks are listed in Table 9.

TABLE 9
Mass accuracy of obtained peaks in the linear mode for glycated α and
β chains, as well as SA adducts of both the chains.

| | | Received | |
|---|---|---|---|
| | Theoretical | average | Error in |
| | average mass | mass | m/z value |
| Glucose adduct | 162.1424 | | |
| Glycated α | 15289.51 | 15292.81 | −3.30 |
| Glycated β | 16030.37 | 16031.27 | −0.90 |
| Molecular weight of SA | 224.07 | | |
| Dehydroxy (—OH) SA | 207.06 | | |
| adduct | | | |
| SA adduct α | 15334.43 | 15333.37 | 1.06 |
| SA adduct β | 16075.29 | 16078.54 | −3.25 |

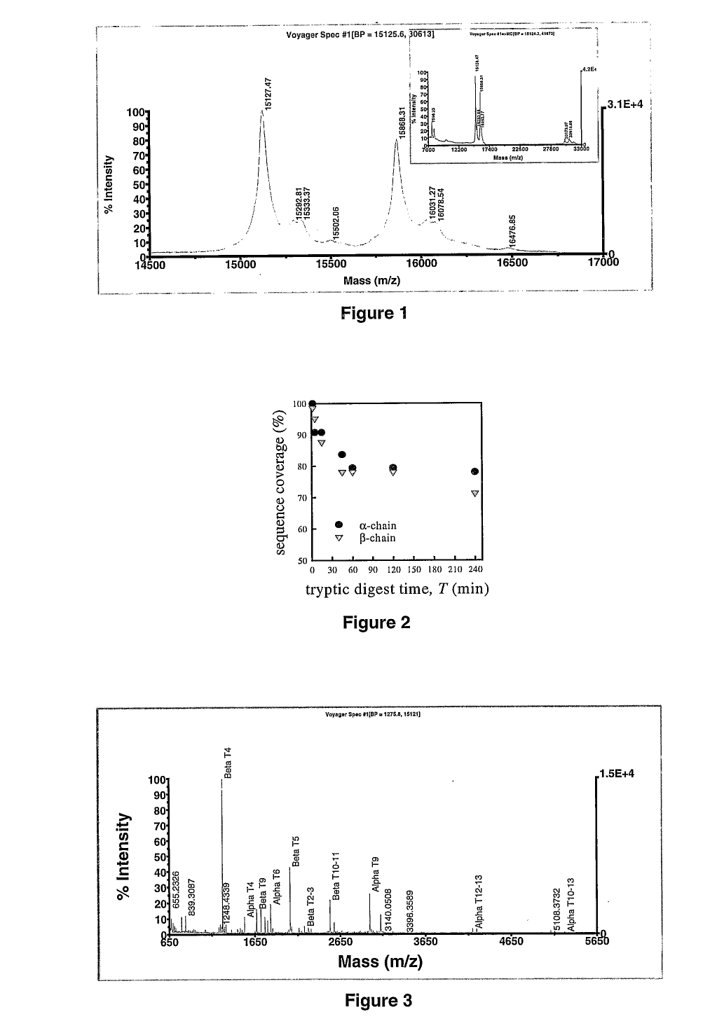

Relevant spectra: FIG. 1.

### Example 4

### MALDI-ToF MS Analysis: Optimisation of Proteolytic Digestion

### Free Solution Phase Tryptic Digestion

### Hb A Standard

Initial experiments were designed to establish the time necessary to achieve a complete Hb standard digest followed by a time course experiment to document the sequence coverage of the respective globin chains at different time points using the enzyme trypsin. The sample procedure was that outlined in example 2.1.1. A complete digest was obtained after 24 hours, as judged by the disappearance of the Hb chains in the corresponding reversed-phase HPLC chromatograms (data not shown) and the MALDI-TOF mass spectra in the m/z range from 5000-25000 in the linear mode (data not shown). The time course of the free solution digests of the Hb A standard versus the sequence coverage is depicted in FIG. 2. A sequence coverage of 87.94% for the α chain and 75.34% for the βchain was obtained from the 24 h digest products of Hb standard. In a complete digest of haemoglobin using trypsin 14 peptides for the α chain and 15 peptides for the β chain can be produced. Theoretically a complete digest would correspond to a 87.23% sequence coverage for the α chain and 93.84% sequence coverage for the βchain in the 650-5650 m/z window, which was not achieved for the 24 h digest. The missing fragments were αT5, αT7, αT10, αT13 and βT4, βT13, βT14, βT15. It was observed, as shown in FIG. 2, that the sequence coverage for both, the α and βchain increased with shorter digest time, due to missed cleavage sites at a similar rate for both chains. A sequence coverage of 98.58% for the α chain and 98.63% for the β chain was obtained for a 2 min digest. The small fragments of the α chain, αT2, αT3, αT7, αT8, αT10, and β chain, βT6, βT7, βT8, were successfully captured, whereby only the dipeptides αT14 and βT6 were lost. The corresponding mass spectrum is shown in FIG. 3. The calculation of the accessible surface area of the enzyme recognition residues Lys and Arg on human Hb A (α₂β₂) range for the α chain from 3.8 to 164.3 Å² for the β chain from 2.6 to 169.2 Å² with small differences for identical chains within the tetramer. For both the fragments which were not captured, the respective trypsin recognition residues within the Hb chains are well surface exposed, with an accessible surface area of 75-80 Å² for Lys¹³⁹ for the C-terminal αT14 and of 108-112 Å² for Lys⁵⁹ and 96-110 Å² for Lys⁶¹ for the internal βT6, which makes an early cleavage likely. The identified 12 peptides within the 10 ppm mass accuracy window were, with increasing mass, αT4, αT6, αT9, αT8-9, and βT4, βT3, βT9, βT12, βT8-9, βT5, βT2-3, and βT10-11 as shown in Table 10.

TABLE 10
Mass accuracy of obtained tryptic peptides derived from a
2 min free solution digest of Hb standard in the reflector mode.

| | Mass matched | | | |
|---|---|---|---|---|
| m/z submitted | [m + H]⁺ | Δppm | Position | Fragments |
| 1529.74 | 1529.73 | 2.81 | 17-31 | αT4 |
| 1833.89 | 1833.89 | 0.41 | 41-56 | αT6 |
| 2996.48 | 2996.49 | −3.26 | 62-90 | αT9 |
| 3124.58 | 3124.59 | −1.55 | 61-90 | αT8-9 |
| 1274.72 | 1274.73 | −6.82 | 31-40 | βT4 |
| 1314.67 | 1314.67 | 0.03 | 18-30 | βT3 |
| 1669.9 | 1669.89 | 4.49 | 67-82 | βT9 |
| 1719.97 | 1719.97 | 0.16 | 105-120 | βT12 |
| 1797.99 | 1797.99 | 0.26 | 66-82 | βT8-9 |
| 2058.95 | 2058.95 | 2.42 | 41-59 | βT5 |
| 2228.16 | 2228.17 | −4.68 | 7-30 | βT2-3 |
| 2529.22 | 2529.22 | 1.87 | 83-104 | βT10-11 |

### 4.2 Proteolytic Digestion Using a Degradable Surfactant Haemoglobin A in Whole Unpurified Human Blood.

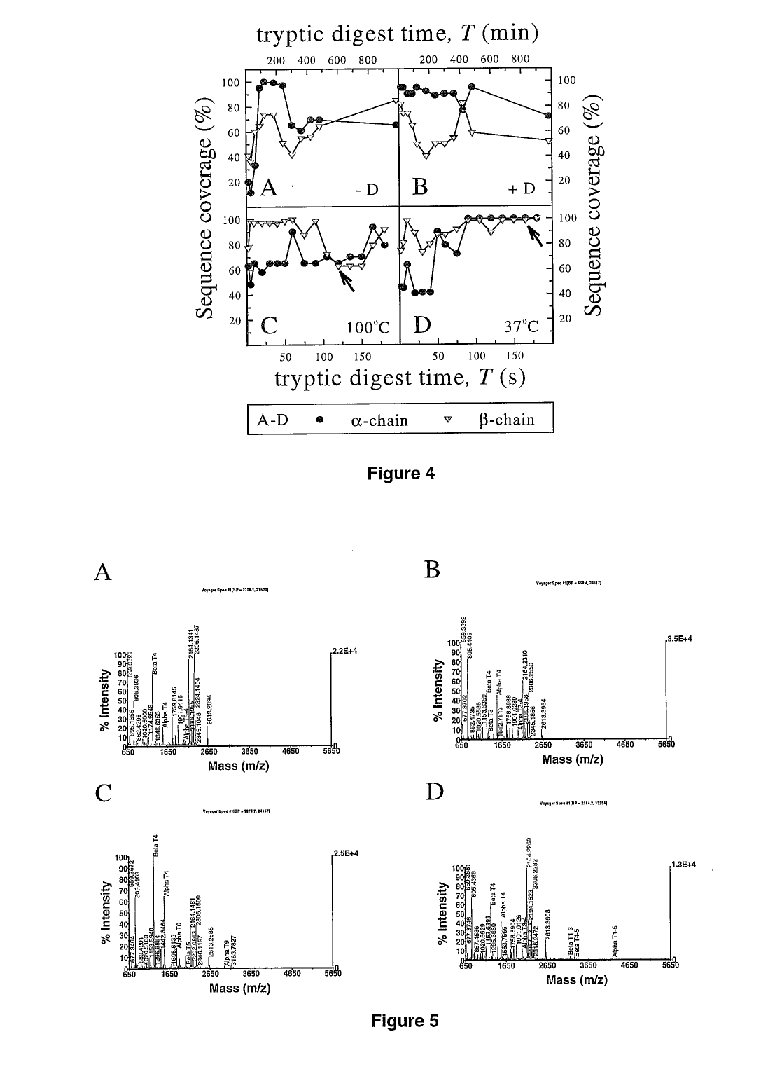

The effect of the ionic surfactant sodium 3-[(2-methyl-2-undecyl-1,3-dioxolan-4yl)-methoxy]-1-propane-sulfonate (RapiGest™ SF) on the sequence coverage of the Hb A α- and β-chain in a free solution digest in 1:100 diluted EDTA treated blood was investigated by performing a time course experiment. The procedure followed was that of example 2.1.2. The results for the individual digest times in the absence and the presence of the surfactant are depicted in FIG. 4, Panel A and B, respectively. Without a surfactant, in free solution digest, as shown in Panel A, within 24 hours, a substantial cleavage was obtained, with a sequence coverage of 62.5% for the α-chain and 84.93% for the β chain. Generally, αT12, αT13, βT10 and βT12 are believed to precipitate during the tryptic digest. In this experiment, the missing fragments were αT12, αT13, αT14 and βT6, βT7, βT12; except for the one-hour time point where the βT10 was detected. With shortened cleavage time both the curves for the α and the β chains, the sequence coverage went through a relative minimum with a coincidental optimal digest time in the range between 90-240 min. There was a significant drop of sequence coverage when the digestion time is less than 90 min. The optimum cleavage time is 2 hours, when a 100% sequence coverage was obtained for the α-chain and 73.97% for the β chain. The missing “chain fragments were βT9, βT10, βT11. Interestingly, it was noted that, besides lower sequence coverage in blood, the missing fragments were all different, except αT13 when the free solution digest of the Hb A standard and Hb A in blood was compared.

With the surfactant RapiGest™ in a free solution digest, as shown in Panel B, a good sequence coverage was obtained for Hb A digestion times below two hours, with the excellent cleavage time of 15 min and a sequence coverage of 95.04% for the α chain and 82.19% for the β chain. Here, the fragments αT11 and βT13-15 were missing. Interestingly, at 420 min, the occurrence of βT10-13 coincides with the disappearance of αT12-14, as if these fragments would compete for ionisation and desorption. None of the fragments believed to precipitate, αT12, αT13, βT10 and βT12 could be detected. Although the occurrence of surfactant dimer and trimer formation with m/z values of 855.617 and 1271.922, respectively, was reported, only the dimer, with a m/z value of 855.617, was identified infrequently, under the conditions employed.

### 4.3. Proteolytic on Carrier Digestion Using the Novel Degradable Surfactant

### Proteolytic Enzyme—Trypsin

### Hb a in Whole Unpurified Human Blood

The sample was prepared according to the general procedure outlined in example 2.2.1. The sequence coverage of the Hb α and β chain in 1:100 diluted EDTA treated blood for an on carrier solution digest in the presence of the surfactant RapiGest™ SF after a 100° C. or 37° C. pre incubation is plotted in FIG. 4, Panel C and D, respectively. With the combined effects of heat and surfactant denaturation on the proteolytic digest after a pre-incubation at 100° C., as shown in Panel C, the highest sequence coverage was obtained in the region from 10 s to 60 s, with the α chain sequence coverage fluctuating, whilst the β chain sequence coverage showing a plateau. Although the sequence coverage was highest at 60 s, with 90.07% for the α chain and 100% for the β chain, the high sequence coverage of the α chain was due to the rare occurrence of αT12 in this particular time course experiment (which only occurred at the 2 s and the 60 s time points).

For the on carrier digest at 37° C. in the presence of the surfactant, as shown in Panel D, the sequence coverage for both the chains was consistently high with a plateau between 90 s and 180 s. Detection of the αT12-14 explains the obtained 100% sequence coverage of the αchain within the plateau, which did not appear in shorter digestion times. For the β chain at each time point of the plateau, one fragment was missing, whereby the absence of the large βT12 fragment (16 amino acid) at 2 min had the highest impact, whilst all the other missing fragments were dipeptides, either βT6 or βT15. Complete sequence coverage was obtained for both, the α and the β chain, at 180 s. With these particular conditions, from 90-180 s, method robustness was achieved, i.e. where small changes in digestion time result in only small changes in sequence coverage. If the results from both on carrier experiments, the combined effects of heat plus surfactant denaturation for a pre-incubation at 100° C. and the surfactant denaturation alone (with the their plateaus from 10-60 s and 90-180 s, respectively) is analysed, it is obvious that the surfactant alone only partially denatures the proteins, whilst the additional heat increases the denaturation and thus the accessibility of additional cleavage sites.

The mass spectra corresponding to selected time points 10 s, 30 s, 90 s and 180 s in the on carrier digest at 37° C., are shown for the m/z range from 650-5600 in FIG. 5, Panel A-D. For the tryptic peptide spectra, as shown in FIG. 5, Panel A-D, in particular αT4 and βT4, were typical Hb signature peptides due to their signal intensity and nearly ubiquitous appearance as single peptides in the MS spectra, except at the 2 s time points, where they were part of a peptide with at least one missed cleavage site.

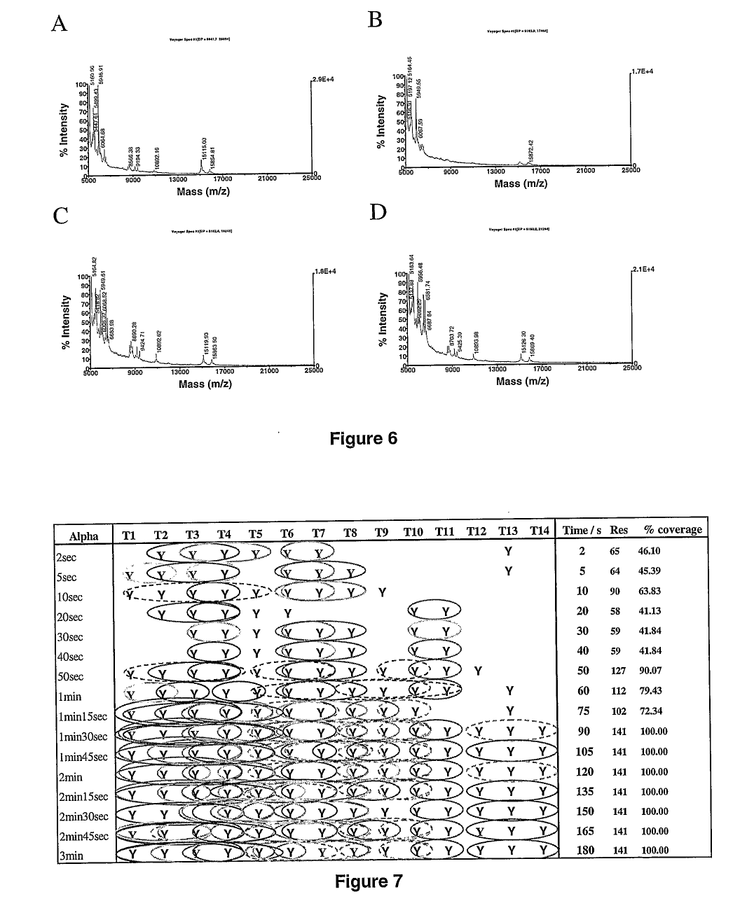

To additionally monitor the digest from the disappearance of the intact globin chains, mass spectra corresponding to each time point were obtained in linear mode. FIG. 6, Panel A-D corresponds to the selected time points 10 s, 30 s, 90 s and 180 s in the on carrier digest at 37° C., shown for the m/z range from 5000-25000. The spectra obtained in the linear mode, as shown in FIG. 6, Panel A-D, reveals that at the selected time points only low amounts of intact Hb α and β chains are still present, although their abundance decrease as digestion time is increased. The appearance of peaks below m/z 11000 Da signifies the digestion activities.

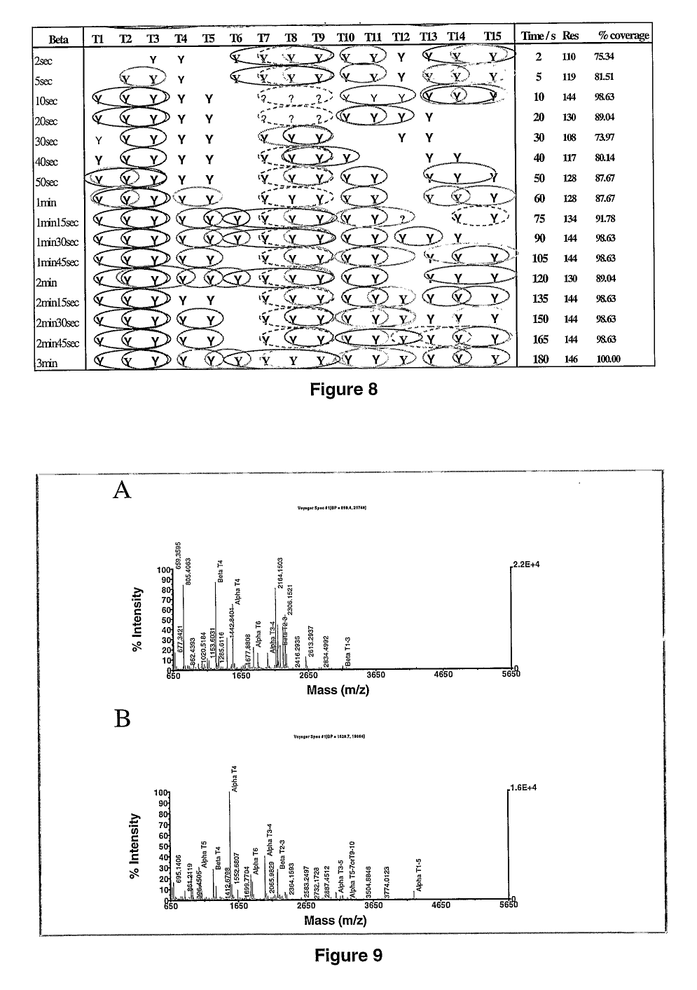

The spectrum in FIG. 5, Panel D, which yielded complete sequence coverage, shows the occurrence of the bigger fragments, βT1-3, βT4-5, and αT1-5, which are typical for an incomplete Hb digest. It is however the consistent occurrence of α12-15 for the α chain, shown in FIG. 7, and the capture of the dipeptide βT6, either as βT5-6 or as βT6-9 for the β chain, as shown in FIG. 8, which was crucial for a 100% sequence coverage of both chains. FIG. 7 and FIG. 8, illustrates the peptide fragmentation patterns for on carrier tryptic digestion at different time points in the presence of the surfactant, RapiGest™ SF. For the spectrum depicted in FIG. 5, Panel D, with complete sequence coverage for both the globin chains of haemoglobin A (α₂β₂), 9 fragments were detected within the 10 ppm window. The peptides within 10 ppm window were, with increasing mass, αT5, αT4, αT6, αT3-4, αT6-7 and βT4, βT3, βT2-3, and βT1-3, as shown in Table 11.

All other Hb A tryptic fragments had a mass accuracy below 10 ppm and were not used in the computational identification procedure. In addition to the tryptic fragments of α and β globin chains of Hb A, 5 fragments of the δ-chain were identified. The δ-chain is homolog to the β-chain differing in 10 amino acids, one of which is, Arg¹¹⁶, resulting in an additional trypsin cleavage site. Since HbA₂ (α₂δ₂) constitutes only less than 3% of the hemoglobins, the abundance of these peptides and consequently their mass accuracy was quite low, as shown in Table 12. Nevertheless, the method was considered able to detect aberrant high Hb abundances of HbA₂ (α₂δ₂). At this stage and with the set conditions, no γ chain fragments from Hb F (α₂γ₂), present in very low abundance in normal human adult blood (<1%) were detected.

TABLE 11
Mass accuracy of obtained peaks derived from in solution digest
of α and β Hb chains, at the 3 min time point, with trypsin in the
presence of RapiGest ™, in reflector mode, analysed by the
Protein Prospector software.

| | Mass Matched | | | |
|---|---|---|---|---|
| m/z submitted | [m + H]⁺ | Δppm | Position | Fragments |
| 1071.5574 | 1071.5549 | 2.4 | 32-40 | αT5 |
| 1529.7278 | 1529.7348 | −4.6 | 17-31 | αT4 |
| 1833.8961 | 1833.8924 | 2 | 41-56 | αT6 |
| 2043.0033 | 2043.0048 | −0.8 | 12-31 | αT3-4 |
| 2213.0914 | 2213.0892 | 1 | 41-60 | αT6-7 |
| 1274.7216 | 1274.7261 | −3.5 | 31-40 | βT4 |
| 1314.6607 | 1314.6654 | −3.6 | 18-30 | βT3 |
| 2228.1716 | 2228.1675 | 1.8 | 7-30 | βT2-3 |
| 3161.6639 | 3161.6595 | 1.4 | 1-30 | βT1-3 |

This table corresponds to the MALDI-ToF mass spectrum depicted in FIG. 5, Panel D.

TABLE 12
Obtained peaks derived from the δ chain obtained from 3 min
digest with trypsin in presence of RapiGest ™, in the MALDI-ToF
reflector mode, analysed by the Protein Prospector software.

| | Mass matched | | | |
|---|---|---|---|---|
| m/z submitted | [m + H]⁺ | Δppm | Position | Fragments |
| 1256.1469 | 1256.6593 | 407.9 | 18-30 | δT3 |
| 2197.7423 | 2197.1723 | 259.4 | 7-30 | δT2-3 |
| 3019.4325 | 3018.5618 | 288.5 | 117-144 | δT12-15 |

This table corresponds to the MALDI-ToF mass spectrum depicted in FIG. 5, Panel D.

In the methods of the invention, autocatalytic tryptic fragments very rarely detected with low abundance or peak intensity, not surprisingly, firstly because of the shortness of the digest time, only 3 min, and secondly because of the inactivity of potentially present enzymes present (like serine-proteases), caused by the denaturing action of the surfactant. As the trypsin activity is maintained, it was anticipated that the trypsin concentration could be further decreased, leading to substantial cost savings in high-throughput applications. Moreover, the surfactant could increase the lifetime of the expensive enzyme-linked sample plates.

### 4.4 Verification of Method Robustness: Whole Blood with Varied Concentration

An on carrier digest, at 37° C., of two different dilutions of unpurified human blood with ammonium bicarbonate, 1:100 and 1:10, was performed with the presence of the ionic surfactant. The digests were stopped each at 2 min, and the obtained spectra of the digested blood sample for the two dilutions were compared. For the 1:100 dilutions, the sequence coverage for the α chain was 100% and for the β chain was 89.04% due the missing of βT12, as shown in FIG. 9-A. For 9-B the number of Hb A (α₂β₂) fragments detected in the 1:100 dilution digest spectrum was lower than the number fragments detected in the 1:10 dilution digest spectrum, both within the 10 ppm window. The nine Hb A (α₂β₂) fragments detected in the 1:100 dilution digests were, with increasing mass, αT5, αT4, αT6, αT3-4, αT6-7 and βT4, βT3, βT2-3, βT1-3, as shown in Table 13.

TABLE 13
Mass accuracy of the obtained peaks of the α and the β chains
derived from an on carrier digestion of whole blood, 1:100 dilution,
at the 2 min time point, with trypsin in the presence of RapiGest ™,
in the reflector mode, analysed by the Protein Prospector software.

| | Mass matched | | | |
|---|---|---|---|---|
| m/z submitted | [m + H]⁺ | Δppm | Position | Fragments |
| 1071.5528 | 1071.5549 | −1.97 | 32-40 | αT5 |
| 1529.7355 | 1529.7348 | 0.41 | 17-31 | αT4 |
| 1833.9039 | 1833.8924 | 6.24 | 41-56 | αT6 |
| 2042.9983 | 2043.0048 | −3.19 | 12-31 | αT3-4 |
| 2213.0841 | 2213.0892 | −2.29 | 41-60 | αT6-7 |
| 2341.1860 | 2341.1842 | 0.77 | 41-61 | αT6-8 |
| 1274.7294 | 1274.7261 | 2.59 | 31-40 | βT4 |
| 1314.6623 | 1314.6654 | −2.37 | 18-30 | βT3 |
| 2228.1814 | 2228.1675 | 6.22 | 7-30 | βT2-3 |

This table corresponds to the MALI-ToF mass spectrum depicted in FIG. 9-B.

However, the thirteen Hb A (α₂β₂) fragments that were detected in the 1:10 dilution in the 10 ppm window, with increasing mass were, αT4, αT5, αT6, αT3-4, αT6-7, αT6-8, αT3-5, αT1-4, αT1-5, and βT4, βT3, βT2-3, βT1-3, as shown in Table 14, indicate that with increasing blood concentration the number of peaks detected with lower than 10 ppm mass accuracy increase.

TABLE 14
Mass accuracy of the obtained peaks of the α and the β chains
derived from an on carrier digestion of whole unpurified blood sample,
1:10 dilution, at the 2 min time point, with trypsin in the presence of
RapiGest ™, in the reflector mode, analysed by the Protein
Prospector software.

| | Mass matched | | | Tryptic |
|---|---|---|---|---|
| m/z submitted | [m + H]⁺ | Δppm | Position | fragments |
| 1071.547 | 1071.55 | −7.1 | 32-40 | αT5 |
| 1529.735 | 1529.73 | −0.08 | 17-31 | αT4 |
| 1833.901 | 1833.89 | 4.83 | 41-56 | αT6 |
| 2043.004 | 2043 | −0.45 | 12-31 | αT3-4 |
| 2213.085 | 2213.09 | −1.77 | 41-60 | αT6-7 |
| 2341.186 | 2341.18 | 0.85 | 41-61 | αT6-8 |
| 3095.527 | 3095.54 | −4.53 | 12-40 | αT3-5 |
| 3195.649 | 3195.66 | −1.93 | 1-31 | αT1-4 |
| 4248.206 | 4248.19 | 3.34 | 1-40 | αT1-5 |
| 1274.727 | 1274.73 | 0.91 | 31-40 | βT4 |
| 1314.661 | 1314.67 | −3.12 | 18-30 | βT3 |
| 2228.17 | 2228.17 | 1 | 9-30 | βT2-3 |
| 3161.688 | 3161.66 | 8.93 | 1-30 | βT1-3 |

This table corresponds to the MALDI-ToF mass spectrum depicted in FIG. 9-A.

### Investigation of the Compatibility of the Proteolytic Enzyme Glu C with the Novel Surfactant

### Hb A in Whole Unpurified Human Blood

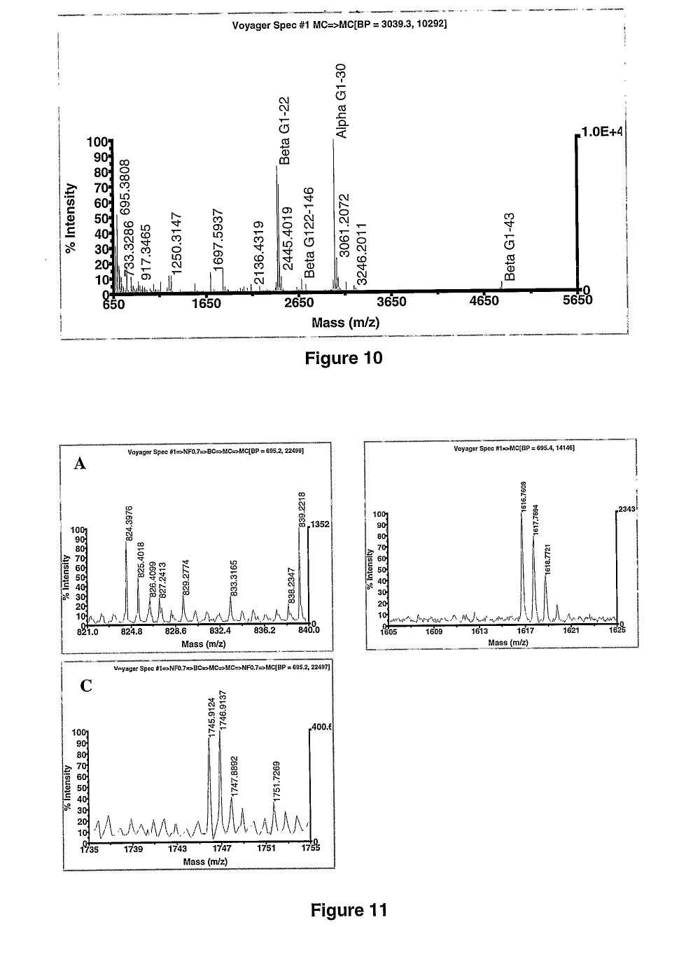

The sequence coverage and fragmentation pattern of the Hb α and βchains in EDTA treated unpurified whole human blood, diluted 1:100 in ammonium bicarbonate, for an on carrier 3 min solution digest with endoproteinase Glu C in the presence of the surfactant RapiGest™ SF after 37° C. pre incubation was investigated. This followed the general procedure outlined in example 2.2.2. From a theoretical standpoint, a complete digest, which produces 5 α and 9 β fragments, as shown in Table 15 in the 650-5650 m/z window would correspond to a 34.04% sequence coverage for the α chain and a 88.36% sequence coverage for the β chain. The low sequence coverage for the α chain is due to the small number of fragments produced when subjected to endoproteinase Glu C digest, where out of the five possible fragments, two fragments are too small (αG2 and αG3) and one is too large (αG4) to be detected within the 650-5650 m/z window. For the β chain, there are more detectable fragments within the 650-5650 m/z window. For a 3 min on carrier digest, a sequence coverage of 21.28% for the α chain and 48.23% for the β chain was achieved, as shown in FIG. 10. The detected fragments along with their respective theoretical masses, resolved masses, respective ppms, sequence coverage by each detected fragment are listed in Table 15.

TABLE 15
Mass accuracy of fragments derived from an on carrier 3 min
endoproteinase Glu C digestion of whole unpurified blood.

| Mass | Mass | | | | | | |
|---|---|---|---|---|---|---|---|
| [m + H]⁺ | [m + H]⁺ | | | | Number | Sequence | Missed |
| Theoretical | Received | Δppm | Fragment | Position | of AA | Coverage | Cleavage |
| 2306.2251 | 2306.2427 | −7.6 | α G1 | 1-23 | 23 | 16.31% | 0 |
| 2726.3848 | 2726.3876 | −1.0 | α G1-2 | 1-27 | 27 | 19.15% | 1 |
| 3039.5533 | 3039.5823 | −9.5 | α G1-3 | 1-30 | 30 | 21.28% | 2 |
| 824.4148 | 824.3976 | −20.9 | β G1-2 | 1-7 | 7 | 4.96% | 1 |
| 2422.2612 | 2422.2842 | 9.5 | β G1-3 | 1-22 | 22 | 15.60% | 2 |
| 2764.4151 | 2764.3241 | −32.9 | β G1-4 | 1-26 | 26 | 18.44% | 3 |
| 4840.546 | 4840.8385 | 60.4 | β G1-5 | 1-43 | 43 | 30.50% | 4 |
| 1745.9068 | 1745.9124 | 3.2 | βG2-3 | 7-22 | 22 | 15.60% | 1 |
| 1616.8642 | 1616.7608 | −64.0 | β G3 | 8-22 | 15 | 10.64% | 0 |
| 2437.3026 | 2437.3137 | 4.6 | β G4-5 | 23-43 | 21 | 14.89% | 1 |
| 2095.1487 | 2095.2029 | 25.9 | β G5 | 27-43 | 17 | 12.06% | 0 |
| 2680.4357 | 2680.3922 | −16.2 | βG9 | 122-146 | 25 | 17.73% | 0 |

This table corresponds to the MALDI-ToF mass spectrum in FIG. 10.

The number of fragments detected within 10 ppm was 3 for each chain. The β chain of human Hb possess two consecutive endoproteinase Glu C specific amino acids, Glu⁶ and Glu⁷, and it was observed that endoproteinase Glu C hydrolysed the chain at both amino acid residues producing the fragments, in increasing m/z, βG1-2 (m/z value of 824.3936, pos 1-7), βG3 (m/z value of 1616.7608, position 8-22), βG2-3 (m/z value of 1745.9068, position 7-22), confirming the phenomena, as shown in FIG. 11A, B, C. It is assumed that once the enzyme has cut C-terminal after Glu⁷, it is unable to cut again after Glu⁶, because it is classified as an endoproteinase. As a result of incomplete digestion, smaller fragments are detected as combined larger peptide fragments, such as αG2 and αG3 (m/z 439.1823 and 332.1816 respectively) fragments are detected as αG1-2 (m/z 2726.3876), and αG1-3 (m/z 3039.5823).

### Example 5

### Application of the Methods for Identification of Known Hb Variants. Methods of Determining the Identity of a Polypeptide

Since the best results for the on carrier tryptic digestion of Hb A in whole human blood were obtained with the ionic surfactant RapiGest™ SF at 37° C. at 3 min digest time, this procedure was applied to the blood samples with known Hb variants at a 1:100 dilution along with two samples with unknown Hb variants, listed in Table 16. When subjected to a digest, the Hb chain containing a substitution of an amino acid, due to the presence of a mutation in the corresponding gene, results in a mass shift of a specific fragment, the appearance of new signature peptide/s as a result of addition of a cleavage site or disappearance of fragment/s followed by appearance of new fragment/s as a result of deletion of a cleavage site. An elongation or a deletion of a chain segment would also be reflected by a mass shift of the corresponding fragment/s.

TABLE 16
List of variants identified using the newly established MALDI-ToF
MS method for screening haemoglobin variants with their amino
acid substitution, resulting mass shift and m/z values.

| Variant | Chain | Pos. | Substitution | [m + H]⁺ | Shift |
|---|---|---|---|---|---|
| Hb J Toronto | α | A5 | Ala > Asp | 15171.38 | 44.10 |
| Hb Setif | α | A94 | Asp > Tyr | 15175.46 | 48.09 |
| Mutant New 0302 | β | B37 | Trp > Cys | 15785.15 | −83.07 |
| Hb Marseille | β | B2 | +Met, His > Pro | 158959.40 | 91.17 |
| HB S | β | B6 | Glu > Val | 15838.25 | −29.98 |
| Hb J-Kaohsiung | β | B59 | Lys > Thr | 15841.16 | −27.07 |
| HB E | β | B26 | Glu > Lys | 15867.89 | −0.94 |
| HB C | β | B6 | Glu > Lys | 15867.89 | −0.94 |
| Mutant New 08 | β | B54 | Val > Leu | 15882.26 | 14.03 |
| Hb TyGard | β | B124 | Pro > Gln | 15899.24 | 31.01 |
| Hb J-Bangkok | β | B56 | Gly > Asp | 15926.23 | 58.00 |

The monoisotopic masses of the peptide fragments were calculated with the program Peptide Mass at the ExPASy website http://kr.expasy.org/cgi-bin/peptide-mass.pl. The information on individual mutants was taken from the Globin Server at http://globin.cse.psu.edu.

### Establishment of a Library of Identifiable Hb Variants

In the following section the Hb variants are grouped according to the impact of the enzyme on the number of fragments and the use of enzymes.
A. Additional cleavage site: A1. Trypsin, A2. Endoproteinase Glu C
B. Mass shift only, number of cleavage site maintained: B1. Trypsin, B2. Endoproteinase Glu C
C. Loss of a cleavage site: C1. Trypsin, C2. Endoproteinase Glu C
D. Elongation of globin chains.

### A. Hb Variants with Amino Acid Substitution Resulting in an Additional Cleavage Site

Substitution of a particular amino acid with another amino acid which constitutes a cleavage site to a certain enzyme results in producing additional proteolytic fragments. The substitution of an amino acid with “Lys”, a specific amino acid for trypsin, in any position would result in two new tryptic fragments for a complete cleavage, resulting in an additional fragment. If incomplete cleavages occur, then several additional fragments may occur. These additional fragments can be used as signature peptide to identify Hb variants.

### A1. On Carrier Digestion with Trypsin

### Hb E Variant

In the following, the newly developed method including a time course investigation is applied to Hb E. The Hb E (α₂ββ_{E}) is characterised by a Glu²⁶ to Lys²⁶ mutation whereby the resulting β_{E} chain differs from the normal β chain by a molecular mass of 0.94 Dalton. In Hb E, in one of the β chains, the normal βT3 fragment VNVDEVGGEALGR is converted to β_{E}T3 and β_{E}T4 by the introduction of an additional cleavage site VNVDEVGGK/ALGR, yielding two unique fragments with expected monoisotopic masses of [M+H]⁺ 916.4734 and 416.2616. As a consequence, all subsequent fragments of the β_{E}-chain have to be renumbered, although they are identical, i.e. βT10=β_{E}T11.

### Linear Mode Screening

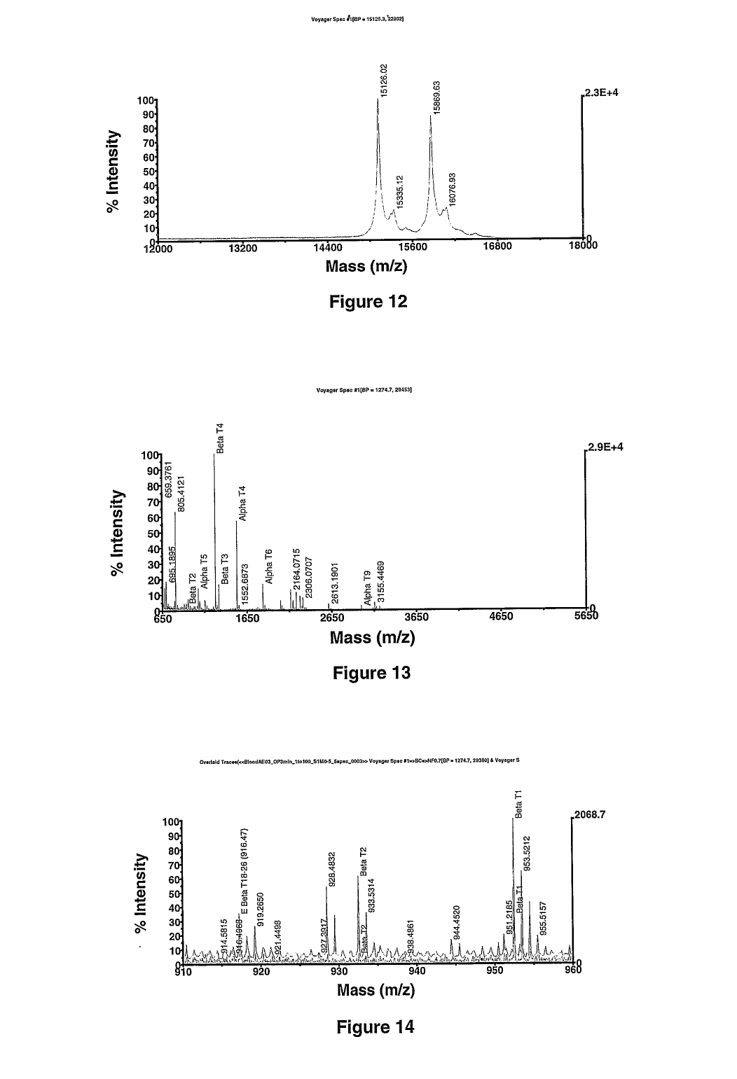

The mass spectrum of human blood containing a Hb E (α₂ββ_{E}) variant shows the double charged (received m/z values [M+2H]⁺⁺/2: 7557.4 and 7927.8) and single charged (received m/z values [M+H]⁺: 15125.1 and 15869.4) Hb E α chain and β chains, respectively, whereby the βchain and β_{E} chain, could not be resolved, as shown in FIG. 12. The associated error was −2.3 Dalton for the α chain and 1.18 to 2.12 Dalton for the β chains. It is evident that from the spectra obtained in the linear mode the Hb E variant cannot be identified.

### Identification of the Hb E Signature Peptides by on Carrier Trypsin Digestion

In this experiment, a time course on carrier tryptic digestion was performed. The on carrier tryptic digestion of Hb E in whole human blood obtained with the ionic surfactant RapiGest™ SF at 37° C. with a 3 min digest time resulted in a spectrum with 100% sequence coverage for the α chain and β chain, respectively, shown in FIG. 13. The Hb E signature peptide β_{E}T3 VNVDEVGGK was detected with a mass accuracy of 2.1 ppm (expected 916.4734, received, 916.4715), and thus the Hb E variant was unambiguously identified, as shown in FIG. 14. A minor peak of a second Hb E signature peptide β_{E}T2-3 (SAVTALWGKVNVDEVGGK) with a lower mass accuracy of 163.9 ppm (expected 1829.9755, received, 1829.6755) was also detected. Since the tetrapeptide β_{E}T4 was neither detected as a single fragment nor as part of a peptide with missed cleavage sites, the resulting sequence coverage of the resulting β_{E} chain was 97.16%. In the digest of normal Hb A, the fragments αT4, αT5, αT6, αT9, and βT1, βT3, βT4, βT5, βT13, βT occur as single fragments. In the Hb E digest however, αT4, αT5, αT6, αT9, αT12, αT13, and βT1, βT3, βT4, βT5/β_{E}T6, βT12/β_{E}T13, βT13/β_{E}T14, βT14/β_{E}T15 occur as single fragments. Surprisingly, and in contrast to the digest of normal Hb A, in Hb E the fragments αT12, αT13, and βT12/β_{E}T13, that are believed to precipitate during the tryptic digest, were detected as single fragments. The time course experiment showed, that the Hb E-variant was cleaved at all time points of digestion much more effectively then the normal Hb A, which may be related to the reported instability of Hb E. Interestingly, two γ-chain fragments were detected, namely γT2-3 with a m/z value of 2274.0368 (expected 2274.1724) and γT10-12 (a fragment generated by cleavage after Lys⁷⁶ which is an additional cleavage site of the γchain in respect to the βchain) with a m/z value of 3250.3203 (expected 3249.5996) with a mass accuracy of 58.8 and 221.8 ppm, respectively. The detection of γchain fragments is in agreement with reported elevated Hb F levels for individuals having the Hb E variant.

Overall the results demonstrate the general applicability of the newly developed method. In the following further experiments, no time course experiment was done; instead the optimised conditions for a 3 min on carrier digest in the presence of the novel surfactant at 37° C. were applied.

### Hb C Variant

The Hb variant Hb C (α₂ββ_{C}) is characterised by a Glu⁶ to Lys⁶ substitution, whereby the resulting β_{C} chain differs from the normal βchain by a molecular mass of −0.94 Dalton. In Hb C, in one of the β chains, the normal βT1 fragment VHLTPEEK is converted to β_{C}T1 and β_{C}T2 by the introduction of an additional cleavage site VHLTPK/EK, yielding two unique fragments with expected monoisotopic masses of [M+H]⁺ 694.4246 and 276.1554. As a consequence, all subsequent fragments of the β_{C} chain have to be renumbered, although they are identical, ie. βT2=β_{C}T3.

### Linear Mode Screening

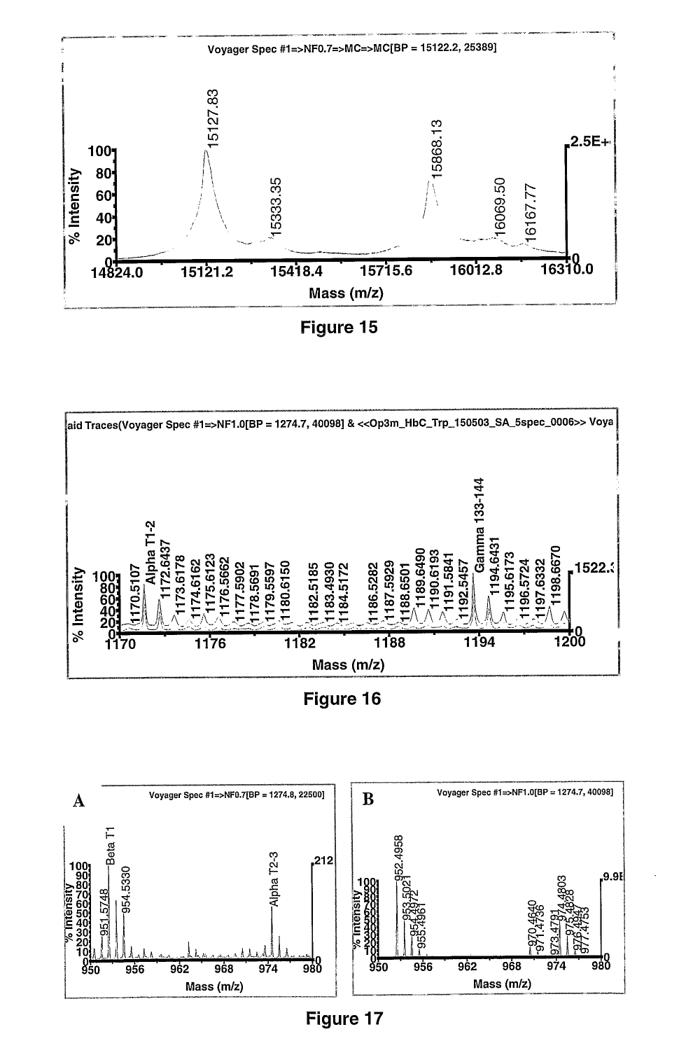

The mass spectrum of human blood containing an Hb C (α₂ββ_{C}) variant shows the double charged (received m/z values [M+2H]⁺⁺/2: 7627.23 and 7994.77) and single charged (received m/z values [M+H]⁺: 15127.83 and 15868.13) Hb C α chain, β and β_{C} chains, respectively, whereby the β chain and β_{C} chain, could not be resolved, as shown in FIG. 15, further confirming that a mass shift up to 5 Da cannot be resolved with current the MALDI-ToF instrument using the linear mode. The associated error was 0.46 Da for the α chain and −0.1 to −0.24 Da for the ββ_{C} chains.

### Identification of the Signature Peptides by on Carrier Trypsin Digestion

The Hb C signature peptides β_{C}T1 and β_{C}T2 could not be detected with the current settings, as these smaller fragments were lost in the matrix background. However, a signature peptide clearly specific for the Hb C variant, β_{C}T2-3, EKSAVTALWGK, was detected with a mass accuracy of 7.14 ppm (expected m/z value 1189.6575, received, 1189.6490) and thus the Hb C variant was identified, as shown in FIG. 16. The overlaid traces in FIG. 16 show the absence of any peak where signature peptide β_{C} T2-3 appeared.

A minor peak of a second Hb C signature peptide β_{C}T1-2, VHLTPK/EK, was detected with a lower mass accuracy of −13.24 ppm (expected m/z value 951.5622, received 951.5748), which indicates that a 0.935 Da mass shift to the left can be detected with the settings used in this invention using the reflector mode, as shown in FIGS. 17 A and B. Here a spectrum with a monoisotopic mass [M+H]⁺ 952.4958 in panel B is obtained from blood containing normal Hb A, whereas panel A shows a spectrum with a mass shift to the left with a low abundance monoisotopic peak [M+H]⁺ 951.5748 obtained from blood containing the Hb C variant.

The presence of the signature peptides for Hb C confirms its presence, but at the same time the presence of the βT1 [M+H]⁺ 952.4958 fragment is of high significance. The presence of this peak confirms the heterozygous state for Hb C and the presence of the normal β chain, whereby the absence of which would imply a homozygous state for the variant. The additional cleavage site may account for the low abundance of the β_{C}T1-2 peptide in the digestion products. Since in a heterozygous state for haemoglobin C, only 30-40% of the total haemoglobin content is haemoglobin C, the decreased signal intensity of β_{C}T1-2 (resolved m/z 951.5748) when compared with its normal counterpart can be explained. The low ion abundance for β_{C}T1-2 may also be the reason for its low mass accuracy.

### B. Hb Variants with an Amino Acid Substitution Resulting in the Same Number of Cleavage Sites and a Mass Shift for the Signature Peptides

### B1. On Carrier Digestion with Trypsin

### Hb S

The haemoglobin variant Hb S (α₂ ββ_{S}) is characterised by a Glu¹²⁴ to Val¹²⁴ (E to V) mutation in the β chain, whereby the resulting β_{S} chain differs from the normal α chain by a molecular mass of −29.98 Da.

### Linear Mode Screening

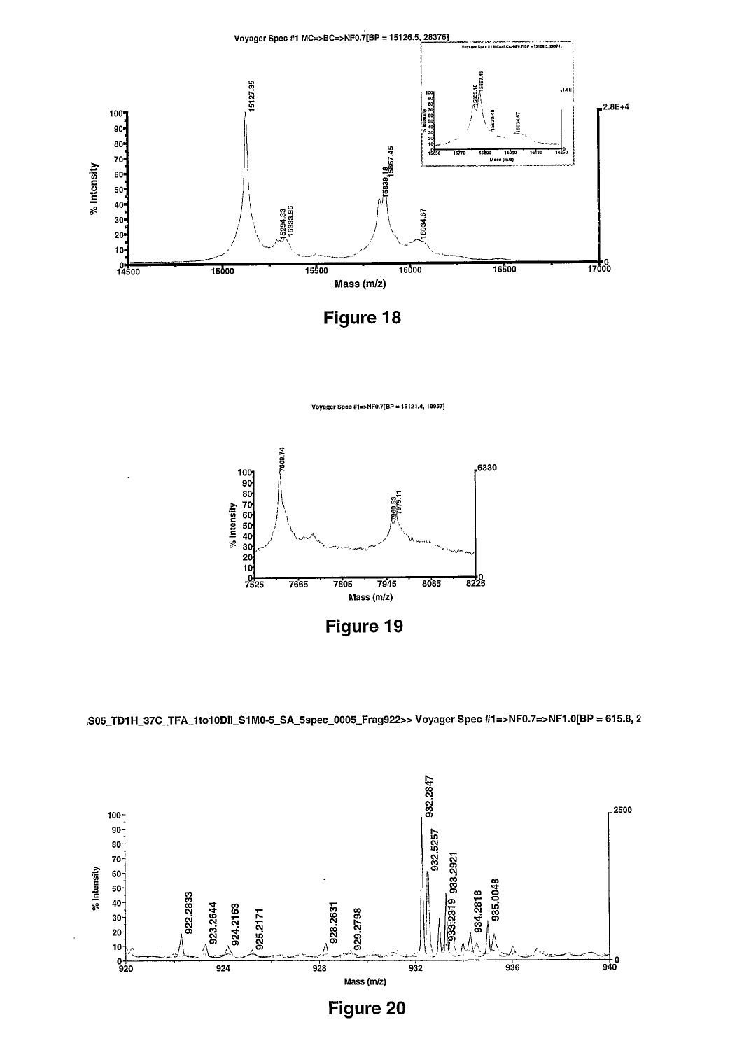

The mass spectrum of human blood containing a Hb S (α₂ββ_{S}) variant shows the single charged [M+H]⁺ average m/z value of 15127.35 (expected m/z value 15127.37) representative for the α chain and the [M+H]⁺ average m/z values 15867.45 (expected m/z value 15868.23) and 15839.18 (expected m/z value 15838.25) for the β and β_{S} chain, respectively, whereby the β chain and β_{S} chain, had a mass difference of −30.3 Da (expected mass shift −29.98 Da), as shown in FIG. 18. The split in the β peak is representative of a heterozygous state, where as in a homozygous state for Hb S only one peak (β_{S}) with a mass shift −31.01 Da from the β peak would have been resolved. The associated error was −0.02 Da for the α chain, −0.78 Da for the β chain and 0.93 Da for the β_{S} chain. The mass spectrum of human blood containing the Hb S (α₂ββ_{S}) variant shows also the double charged [M+2H]⁺⁺/2: value 76974.22 and a split in the second peak, yielding m/z values of 7960.53/7975.11, as shown in FIG. 19.

### Identification of the Hb S Signature Peptides by on Carrier Trypsin Digestion

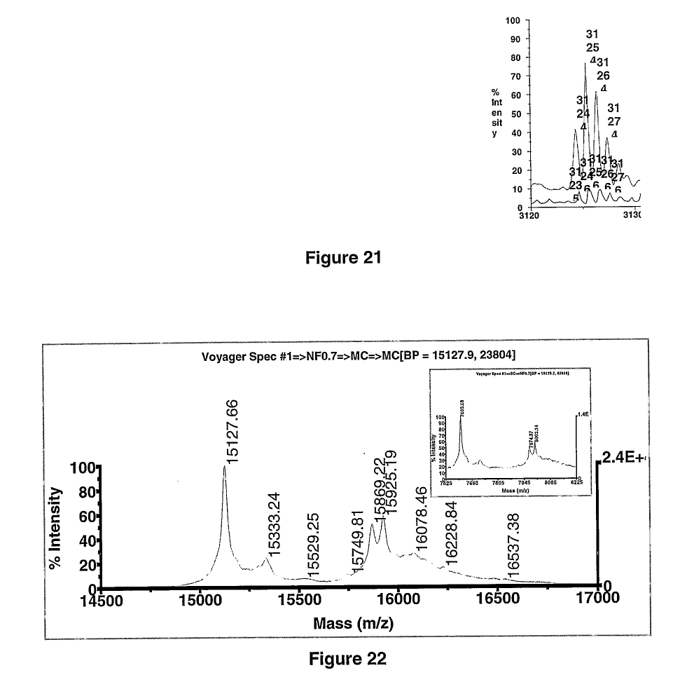

In Hb S heterozygotes, due to substitution of an amino acid in one of the β chains, the normal βT1 fragment, [M+H]⁺ with a monoisotopic mass 952.5098, VHLTPEEK is converted to smaller tryptic fragments β_{S}T1, VHLTPEVK, with an expected monoisotopic masses of [M+H]⁺ 922.5356, the βT1-2 fragment, VHLTPEEKSAVTALWGK, [M+H]⁺ 1866.0119, is converted to β_{S}T1-2, VHLTPEVKSAVTALWGK, with an expected monoisotopic masses of [M+H]⁺ 1836.0377, and the βT1-3 fragment, VHLTPEEKSAVTALWGKVNVDEVGGEALGR, [M+H]⁺ 3161.6589, is converted to β_{S}T1-3, VHLTPEVKSAVTALWGKVNVDEVGGEALGR, with an expected monoisotopic mass of [M+H]⁺ 3131.6847. The on carrier tryptic digestion of Hb S heterozygote (α₂ββ_{S}) in whole human blood obtained with the ionic surfactant RapiGest™ SF at 37° C. and 3 min digest time, yielded two signature peptides, the β_{S}T1, as shown in FIG. 20, and the β_{S}T1-3, as shown in FIG. 21, with a mass accuracy of 273.4 and −12.1 ppm respectively, as shown in Table 17. Interestingly, β_{S}T1-2 was not detected. The appearance of an additional β chain which had a peak with −30.3 Da smaller mass than the normal β peak in the linear mode and emergence of the two signature peptides unique for the Hb S variant detected in reflector mode unambiguously identified the sample as one from an individual carrying a Hb S. Here, the presence of two signature peptides results in a high confidence identification. Alongside, the presence of normal βT1, βT1-3 confirms the heterozygous state. Furthermore, the presence of the normal βT2-3 tryptic fragment aids in localizing the substitution to be in βT1.

TABLE 17
Identified signature peptides for Hb S, with mass accuracy.

| | | | Missed | Theoretical | Received | |
|---|---|---|---|---|---|---|
| Fragment | Position | Sequence | Cleavage | Mass | Mass | ppm |
| β_{S}T1 | 1-8 | VHLTPEVK | 0 | 922.5356 | 922.2833 | 273.4 |
| β_{S}T1-3 | 1-30 | VHLTPEVKSAVT | 2 | 3131.6847 | 3131.7227 | −12.1 |
| | | ALWGKVNVDEV | | | | |
| | | GGEALGR | | | | |

### Hb J Bangkok

The Hb variant Hb J-Bangkok, also known as Hb J-Korat, Hb J-Manado or Hb J-Meinung, (α₂ ββ_{J-Bangkok}) is characterised by a Gly⁵⁶ to Asp⁵⁶ (G to D) mutation in the β chain, whereby the resulting PJ-Bangkok chain differs from the normal β chain by a molecular mass of 58 Da.

### Linear Mode Screening

The associated error was −0.29 Da for the [symbol] chain, −0.99 Da for the β chain and 1.04 Da for the β_{J-Bangkok} chain. The split in the β chain confirms the heterozygous state. The mass spectrum of human blood containing an Hb J-Bangkok (α₂ββ_{J-Bangkok}) variant showed also the double charged globin chains m/z value [M+2H]⁺⁺/2: 7605.08 and a split in second peak, 7974.37/8003.14), also shown in FIG. 22 (inset).

### Identification of the Hb J Bangkok Signature Peptide by on Carrier Trypsin Digestion

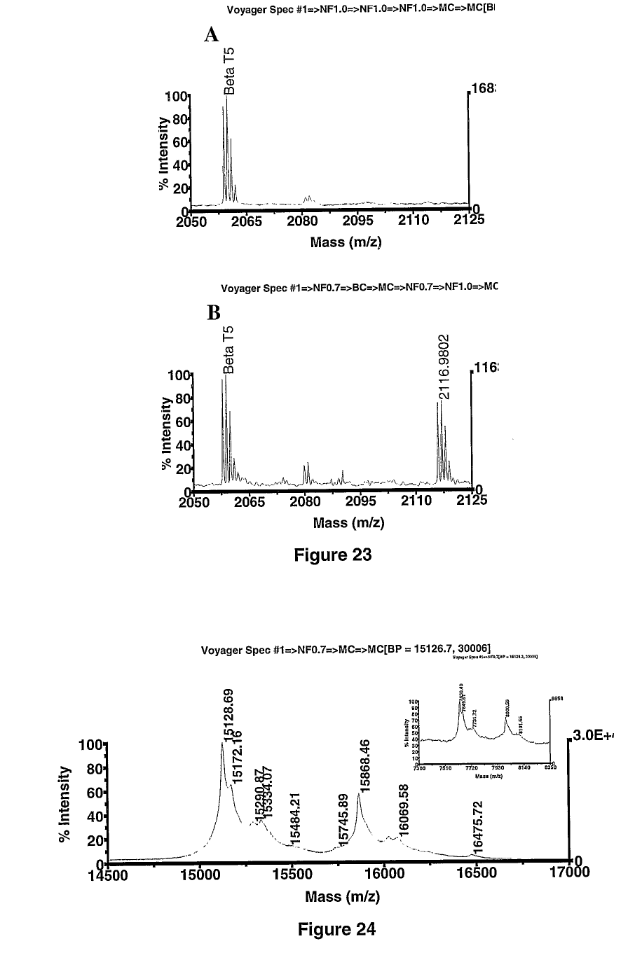

In Hb J-Bangkok heterozygotes, due to substitution of an amino acid in one of theβ chains, the normal βT5 fragment with the monoisotopic mass [M+H]⁺ of 2058.9477, FFESFGDLSTPDAVMGNPK is converted to the β_{J-Bangkok} T5 fragment, FFESFGDLSTPDAVMDNPK, with an expected monoisotopic masses of [M+H]⁺ 2116.9531. The on carrier tryptic digestion of haemoglobin J-Bangkok (α₂ββ_{J-Bangkok}) in whole human blood obtained with the ionic surfactant RapiGest™ SF at 37° C. and a 3 min digest time produced the signature peptide, β_{J-Bangkok} T5, with a mass accuracy of −3.12 ppm, where as the its counterpart, normal βT5 was detected with a mass accuracy of 9.23 ppm, as shown in FIG. 23 B, with a m/z window of 2050-2125. The received and expected masses for the signature peptide along with their mass accuracy are listed in Table 18. The spectrum in FIG. 23 A is obtained from a normal blood sample containing Hb A (α₂β₂) whereby no peak other than the normal βT5 is detected in the same m/z window of 2050-2125. The appearance of an additional β peak, 55.97 Da larger than the normal p peak, in the linear mode and the emergence of the signature peptide unique for the Hb J-Bangkok variant detected in the reflector mode unambiguously identified the sample to come from an individual carrying a Hb J-Bangkok variant. The Hb J-Bangkok carrier state was confirmed by the presence of the normal counterpart of the signature peptide. The absence of the normal βT4-5 and βT5-6 fragments were interesting since they were usually resolved on a 3 min digests of normal Hb A at 37° C. with the presence of the novel surfactant, although the peaks had relatively weak signals. The absence of these peaks, and the corresponding peaks with the substitutions, may be explained by the low abundance of these peptides resulting from the low amount of normal and mutated globin chains in a carrier state.

TABLE 18
Identified signature peptide for Hb J-Bangkok carrier state, with mass accuracy.

| | | | Missed | Theoretical | Received | |
|---|---|---|---|---|---|---|
| Fragment | Position | Sequence | Cleavage | Mass | Mass | ppm |
| β_{J-Bangkok} T5 | 1-8 | FFESFGDLSTP | 0 | 2116.9531 | 2116.9597 | −3.12 |
| | | DAVMGNPK | | | | |
| βT5 | 1-30 | FFESFGDLSTP | 0 | 2058.9477 | 2058.9581 | 9.23 |
| | | DAVMDNPK | | | | |

### Hb Setif

The haemoglobin variant Hb Setif is an α chain variant (αα_{Setif}β₂). It is characterised by an Asp⁹⁴ to Val⁹⁴ (N to Y) substitution in the α chain, whereby the resulting α_{Setif} chain differs from the normal a chain by a molecular mass of +48.09 Da.

### Linear Mode Screening

The mass spectrum of human blood containing a Hb Setif (α_{Setif}β₂) variant shows the single charged [M+H]⁺ average m/z value of 15128.69 (expected m/z value 15127.37 Da) for the α chain, a [M+H]⁺ average m/z value of 15172.56 Da (expected m/z value 15175.46) for α_{Setif} and a [M+H]⁺ average m/z value of 15868.46 (expected m/z value 15868.23) for the β chain. The α chain and the α_{Setif} chain, had a mass difference of 44.79 (expected mass shift 48.09) as shown in FIG. 24. The associated errors were 1.32 Da for the α chain, 3.3 Da for the α_{Setif} chain and 0.23 Da for the β chain. The mass spectrum of human blood containing the Hb Setif (α₁α_{Setif}β₂) variant also showed the double charged [M+2H]⁺⁺/2 value of 7630.40 and 7649.61 resulting from two α chains and a m/z value of 8000.54 for the β chain, as shown in FIG. 24 (inset).

### Identification of the Hb Setif Signature Peptide by on Carrier Trypsin Digestion

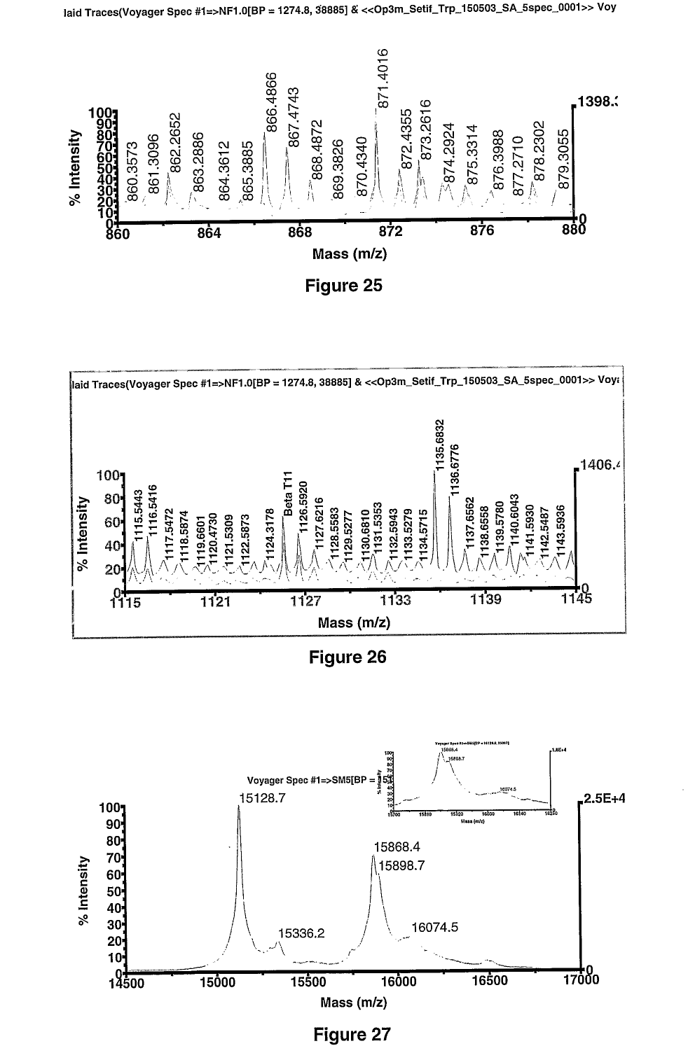

In Hb Setif heterozygotes, due to substitution of an amino acid in one of the [symbol] chains, the normal αT11 fragment with a monoisotopic [M+H]⁺ mass of 818.4406, VDPVNFK is converted to α_{Setif}T11, VYPVNFK, with an expected monoisotopic mass of [M+H]⁺ 866.4770 Da, and a αT10-11 fragment, LRVDPVNFK, [M+H]⁺ 1087.6258 Da, is converted to α_{Setif} T10-11, LRVYPVNFK, with an expected monoisotopic mass of [M+H]⁺ 1135.6622 Da. The on carrier tryptic digestion of the Hb α variant, Hb Setif (αα_{Setif} β₂), in whole human blood obtained with the ionic surfactant RapiGest™ SF at 37° C. and a 3 min digest time yielded two signature peptides, α_{Setif} T11, as shown in FIG. 25, and α_{Setif}T10-11, as shown in FIG. 26, with a mass accuracy of 35.9 and −46.1 ppm respectively, as listed in Table 19. The appearance of two α peaks representing two α chains with a mass difference of 48.09 Da in the linear MALDI-ToF MS mode confirms the heterozygous state for an α chain variant and the detection of the two signature peptides unique for the Hb Setif variant in reflector mode unambiguously identified the sample to come from an individual carrying Hb Setif chain, i.e., a Hb Setif carrier.

TABLE 19
Identified signature peptides for Hb Setif, with mass accuracy.

| | | | Missed | Theoretical | Received | |
|---|---|---|---|---|---|---|
| Fragment | Position | Sequence | Cleavage | Mass | Mass | ppm |
| α_{Setif} T11 | 93-99 | VYPVNFK | 0 | 866.4770 | 866.4459 | 35.9 |
| α_{Setif} T10-11 | 91-99 | LRVYPVNFK | 1 | 1135.6622 | 1135.7146 | −46.1 |

### B 2. On Carrier Digestion with Endoproteinase Glu C

### Haemoglobin TyGard

The Hb Ty Gard (α₂β_{TyGard}) is a β chain variant and is characterised by a Pro¹²⁴ to Gly¹²⁴ (P to G) mutation in the β chain, whereby the resulting β_{TyGard} chain differs from the normal β chain by an average molecular mass of +31.01 Da.

### Linear Mode Screening

The mass spectrum of human blood containing a TyGard (α₂ββ_{TyGard}) variant shows the single charged [M+H]⁺ average m/z value of 15128.7 Da (expected m/z value 15127.37 Da) representative for the α chain and a [M+H]⁺ average m/z value of 15868.40 Da (expected m/z value 15868.23 Da) and 15898.70 Da (expected m/z value 15899.24 Da) for β and β_{TyGard} chains, respectively, whereby the βchain and β_{TyGard} chain, had a mass difference of 30.3 Da (expected mass shift 31.01 Da) as shown in FIG. 27. The associated error was 1.33 Dalton for the α chain, 0.17 Da for the α chain and 0.54 Da for the PTyGard chain. The mass spectrum of human blood containing an TyGard (α₂ββ_{TyGard}) variant shows the double charged m/z value [M+2H]⁺⁺/2: 7554.22 Da and a split in the second peak with m/z values 7927.8 Da and 7938.96 Da.

### Identification the Hb Tygard Signature Peptide by on Carrier Glu C Digestion

In Hb TyGard heterozygotes, due to substitution of an amino acid in one of the β chain, the normal βG9 fragment with a monoisotopic [M+H]⁺ m/z value of 2680.4357 Da, is converted to β_{TyGard}G9 with an expected monoisotopic mass [M+H]⁺ of 2711.4457 Da, as shown in Table 20.

TABLE 20
Signature peptide for Hb TyGard identification.

| | | | Missed | Theoretical | Received | |
|---|---|---|---|---|---|---|
| Fragment | Position | Sequence | Cleavage | Mass | Mass | ppm |
| β_{TyGard}G9 | 122-146 | FTGPVQAAYQK | 0 | 2711.4457 | 2711.445 | −0.37 |
| | | VVAGVANAL | | | | |
| | | AHKYH | | | | |
| βG9 | 122-146 | FTPPVQAAYQK | 0 | 2680.4357 | 2680.436 | −0.22 |
| | | VVAGVANAL | | | | |
| | | AHKYH | | | | |

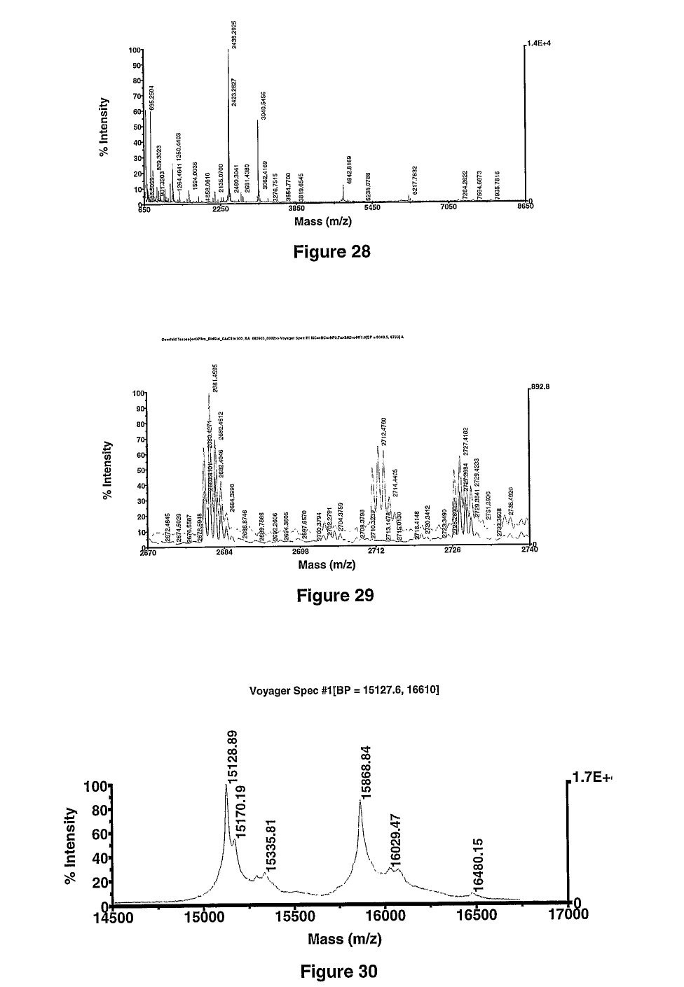

The on carrier endoproteinase Glu C digestion of haemoglobin TyGard (α₂ββ_{TyGard}) in whole human blood obtained with the ionic surfactant RapiGest™ SF at 37° C. and a 3 min digest time resulted in a spectrum with similar sequence coverage for the α chain and β chain, respectively, achieved for normal blood showing similar fragmentation pattern when digested with endoproteinase GluC, as shown in FIG. 28. In the spectrum, four β chain fragments were detected in the 10 ppm window, with increasing masses, βG4, βG3-4, βG9, βG5 (data not shown). The signature peptide βG9 FTGPVQAAYQKVVAGVANALAHKYH was detected with a mass accuracy of −0.3 ppm, (expected 2711.4457, received, 2711.445), as depicted in Table 20 and shown in FIG. 29. The appearance of an additional β peak confirmed a heterozygous state for a β Hb variant and the appearance of the signature peptide for the variant Hb TyGard (α₂ββ_{TyGard}) identified the carrier status for Hb TyGard of the sample with confidence.

### Hb J-Toronto

The Hb variant Hb J Toronto (αα_{J-Toronto} β₂) is characterised by an Ala⁵ to Asp⁵ (A to N) substitution in the α chain, whereby the resulting α_{J-Toronto} chain differs from the normal α-chain by a molecular mass of +44 Da.

### Linear Mode Screening

The mass spectrum of human blood containing a Hb J-Toronto (αα_{J-Toronto} β₂) variant shows the single charged [M+H]⁺ average m/z value of 15128.89 Da (expected m/z value 15127.37 Da) representative for the [symbol] chain, a [M+H]⁺ average m/z value of 15170.19 Da (expected m/z value 15171.38 Da) for α_{J-Toronto} and a [M+H]⁺ average m/z value of 15868.84 Da (expected m/z value 15868.23 Da) for the βchain. The αchain and α_{J-Toronto} chain had a mass difference of 43.0 Da (expected mass shift 44.1 Da) as shown in FIG. 30. The associated error was 1.52 Da for the chain, 1.13 Da for the α_{J-Toronto} chain and 0.61 Da for the β chain. The mass spectrum of human blood containing an Hb J-Toronto (α₁α_{J-Toronto}β₂) variant shows the double charged [M+2H]⁺⁺/2: value of 7619.43 and 7631.10 (split in the α peak) and a m/z value of 7991.73.

### Identification of the Hb J-Toronto Signature Peptide by an on Carrier Endo-Proteinase GluC Digest.

In Hb J Toronto heterozygotes the substitution of Ala⁵ to Asp⁵ (A to N) in one of th [symbol] [symbol] chain yields three signature peptides identifiable by a 3 min on carrier endoproteinase Glu C digest with RapiGest™ SF at 37° C. The first signature peptide is α_{J-Toronto}G1, VLSPNDKTNVKAAWGKVGAHAGE, with an expected mono-isotopic mass of [M+H]⁺ 2350.2149 Da, where as its counterpart, the normal αG1 fragment has a monoisotopic [M+H]⁺ m/z value of 2306.3896 Da (VLSPADKTNVKAAWGKVGAHAGE). The second signature peptide is a result of substitution in the αG1-2 fragment with 1 missed cleavage, VLSPADKTNVKAAWGKVGAHAGEYGAE, having a monoisotopic [M+H]⁺ m/z value of 2726.3896 Da. The α_{J-Toronto}G1-2 fragment, the second signature peptide, VLSPNDKTNVKAAWGKVGAHAGEYGAE, has an expected monoisotopic mass of [M+H]⁺ 2770.3794. The third signature peptide is converted from the normal αG1-2 fragment, VLSPADKTNVKAAWGKVGAHAGEYGAEALE, with a monoisotopic [M+H]⁺ m/z value of 3039.5533 Da. The α_{J-Toronto}G1-3 signature peptide fragment has an expected monoisotopic mass of [M+H]⁺ 3083.5432 Da (VLSPNDKTNVKAAWGKVGAHAGEYGAEALE).

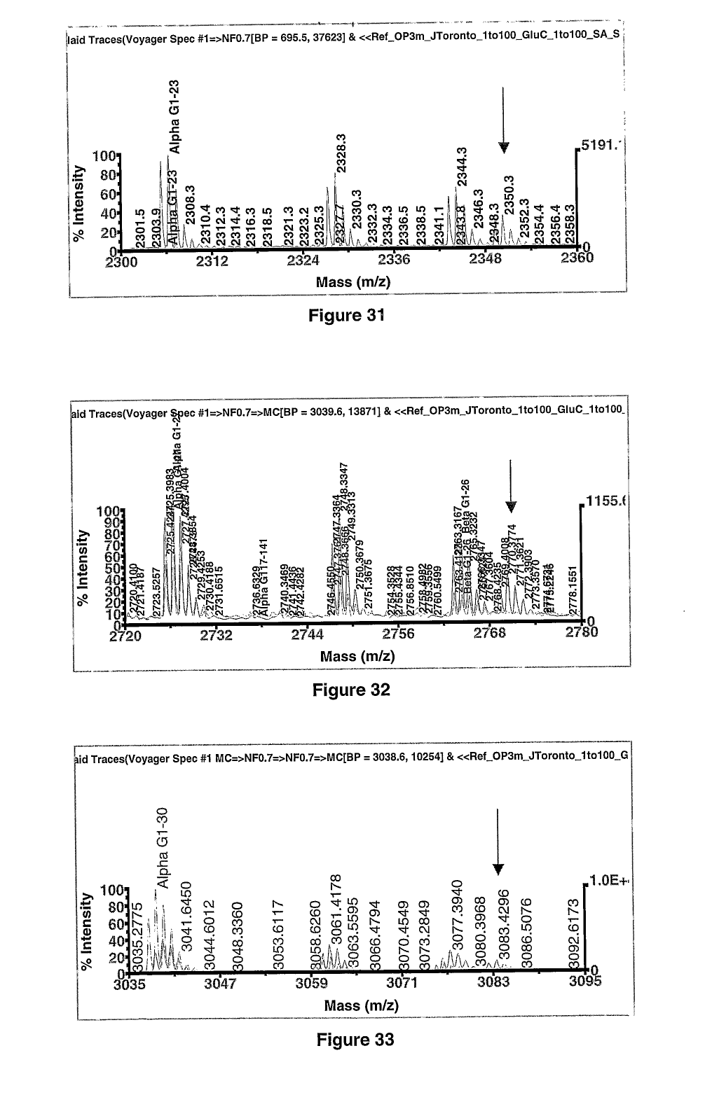

The 3 min on carrier tryptic digestion of the Hb α variant, J-Toronto (αα_{J-Toronto} β₂), in whole human blood obtained with the ionic surfactant RapiGest™ SF at 37° C. resulted in three signature peptides, the α_{J-Toronto}G1, as shown in FIG. 31, the α_{J-Toronto}G1-2, as shown in FIG. 32, and finally the α_{J-Toronto}G1-3, as shown in FIG. 33, which were resolved with a mass accuracy of −13.3, −42.3 and −37.5 ppm respectively, as listed in Table 21.

TABLE 21
Identified signature peptides for Hb J-Toronto with mass accuracy.

| | | Missed | Theoretical | Received | |
|---|---|---|---|---|---|
| Fragment | Sequence | Cleavage | Mass | Mass | ppm |
| αG1 | VLSPADKTNVKAA | 0 | 2306.3896 | 2306.2731 | 50.5 |
| | WGKVGAH AGE | | | | |
| α_{J-Toronto}G1 | VLSPNDKTNVKAA | 0 | 2350.2149 | 2350.2461 | −13.3 |
| | WGKVGAH AGE | | | | |
| αG1-2 | VLSPADKTNVKAA | 1 | 2726.3896 | 2726.4895 | −36.6 |
| | WGKVGAH | | | | |
| | AGEYGAE | | | | |
| α_{J-Toronto}G1-2 | VLSPNDKTNVKAA | 1 | 2770.3794 | 2770.4967 | −42.3 |
| | WGKVGAH | | | | |
| | AGEYGAE | | | | |
| αG1-3 | VLSPADKTNVKAA | 2 | 3039.5533 | 3039.7387 | −61.0 |
| | WGKVGAH | | | | |
| | AGEYGAE ALE | | | | |
| α_{J-Toronto}G1-3 | VLSPNDKTNVKAA | 2 | 3083.5432 | 3083.6587 | −37.5 |
| | WGKVGAH | | | | |
| | AGEYGAE ALE | | | | |

The normal counterparts of these fragments, the αG1, the αG1-2 and the αG1-3, were also detected with mass accuracy of 50.5, −36.6 and −61.0 ppm, respectively. The appearance of an additional peak besides the normal α peak with a mass shift of +43.0 Da in the linear mode and the detection of the three signature peptides unique for the Hb J Toronto variant in reflector mode unambiguously identified the sample to come from an individual carrying Hb J-Toronto. The two peaks in the liner mode and the detection of the αG1, the αG1-2 and the αG1-3 fragments confirm the Hb J-Toronto carrier state.

### C. Variants with Amino Acid Substitution Resulting in Loss of a Cleavage Site and a Measurable Mass Shift

### C₁. On Carrier Digestion with Trypsin

### Haemoglobin J-Kaohsiung

The Hb variant Hb J-Kaohsiung, (α₂ββ_{J-Kaohsiung}) is characterised by a Lys⁵⁹ to Thr⁵⁹ (K to T) change in the β chain, whereby the resulting β_{J-Kaohsiung} chain differs from the normal βchain by a molecular mass of −27.07 Da. The substitution of Lys, an amino acid which is a specific cleavage site for trypsin, to Thr results in the loss of a cleavage site. As a consequence, βT5 and βT6 merge to form β_{J-Kaohsiung}T5, with a mass shift of −27.07 Daltons, and subsequent fragments of the β_{J-Kaohsiung} have to be renumbered, although they are identical, i.e. βT7=β_{J-Kaohsiung}T6.

### Linear Mode Screening

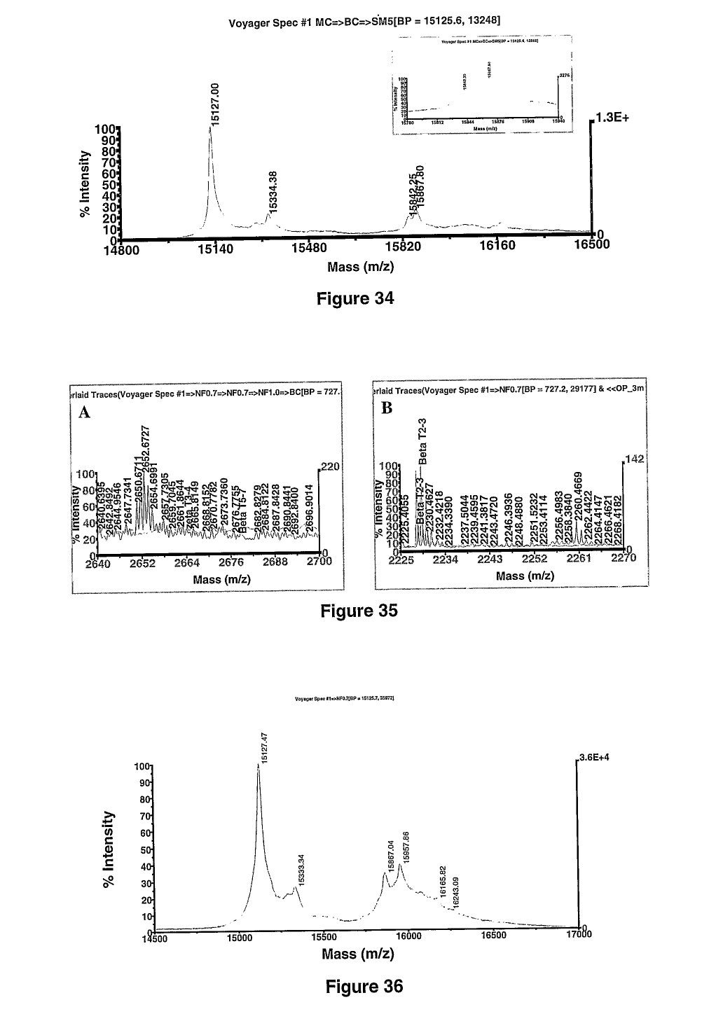

The mass spectrum of human blood containing a J-Kaohsiung variant, (α₂ββ_{J-Kaohsiung}) shows the single charged [M+H]⁺ average m/z value of 15127.00 Da (expected m/z value 15127.37 Da) representative of the α chain and a [M+H]⁺ average m/z value of 15867.80 Da (expected m/z value 15868.23 Da) and 15842.85 Da (expected m/z value 15841.16 Da) for the β and the β_{J-Kaohsiung} chains, respectively, whereby the βchain and β_{J-Kaohsiung} chain, had a mass difference of −25.55 Da (expected mass shift −27.07 Da) as shown in FIG. 34. The associated error was −0.37 Da for the α chain, −0.43 Da for the β chain and −1.09 Da for the β_{J-Kaohsiung} chain. The mass spectrum of human blood containing a J-Kaohsiung (α₂ββ_{J-Kaohsiung}) variant also shows the double charged [M+2H]⁺⁺/2 m/z value of 7554.22 Da and split of the second peak with m/z values of 7927.8 Da and 7938.96 Da.

### Identification of the Signature Peptides by on Carrier Trypsin Digestion

In Hb J-Kaohsiung heterozygotes, due to substitution of Lys to Thr in one of the β-chains resulting in a deletion of a cleavage site, the normal βT5-6 fragment with a monoisotopic [M+H]⁺ m/z value of 2486.1110 Da, is converted to β_{J-Kaohsiung}T5 with an expected monoisotopic mass of [M+H]⁺ 2259.0638 Da, the normal βT5-7 fragment, [M+H]⁺ 2679.3235 Da, is converted to β_{J-Kaohsiung}T5-6 with an expected monoisotopic mass of [M+H]⁺ 2652.2762 Da, as shown in Table 22.

The on carrier trypsin digestion of Hb J-Kaohsiung (α₂ββ_{J-Kaohsiung}) in whole human blood obtained with the ionic surfactant RapiGest™ SF at 37° C. and a 3 min digest time allowed the detection of the signature peptides, β_{J-kaohsiung}T5, FFESFGDLSTPDAVMGNPTVK, with a monoisotopic mass of 2259.4464 Da (expected [M+H]⁺ m/z value 2259.0638 Da) and a mass accuracy of −169.3 ppm and β_{J-Kaohsiung}T5-6, FFESFGDLSTPDAVMGNPTVKAHGK, with a monoisotopic mass of 2652.6727 Da (expected [M+H]⁺ m/z 2652.2762 Da) and a mass accuracy of −49.4 ppm, as shown in FIGS. 35 A and B.

TABLE 22
Identified signature peptide fragments for Hb J-Kaohsiung with mass accuracy.

| | | | Missed | Theoretical | Received | |
|---|---|---|---|---|---|---|
| Fragment | Position | Sequence | Cleavage | Mass | Mass | ppm |
| βT5-6 | 41-61 | FFESFGDLSTPDA | 1 | 2486.1110 | Weak signal | X |
| | | VMGNPKVK | | | | |
| β_{J-Kaohsiung}T5 | 41-61 | FFESFGDLSTPDA | 0 | 2259.0638 | 2259.4464 | −169.3 |
| | | VMsGNPTVK | | | | |
| βT5-7 | 41-65 | FFESFGDLSTPDA | 2 | 2679.3235 | Not detected | X |
| | | VMGNPKVK | | | | |
| | | AHGK | | | | |
| β_{J-Kaohsiung}T5-6 | 41-65 | FFESFGDLSTPDA | 1 | 2652.2762 | 2652.6727 | −149.4 |
| | | VMGNPTVK | | | | |
| | | AHGK | | | | |

From the theoretical point of view, the β_{J-Kaohsiung}T5 fragment with a monoisotopic [M+H]⁺ m/z of 2259.4464 Da, has a identification conflict with the γT62-82 fragment with a monoisotopic [M+H]⁺ m/z value of 2259.2812 Da. However the mass value received can be seen as to belong to β_{J-Kaohsiung} since the low abundance of Hb F (α₂γ₂) in adult blood can be assumed.

The appearance of β_{J-Kaohsiung}T5-6 (AA 41-65) was an interesting observation, as the normal βT5-7 (AA 41-65) fragment was not detected in this invention, as documented, in FIG. 8 and the normal βT5-6 (AA 41-61) was only captured as a weak signal, whereby the signal for β_{J-Kaohsiung}T5 (AA 41-65) was more intense. It may be due to fact that the deletion of a cleavage site, and the Thr substitution for Lys, results in a peptide with altered properties favouring MALDI-ToF MS detection.

Although the signature peptides for J-Kaohsiung (α₂ββ_{J-Kaohsiung}) were detected with lower mass accuracy, believed to be result of the low abundance of the peptides, appearance of two signature peptides unambiguously identified the Hb variant J-Kaohsiung (α₂β₂β_{J-Kaohsiung}). The appearance of two β peaks in the linear mode confirms the heterozygous state for a Hb variant J-Kaohsiung.

### D. Variants with Elongated Globin Chains

### Haemoglobin Long Island

The Hb variant Hb Long Island, also known as Hb Marseille, (α_{2ββLongIsland}) is characterised by an extension of the N-terminus by a Met (M) residue, and a His² (H) to Pro² (H to P) substitution in the β chain, whereby the resulting β_{LongIsland} chain differs from the normal βchain by a molecular mass of 91.17 Da (Met addition would result in a 131.04 Da shift, the H is >Pro would result in a −40.2 Da shift, finally resulting in a net change of 131.04-40.02=91.17 Da).

### Linear Mode Screening

The mass spectrum of human blood containing a Long Island (α₂ββ_{LongIsland}) variant shows the single charged [M+H]⁺ average m/z value of 15127.47 Da (expected m/z value 15127.37 Da) representative for the [symbol] chain and a [M+H]⁺ average m/z value of 15867.04 Da (expected m/z value 15868.23 Da) and 15957.86 Da (expected m/z value 15959.40 Da) for β and β_{LondIsland} chains, respectively, whereby the βchain and β_{LondIsland} chain, had a mass difference of 90.9 Da (expected mass shift 91.17 Da) as shown in FIG. 36. The associated error was 0.1 Da for the [symbol] chain, −1.19 Da for the βchain and 1.54 Da for the β_{LongIsland} chain. The mass spectrum of human blood containing a Hb LongIsland (α₂ββ_{LondIsland}) variant shows the double charged [M+2H]⁺⁺/2 m/z values of 7554.22 Da and a split of the second peak with m/z values of 7927.8 Da and 7938.96 Da.

### Identification the Signature Peptide by on Carrier Glu C Digestion

In Hb LongIsland heterozygotes, one of the β chains, the normal βG1-3 fragment, [M+H]+ 2422.264, is converted to β_{LongIsland}G1-3 with an expected monoisotopic mass of [M+H]⁺ 2513.10189 Da, as shown in Table 23.

TABLE 23
Identified signature peptide for Hb Long Island with mass accuracy.

| | | | Missed | Theoretical | Received | |
|---|---|---|---|---|---|---|
| Fragment | Position | Sequence | Cleavage | Mass | Mass | ppm |
| βG1-3 | 1-22 | VHLTPEEKSAV | 1 | 2422.2614 | 2422.19 | 29.4 |
| | | TALWGKVNVDE | | | | |
| β_{LongIsland}G1-3 | 1-22 | MVPLTPEEKSA | 0 | 2513.1019 | 2513.14 | −15.9 |
| | | VTALWGKVNVD | | | | |

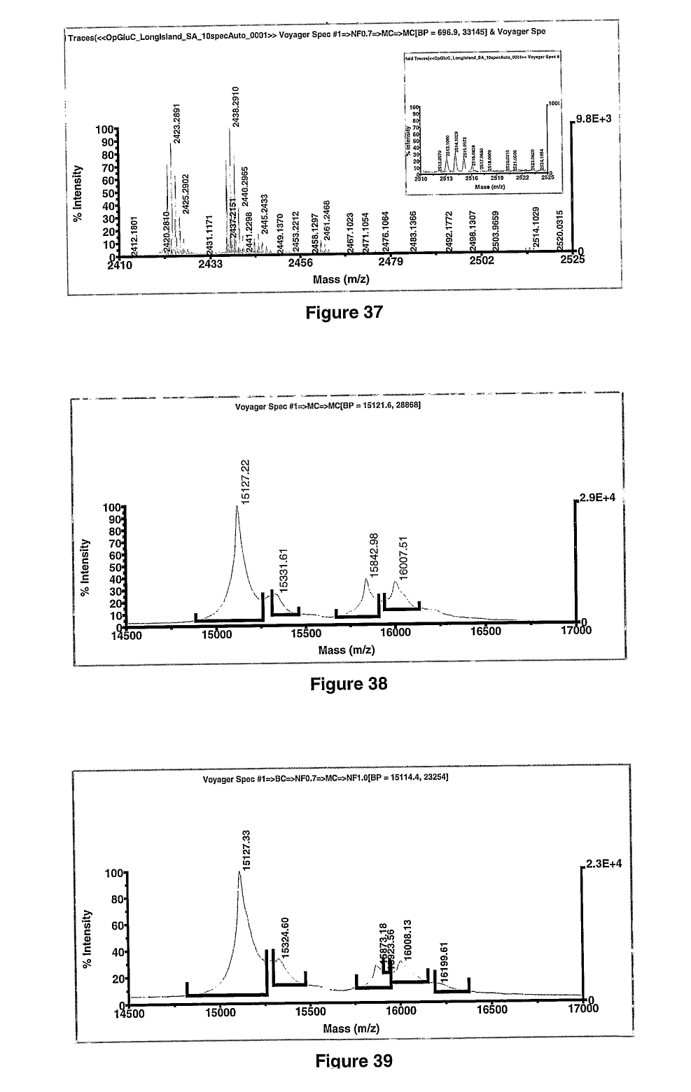

The on carrier endoproteinase Glu C digestion of haemoglobin Long Island (α₂ββ_{LongIsland}) in whole human blood obtained with the ionic surfactant RapiGest™ SF at 37° C. with a 3 min digest time resulted in a spectrum with similar sequence coverage for the α chain and β chain, respectively, achieved for normal blood showing similar fragmentation pattern when digested with endoproteinase Glu C with an extra peak, as shown in FIG. 37. The signature peptide *βG1-3, MVPLTPEEKSAVTAL-WGKVNVD, was detected with a mass accuracy of −15.9 ppm (expected m/z value 2513.1019 Da, received m/z value of 2515.1400 Da), and thus the Hb Long Island (α₂ββ_{LongIsland}) variant was unambiguously identified, as shown in FIG. 37 (inset). The lower mass accuracy is believed to be a result of a low abundance of the peptide. Other possible signature peptides such as β_{LongIsland}G1-2 and β_{LongIsland}G1-4 were not seen although weak signals for βG1-2, and βG1-4 were detected, which also believed to be a result of the low abundance of the β_{LongIsland}G1-2, and β_{LongIsland}G1-4 fragments. The appearance of a second β peak in the linear mode confirms the heterozygous state for the variant.

### Example 6

### The quantitative aspects of MALDI-ToF MS

The quantitative aspects of MALDI-ToF MS have been reported in the literature. In this invention quantitative aspects of MALDI-TOF MS in respect to haemoglobinopathies have been explored. Variation of different Hb levels is characteristic of many β Hb variants. The following table (Table 24) represents the level of different Hbs characteristic for some β thalassaemias and their interactions with Hb variants (modified).

TABLE 24
Hb levels characteristic for different thalassaemia and Hb variants.

| Thalassaemia | Homozygous | Heterozygous |
|---|---|---|
| β⁰ | Hb F 90% | Hb A₂ 3.5-7% |
| β⁺ | Hb F 70-95% | Hb A₂ 3.5-7% |
| β⁺Thal. intermedia | Hb F 20-40% | Hb A₂ 3.5-7% |
| Hb S | Hb S 30-40% | |
| Hb S β⁰ | Hb S 85%, Hb F 10% | |
| Hb S β⁺ | Hb S 65-80%, Hb F 5% | |
| Hb E β⁰ | Hb E 60-70%, Hb F 30-40% | |

### Sickle Thalassaemia

Four patient samples from known sickle thalassaemia and Hb S heterozygote with known HPLC results were investigated using the MALDI-ToF MS linear mode. The peak area represents the ion species abundance which reflect the amount of the proteins. The peak area was calculated using the Data Explorer Software and the sum of the peak areas representing [symbol] , βₛ, δ and γ chains were added (100%) proportions were calculated accordingly. For each sample, 5 consecutive spectra were obtained whereby each spectrum was an accumulation of 5 spectra each obtained using 100 laser shots. The different chain amounts measured by MALDI-ToF MS showed remarkable similarity with HPLC results with some variations, as shown in Table 25. Although Hb F, Hb S and Hb were measurable, it was observed that with the current MALDI-ToF MS instrument the low abundance Hb proportions cannot be measured. The Hb A₂ levels and Hb F levels obtained from samples from the sickle thalassaemia patient are listed in Table 24. The spectrum shown in FIG. 38 represents the sample form the sickle thalassaemia patient.

TABLE 25
Different Hb proportions measured by MALDI-ToF MS
using peak areas, and HPLC results.

| Sample | Hb Chain | MALDI | HPLC |
|---|---|---|---|
| Sample 1 | Hb F(γ) | 41.59% | 45.90% |
| | Hb S | 58.41% | 44.30% |
| | A₂ (δ) | | 3.60% |
| Sample 2 | Hb F(γ) | 49.58% | |
| | Hb S (βₛ) | 50.42% | |
| Sample AS1 | Hb A (β) | 45.28% | |
| | Hb S (βₛ) | 54.72% | |
| Sample AS2 | Hb A (β) | 58.77% | 50.50% |
| | Hb S | 41.23% | 39.70% |
| | Hb (γ) | | 0.50% |

Number of spectra per sample: 5.

### Thalassaemia Intermedia

A sample from known thalassaemia intermedia patient with a HPLC quantification report of the Hb proportions were investigated, as shown in Table 26. It was observed that in this particular instance Hb A₂ was measurable but not with confidence. The β and the γ chains show good correlation with the HPLC report. The corresponding spectrum is depicted in FIG. 39.

TABLE 26
Proportion of different globin chains measured with MALDI-ToF in the
linear mode using the peak area and the corresponding HPLC report.

| Globin | | | |
|---|---|---|---|
| Chains | Peak Area | Peak Area % | HPLC report |
| β | 1547815.462 | 38.61% | 30.1% |
| δ | 27405.5271 | 0.68% | 4.8% |
| γ | 2433156.299 | 60.70% | 58.0% |

### Post-Translational Modification of Hb

Almost all proteins contain transient or permanent post-translationally modified amino acids such as glycosylated, acetylated, methylated or hydroxylated amino acids. The most common post-translational modification for haemoglobin is glycated Hb whereby the N-terminal valine of the β chain is irreversibly glycated known as the minor Hb A_{1C} fraction. But ESI MS and MALDI-TOF MS studies revealed that glycation occurs in both α and β chains and other glycated proteolytic fragments have been investigated in some reports. The glycation sites of Hb reported by Shapiro et al. show various Val and Lys positions of both the chains as major glycation sites. These post-translational modifications may hinder proteolytic activity.

In this invention, the glycation adducts of patients with different Hb A_{1C} level determined by HPLC method were investigated using the MALDI-ToF MS linear mode. Additionally investigations were carried out to examine if any glycated proteolytic fragments were detectable using on carrier 3 min endoproteinase Glu C digestion in the presence of RapiGest™ at 37° C.

### Glycated α and β Chains

Three whole blood samples having Hb A_{1C} levels of 10.0%, 8.8% and 5.4% and diluted 1:100 with ammonium bicarbonate buffer were screened using the MALDI-TOF MS linear mode. The globin chains and the adducts were resolved with a grid voltage and delay time set to 90% and 350 ns respectively. The resolved m/z values were within 1 standard deviation from the expected masses (listed in Table II), as shown in Table 27.

TABLE 27
The m/z values of intact globin chains, glycated
globin chains and SA adducts.

| | Intact Globin | | |
|---|---|---|---|
| | Chain (SD) | Glycated globin chain | SA adducts (SD) |
| Chain | m/z value | (SD) m/z values | m/z values |
| α | 15128.19(1.4) | 161.5(1.9) | 206.8(0.5) |
| β | 15868.91(1.5) | 162.8(1.8) | 207.2(1.1) |

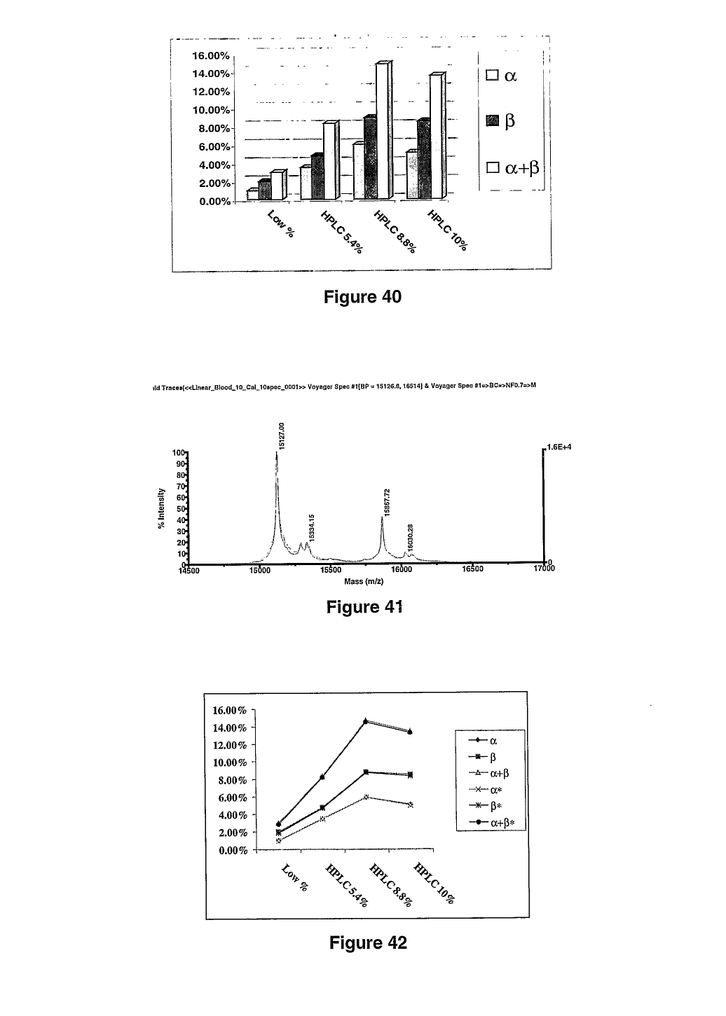

The peak areas relate to the abundance of an ionic species in MALDI-ToF MS, as such the peak areas for each resolved m/z values were calculated using the Data Explorer software. The percentages for glycated and not glycated globin chains were calculated for individual globin chains and in total by summing all areas of all detected species (100%) and individual species as proportion of the total area, as shown in Table 28. It is evident from FIGS. 40, 41 and 42 and Table 28 that both the chains are glycated, although the β chain shows a higher glycation rate for all the samples. The mean of the ratio for α and β glycation for the glycated samples were 0.63 (SD0.03). This shows that the higher glycation level for the β chains were independent of the glycation level of samples in agreement with reports in the literature. It is also observed that the β glycation percentage measured by the MALDI-ToF MS linear mode yields results closer to the HPLC result, where as the total glycation measured by MALDI-ToF MS yields result that are higher. Yet, the results show that MALDI-TOF MS results are more or less consistent with the reported glycation levels.

TABLE 28
MALDI-ToF MS measurement of glycation in intact globin chains.

| | | | | |
|---|---|---|---|---|
| Hb Chain | Low | 5.4 | 8.8 | 10 |

Glycation % A (Excluding the SA adduct area).

| | | | | |
|---|---|---|---|---|
| α | 1.00% | 3.47% | 5.91% | 5.01% |
| β | 1.96% | 4.76% | 8.80% | 8.47% |
| Total | 2.96% | 8.24% | 14.71% | 13.48% |

Glycation % B (including the SA adduct area).

| | | | | |
|---|---|---|---|---|
| α | 0.99% | 3.45% | 5.88% | 4.99% |
| β | 1.87% | 4.70% | 8.69% | 8.33% |
| Total | 2.86% | 8.16% | 14.57% | 13.32% |

SA %

| | | | | |
|---|---|---|---|---|
| α | 1.44% | 0.59% | 0.55% | 0.49% |
| β | 1.74% | 1.38% | 1.42% | 1.75% |
| Total | 3.18% | 1.97% | 1.97% | 2.23% |

Number of obtained spectra per sample: 10; SD of area measurements: 0.01%.

The overlaid MALDI-TOF MS spectra obtained in the linear mode from 5.4% glycated and 10.0% glycated samples show that the peak for the β glycation adduct has a comparatively higher peak height than the α glycation adduct, as shown in FIG. 41.

For this invention, the percentages for glycated and not glycated globin chains were calculated for either excluding (Glycation % A) or including the SA adduct area (Glycation % B) to observe the effect of such calculations, interestingly which show that no significant deviation of calculated total glycation percentage occurs if the SA adduct area is left out of the calculation, as shown in FIG. 40. Another interesting finding was that the MALDI-TOF MS measured result (14.71%) for the sample with the HPLC report of 8.8% glycation was higher than the one for the sample with the HPLC report of 10.0% glycation (13.48%), whereby both α and β chain for the 8.8% (HPLC) (MALDI-TOF MS 14.71%) showed a higher glycation amount.

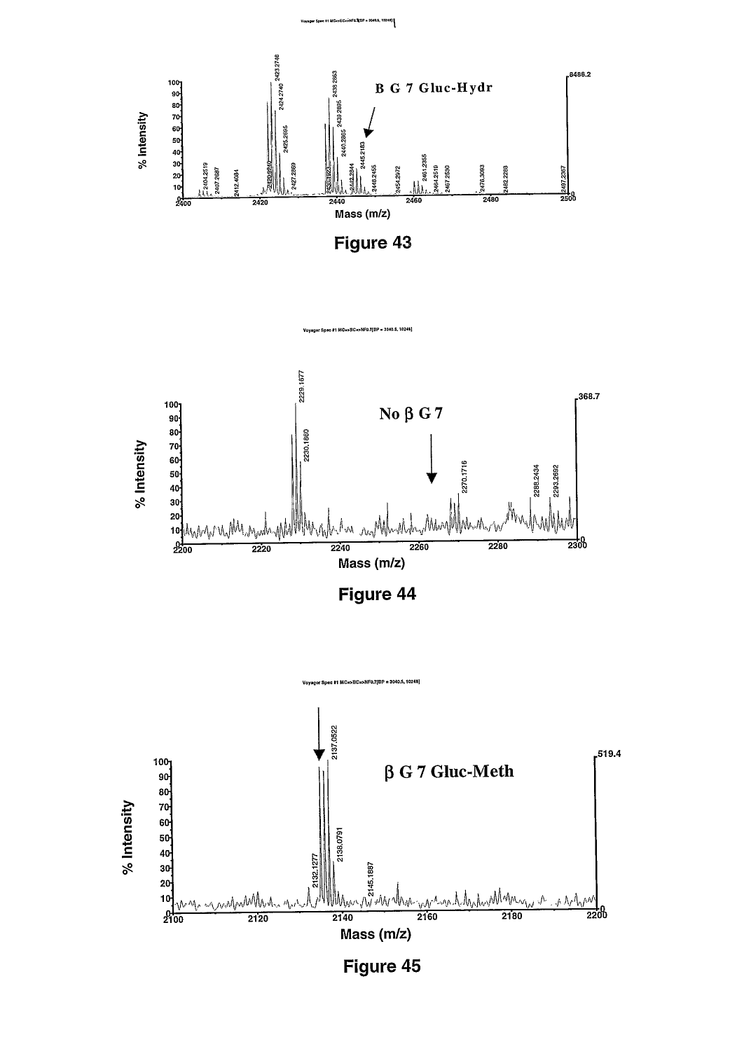

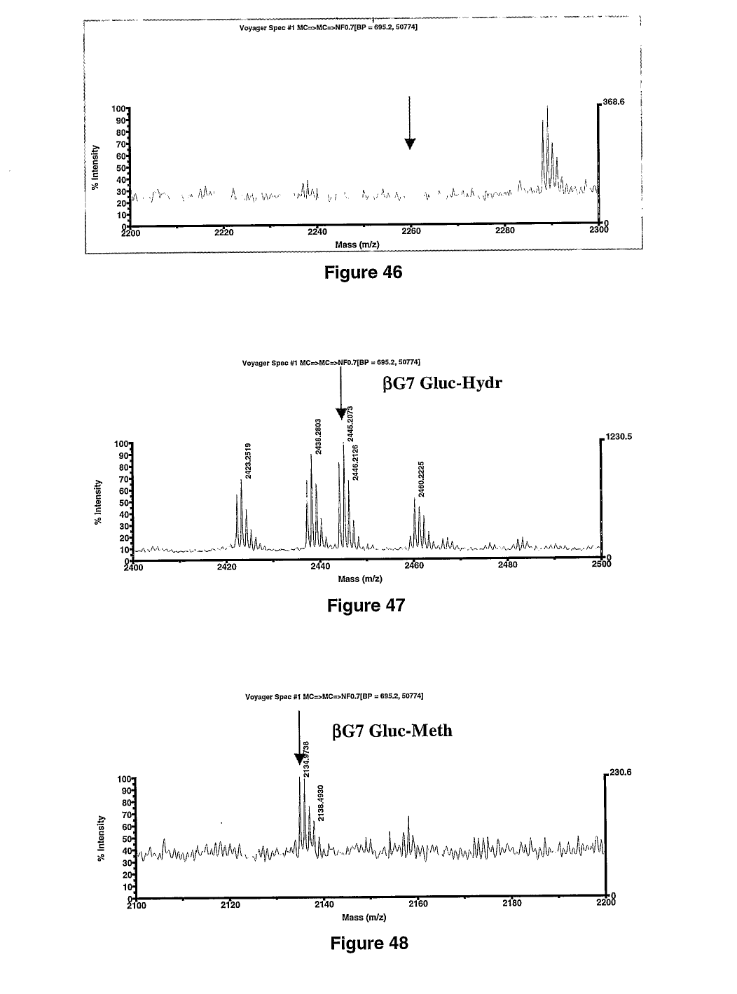

Determination of the presence of glycated peptide peaks and its identification is important for the interpretation of spectra obtained from an on carrier proteolytic digest. To investigate if any glycated globin peaks can be identified, two on Glu C digests were carried out as initial experiments on unpurified EDTA treated blood samples with normal and high glycated Hb proportions (10.0%). The resulting spectra were compared. The same glycated peaks were identified in both the samples but with clearly different signal intensity using the ExPASy FindMod tool, as shown in FIGS. 43 and 45 for normal blood sample, and in FIGS. 46 and 48 for the blood sample with a high glycation level.

In here, two fragments, the glycated and hydroxylated fragment βG8 and the methylated βG3-4 were detected. It was also interesting to observe that the normal counterpart of the peptide fragment, βG8, was not detectable with present experimental conditions, neither for the blood sample with normal nor for the sample with a high Hb glycation level, as shown in FIGS. 44 and 47.

While investigating the peaks it was observed that only one of the glycated peaks, βG8 Gluc-Hydr, whereby the glucose molecule is attached to the β Lys¹²⁰, has shown a visible difference in the peak obtained from normal and the peak obtained from sample with high glycation. To investigate this finding further, the peak heights, relative intensities, and peak areas of the monoisotopic and most abundant peaks of βG8 Gluc-Hydr were compared with the respective values from the adjacent peak βG4-5. The ratios between the peaks are listed in Table 29 showing an increased ratio for the glycated sample for all three parameters. The appearance of the glycated peptides needs further investigation to confirm its origin, sequence and other relevant mass spectrometric properties.

TABLE 29
Extend of glycation of proteolytic fragment βG8 by ratio
of peak heights, relative intensities and peak areas of the
βG8 in relation to the βG4-5 peaks.

| | | | Relative | |
|---|---|---|---|---|
| | | Height | Intensity | Area |
| Normal blood sample | Monoisotopic peak | 0.81 | 0.81 | 1.10 |
| Normal blood sample | Most abundant peak | 0.90 | 0.90 | 0.96 |
| Blood sample with | Monoisotopic peak | 3.62 | 3.62 | 3.89 |
| high glycation level | | | | |
| Blood sample with | Most abundant peak | 3.55 | 3.55 | 4.02 |
| high glycation level | | | | |

The MALDI-TOF mass spectra shown in FIGS. 46, 47 and 48, were obtained in the linear mode from an on carrier 3 min digest in the presence of the novel surfactant at 37° C. from unpurified blood sample, diluted 1:100, containing a glycation level of 10.0% reported by HPLC.

### Variation of Trypsin Concentration for on Carrier Digestion

The effect of trypsin concentration variation for the on carrier digestion of whole human blood in presence of the novel surfactant RapiGest™ was investigated. Although the general effect of shortened digest time on the tryptic fragmentation pattern has been reported, a systematic investigation on trypsin concentration on the fragmentation pattern of the Hb α and β chain is not reported in the literature. In this experiment, the aim was to document the proteolytic fragmentation pattern, optimise on carrier trypsin concentration in relation to the sequence coverage, establish method robustness and check compatibility with automated data analysis.

For this experiment, trypsin stock solution with a trypsin concentration of 1.3 mg/ml (54.5 μM) equalling 5.45 pM/μl was diluted 1:10, 1:20, 1:40, 1:80 and 1:100 fold with 50 mM ammonium bicarbonate buffer, 2 mM CaCl₂, pH 8.3. For an on carrier digestion 2 μl of each dilution of trypsin and 2 μl of stock solution without dilution was spotted for each digest on the sample plate and let air dried at room temperature. Three different samples, two blood samples collected from two individuals with normal blood and one blood sample with Hb S, were investigated. For each sample, 3 independent 3 min digests were carried out with the novel method devised in this invention, using the ionic surfactant, on carrier at 37° C. For each digest spot, 10 MALDI-TOF mass spectra were obtained, whereby each spectrum was an accumulation of 5 spectra, each obtained from 100 laser shots. The data were analysed using the Protein Prospector software. It was observed, which adds to the confidence of automated detection of globin chains, that the overall MOWSE score for the detected peptides were high. (MOWSE scores >75 are considered to be significant for protein identification). Although there was a variation in the number of α and βfragments identified within the 10 ppm window, it was constantly higher in trypsin stock solution diluted 1:20 and higher, for normal blood and blood with Hb S variant, as depicted in Table 31.

TABLE 30
The α and the β fragments identified within a 10 ppm mass
accuracy window in different trypsin dilution for blood sample.

| | Globin | | |
|---|---|---|---|
| Trypsin dilution | chain | Peaks identified | Mowse Score |
| 1 to 10 | α | 5 | 5.48E+02 |
| | β | 5 | 4.38E+02 |
| 1 to 20 | α | 4 | 1.16E+02 |
| | β | 7 | 4.38E+02 |
| 1 to 40 | α | 5 | 8.43E+02 |
| | β | 5 | 1.74E+02 |
| 1 to 80 | α | 4 | 5.48E+02 |
| | β | 8 | 1.10E+03 |
| 1 to 100 | α | 6 | 1.05E+02 |
| | β | 7 | 1.10E+03 |

Number of spectra analyzed: 10 for each dilution.

TABLE 31
The α and the β fragments identified within the 10 ppm
mass accuracy window in different trypsin dilution for Hb S.

| Trypsin | Globin | Fragments | |
|---|---|---|---|
| dilution | chain | identified(SD) | MOWSE score |
| 1 to 10 | α | 5(3) | 2.16E+03 |
| | β | 5(3) | 1.80E+02 |
| 1 to 20 | α | 6(1) | 1.74E+03 |
| | β | 4(1) | 3.94E+03 |
| 1 to 40 | α | 5(1) | 6.34E+02 |
| | β | 4(1) | 1.39E+02 |
| 1 to 80 | α | 7(2) | 1.34E+03 |
| | β | 4(1) | 6.15E+01 |

Number of spectra analyzed: 10 for each dilution.

Interestingly, MALDI-ToF mass spectra obtained for the three samples demonstrate similar fragmentation pattern for each dilution, but they differ in different dilutions. It was observed that a concentration above 1:20 fold stock solution result in loss of bigger tryptic fragments necessary for higher sequence coverage for both chains, which results from a decrease in partial digestion products. To demonstrate this highly significant observation, the clinically important tryptic fragment of βT1 (m/z 952.5098) and partially digested fragments βT2-3(m/z 2228.1669), β1-3 (m/z 3161.6589) were investigated. βT1, sequence positions 1-8, contains Glu at position 6, substitution of which result in Hb S. In the newly established method, in an on carrier digest on normal blood, the βT1, and partially digested fragments βT2-3, βT1-3 fragments are resolved at all time points between 50 s and 3 min.

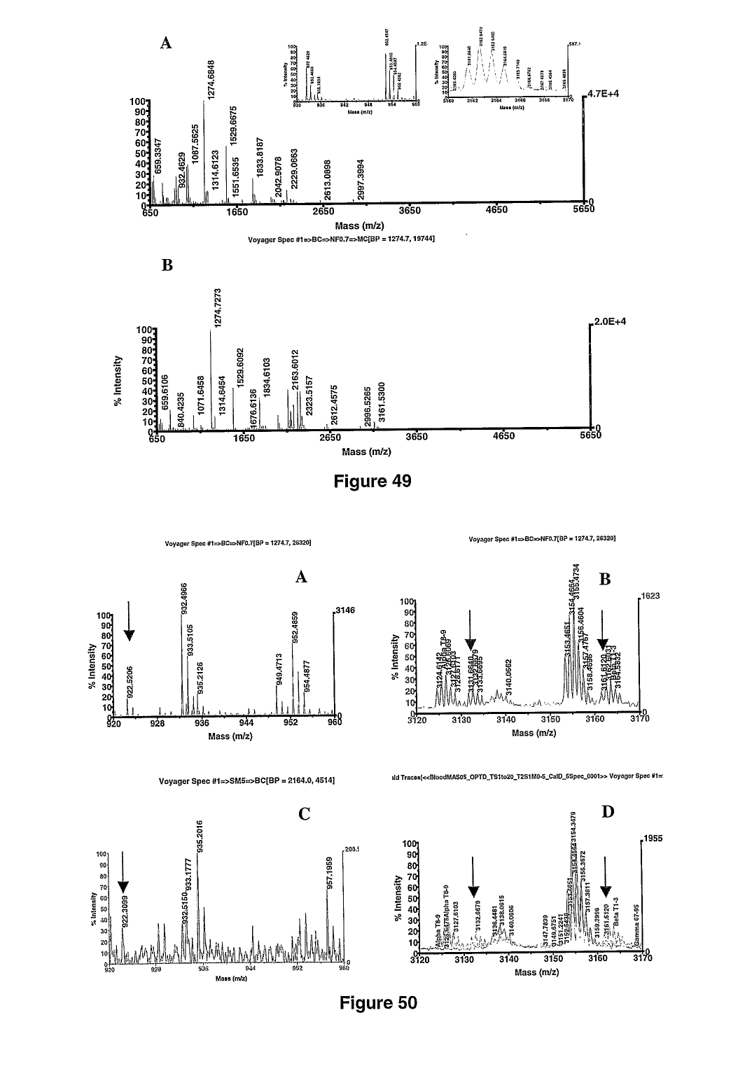

It was observed that, the m/z values of βT1, βT2-3 and βT1-3 are well resolved with tryptic dilutions from 1:20 to 1:100. Spectra obtained using 1:20 dilution of trypsin is shown in FIG. 49A and 1:100 dilution in FIG. 49B. The partially digested fragment βT1-3, with two missed cleavages, was not detected using a 1:10 dilution of trypsin stock solution. The βT1 fragment and βₛT1, m/z value 922.5356 (with a −29.98 mass shift) are the signature peptides for detection of Hb S, and for confirming heterozygous or homozygous state of Hb S, whereby, the detection of the βT1-3 (m/z value 3161.6589) fragment and the βₛT1-3 (m/z value 3131.6847) fragment adds more confidence to the diagnosis. The detection of βT1-2 (m/z value 2228.1669) confirms that the substitution is in βT1 and not in βT1-2. But in incomplete digests, formation of βT1-3 is favoured and T1-2 is favoured, as such the signal for βT1 is weak. In this study, analysis of spectra obtained from the on carrier digests of various dilution of blood sample containing Hb S suggest that detection of βT1, βₛST, βT1-3 and β_{S}T1-3 (m/z 3131.6847) was controlled by the concentration of trypsin when digest time is within 3 minutes, as shown in FIG. 50, all these fragments were detected, with variable intensity, with a trypsin concentration below 5.45 pM/μl. The βT1-3 was detected with same intensity in all MALDI-ToF mass spectra obtained for normal blood sample and sample with Hb S, in all dilution of trypsin stock solution.

It was observed that the number of autolytic tryptic fragments decreased as the dilution factor for trypsin increased. With a fixed on carrier trypsin concentration the number of autolytic fragments increased as the dilution factor for sample increased.

### Example 7

### Sequence Coverage of Blood Collected in a New Sample Collection Procedure

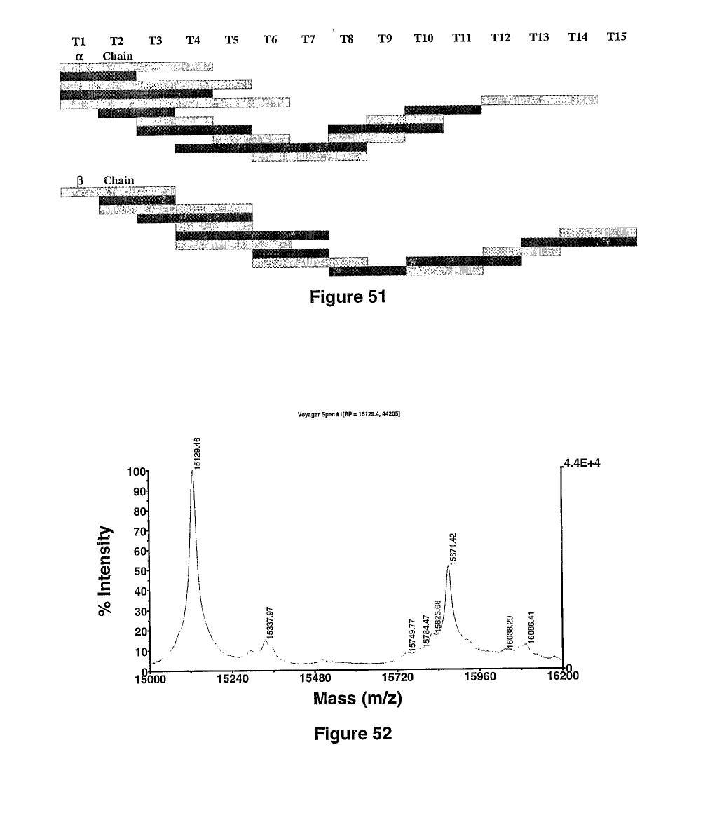

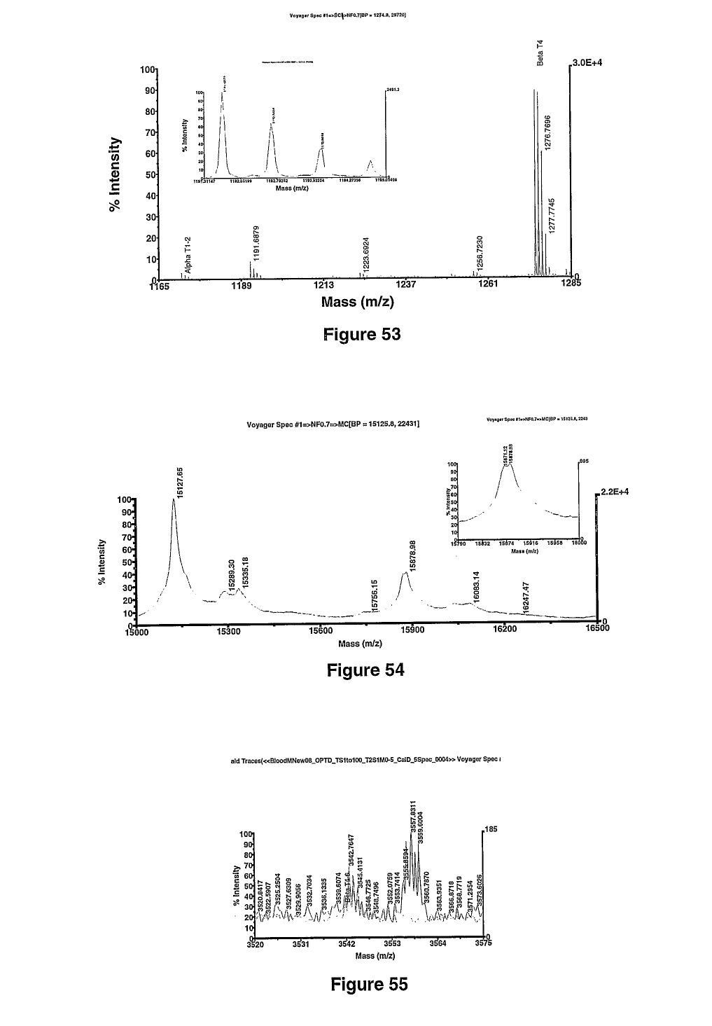

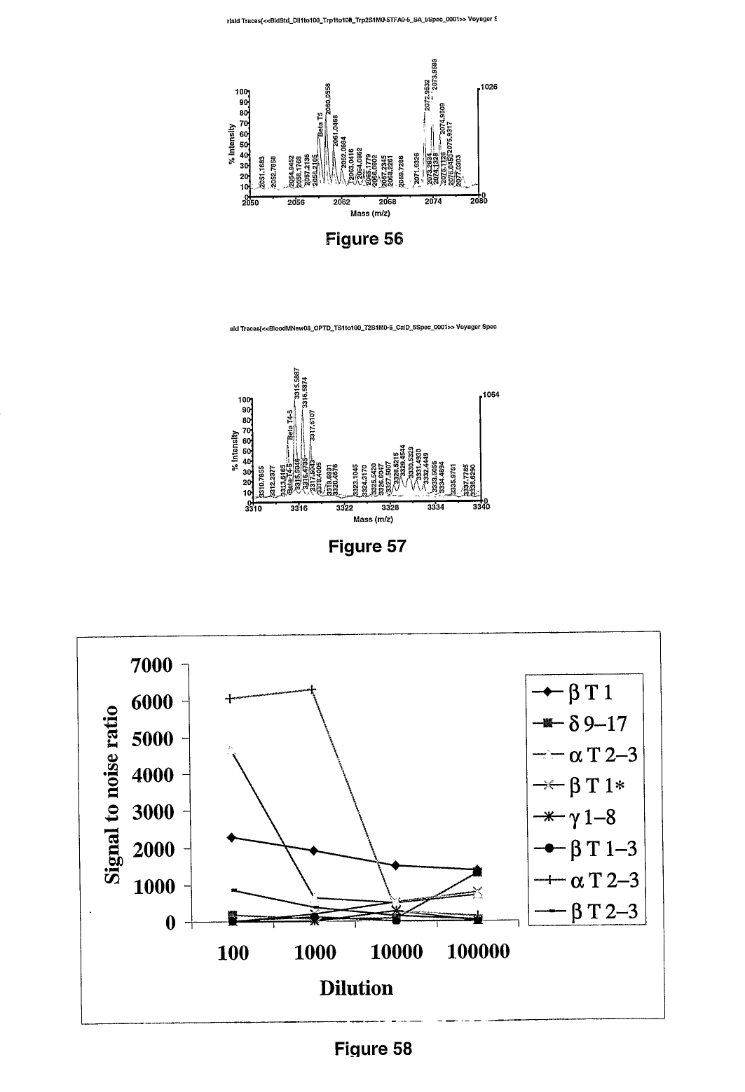

Blood from two individuals having normal Hb directly collected in ammonium bicarbonate buffer was subjected to a 3 min on carrier tryptic digests in the presence of the novel surfactant within a few minutes of sample collection and after three weeks. Similar tryptic fragmentation pattern with similar peak intensities, high ion counts, high mass accuracy and excellent mass resolution were obtained from digests performed of these samples at two different time points. Analysis of mass spectra whereby 10 spectra (each an accumulation of 10 individual spectra, each obtained by 100 laser shots) for each digestion were obtained using MALDI-ToF MS reflector mode show a typical fragmentation pattern, as show in FIG. 51. It is noteworthy from the fragmentation pattern observed for the digests of normal blood that for the α chain, all but the fragments αT11-T15 produced overlapping fragments and for the β chain all except the βT9 produced overlapping tryptic fragments.

Automated data analysis of an MALDI-TOF mass spectra obtained from a 3 min on carrier digest in the presence of the novel surfactant using the Protein Prospector MS Fit option and the SwissPort.r36 database identified 10 α chain fragments and 9 β chain tryptic fragments within the 10 ppm mass accuracy window, as listed in Table 32. The sequence coverage for the α chain was 70% and the β chain 49% with 10 ppm mass accuracy window.

TABLE 32
Mass accuracy of the obtained fragments of α and β chains derived from
an on carrier digestion of whole blood directly collected into ammonium
bicarbonate buffer, 1:100 dilution, at the 3 min time point, with trypsin in
presence of the novel surfactant, in the reflector mode, analysed by the
Protein Prospector software.

| m/z | Mass matched | | | |
|---|---|---|---|---|
| submitted | [m + H]⁺ | Δppm | Position | Fragments |
| 1071.5472 | 1071.5549 | −2.17 | 32-40 | αT5 |
| 1087.6281 | 1087.6264 | 1.52 | 91-99 | αT10-11 |
| 1529.7391 | 1529.7348 | −2.76 | 17-31 | αT4 |
| 1684.9435 | 1684.9386 | 2.90 | 1-16 | αT1-3 |
| 1833.8918 | 1833.8924 | −0.33 | 41-56 | αT6 |
| 2042.9959 | 2043.0048 | −4.34 | 12-31 | αT3-4 |
| 2213.0933 | 2213.0892 | 1.83 | 41-60 | αT6-7 |
| 2341.1817 | 2341.1842 | −1.05 | 41-61 | αT6-8 |
| 2996.4930 | 2996.4900 | 1.01 | 62-90 | αT |
| 3124.5856 | 3124.5850 | 0.2 | 61-90 | αT1-4 |
| 932.5203 | 932.5205 | 0.25 | 9-17 | βT2 |
| 952.5105 | 952.5104 | 0.11 | 1-8 | βT1 |
| 1274.7309 | 1274.7261 | 3.76 | 31-40 | βT4 |
| 1314.6778 | 1314.6654 | 6.11 | 18-30 | βT3 |
| 2058.9612 | 2058.9483 | 6.29 | 41-59 | βT5 |
| 2228.1542 | 2228.1675 | −5.96 | 9-30 | βT2-3 |
| 3161.6502 | 3161.6595 | 2.93 | 1-30 | βT1-3 |
| 3314.6349 | 3314.6560 | −6.35 | 31-69 | βT4-5 |

### Example 8

### Identification of previously unreported Hb Variants

### A. Unstable Hb Variant

A blood sample with abnormal peaks identified employing the standard HPLC method was sent for confirmation of diagnosis by DNA analysis to the Clinical Genetic Laboratory at Monash Medical Centre. The sample was obtained from the Monash Medical Centre haematology laboratory for MALDI-ToF MS analysis.

### Linear Mode Screening

The initial investigation was carried out using the MALDI-ToF MS linear mode. The mass spectrum of obtained from the sample shows the single charged [M+H]⁺ average m/z value 15127.60 (expected m/z value 15127.37) representative for the α chain with associated error was −0.77 Da, as shown in FIG. 52. Whilst investigating the β chain it was observed that an [M+H]⁺ average m/z value of 15869.30 (expected m/z value 15868.23) was observed corresponding the β chain with an associated error of 1.07 Da with three additional [M+H]⁺ average m/z values were observed at 15822.28, 15784.47 and 15746.31 having a mass difference from the β chain (expected m/z value 15868.23) of −45.95 Da, −83.75 Da and −121.92 Da. The appearance of multiple peaks indicated either the presence of multiple amino acid substitutions or presence of an unstable Hb variant.

### On Carrier Trypsin Digestion to Identify the Possible Signature Peptide/s

An on carrier tryptic digest of the blood sample containing the unidentified β chain variant was obtained with the novel ionic surfactant at 37° C. and a 3 min digest time. 10 MALDI-TOF mass spectra, each an accumulation of 5 spectra whereby each spectrum was obtained by 100 laser shots, were obtained from the digests. Automated data analysis of all the spectra using the Protein Prospector MS Fit programme and the SwissPort.r36 database identified 6-9 α chain tryptic fragments and 5-7 β chain tryptic fragments within the 10 ppm mass accuracy window. The best spectrum with the highest number of identified α and β chain tryptic fragment was identified, baseline corrected, noise filter smoothed and peak deisotoped using the Data Explorer ver 4.0.0.0 software. The deisotoped m/z values were then analysed with two automated data analysis procedures, the FindMod option and the Homology option, the latter using the Protein Prospector programme with molecular mass range set to 15500 to 16000 (β chain mass range), pl 6-7, enzyme to trypsin with maximum missed cleavages to 5, number of amino acid substitution to 1, mass accuracy window to 50 ppm and for the homology mode mass shift to −45.95 Da, −83.75 Da and −121.92 Da respectively. The reproducible occurring unassigned m/z values that were observed for all samples investigated in this study were excluded. The filters were set to exclude to tryptic autolytic fragments and keratin artefact peaks. Only one potential signature peptide was identified with a monoisotopic [M+H]⁺ m/z value of 1191.6879, as shown in Table 33.

TABLE 33
Automated report generated by the Protein Prospector software
using the monoisotopic mass list obtained from the 3 min on
carrier digest of whole unpurified blood containing the new variant
in the presence of the novel detergent.

| m/z | MH⁺ | | | | | |
|---|---|---|---|---|---|---|
| submitted | matched | Δppm | start | end | Peptide Sequence | Modifications |
| 932.5265 | 932.5205 | 6.3508 | 9 | 17 | SAVTALWGK | |
| 952.5169 | 952.5104 | 6.8542 | 1 | 8 | VHLTPEEK | |
| 1191.6879 | 1191.6560 | 26.7518 | 31 | 40 | LLVVYPCTQR | W7->C(−83.0701) |
| 1274.7755 | 1274.7261 | 38.7003 | 31 | 40 | LLVVYPWTQR | |
| 1314.7142 | 1314.6654 | 37.1269 | 18 | 30 | VNVDEVGGEALGR | |

As such, an amino acid substitution that causes a mass shift of −83.0643 Da in the βT4 fragment with a resulting m/z value of 1191.6879 was identified solely by automated data analysis. The substitution identified was Trp (W) to Cyc (C) at position 37 of the β chain as shown in Table 34. No other substitutions were identified at this time point. Simultaneous results reported by DNA analysis using a standard method of the sample aided and confirmed the MALDI-TOF MS identification of the new Hb variant. The reported DNA analysis result was that a mutation in codon 37, G→C (TGG→TGC) had occurred. The presence of normal β chain and normal βT4 tryptic fragment confirms the heterozygous state for the variant.

TABLE 34
Identified signature peptide for the previously unreported variant
using the newly devised 3 min on carrier proteolytic digest (trypsin)
in the presence of the novel surfactant, with mass accuracy.

| | | | Missed | Theoretical | Received | |
|---|---|---|---|---|---|---|
| Fragment | Position | Sequence | Cleavage | Mass | Mass | ppm |
| β_{NewM1}T4 | 31-40 | LLVVYPCTQR | 0 | 1274.7261 | 1274.7755 | 38.70 |
| βT4 | 31-40 | LLVVYPWTQR | 0 | 1191.6560 | 1195.6879 | 26.75 |

### B. Unreported New Hb Cariant

A blood sample with a HPLC report showing unusual peaks was obtained from the Monash Medical Centre haematology laboratory for MALDI-ToF MS analysis.

### Linear Mode Screening

Initial investigation carried out using the MALDI-ToF MS linear mode of the sample shows the single charged [M+H]⁺ average m/z value 15127.65 (expected m/z value 15127.37) representative for the α chain with an associated error of −0.28 Da, a [M+H]⁺ average m/z value of 15871.12 (expected m/z value 15868.23) representative for the β chain with an associated error of 2.89 Da and an additional poorly separated peak with a m/z value of 15878.98 Da resulting in a mass shift of 10.75 Da.

### On Carrier Trypsin Digestion to Identify the Possible Signature Peptide/s

An on carrier tryptic digest of the blood sample was performed with the novel ionic surfactant at 37° C. and a 3 min digest time. MALDI-TOF mass spectra were obtained using automated data acquisition and 10 collected spectra were analysed using the Protein Prospector MS Fit programme and the SwissPort.r36 database. The best spectrum with the highest number of identified α and β chain tryptic fragments within a 10 ppm mass accuracy window was identified, baseline corrected, noise filter smoothed and peak deisotoped using the Data Explorer software. The deisotoped m/z values were then analysed with two automated data analysis procedures, the FindMod programme and the homology option, the latter using the Protein Prospector software. The criteria were set to a molecular mass range of 15500 to 16000 (β chain mass range), pl 6-7, enzyme to trypsin with maximum missed cleavages to 5, number of amino acid substitution to 1, a mass accuracy window of 50 ppm and for the homology mode mass shift of 5 to 15 Da. Although the obtained mass difference was 10.75 in the linear mode MALDI mass spectrum, mass shifts within a 5 to 15 Da window were explored assuming a poor separation of the β chain peaks resulted in an error in the mass difference between the normal and variant p globin chains. The reproducible, in all spectra of 1:100 dilution of blood occurring unassigned m/z values, possible tryptic autolytic fragments and keratin artefact peaks were not considered using a filter. The automated data analysis identified a signature peptide with a monoisotopic [M+H]⁺ m/z value of 2072.9705, with 11 possible amino acid substitution for the βT5 tryptic fragment (expected m/z value 2058.9483, received m/z value 2058.9483), as shown in Table 35 corresponding to a 14 Da mass difference.

TABLE 35
Automated report generated by the Protein Prospector software using the
monoisotopic m/z values obtained from a 3 min on carrier digest in the presence of the
novel detergent of whole unpurified blood containing an unreported variant.

| m/z | MH⁺ | | | | | |
|---|---|---|---|---|---|---|
| submitted | matched | Δ ppm | start | end | Peptide Sequence | Modifications |
| 932.5150 | 932.5205 | −5.9516 | 9 | 17 | SAVTALWGK | |
| 952.4988 | 952.5104 | −12.1610 | 1 | 8 | VHLTPEEK | |
| 1274.7190 | 1274.7261 | −5.5856 | 31 | 40 | LLVVYPWTQR | |
| 1314.6616 | 1314.6654 | −2.8458 | 18 | 30 | VNVDEVGGEALGR | |
| 1669.9064 | 1669.8913 | 8.9997 | 67 | 82 | VLGAFSDGLAHLDNLK | |
| 2058.9479 | 2058.9483 | −0.1819 | 41 | 59 | FFESFGDLSTPDAVMGNPK | βT5 |
| 2072.9705 | 2072.9275 | 20.7421 | 41 | 59 | FFESFGDLSDPDAVMGNPK | T10->D |
| | | | | | | (+13.9793) |
| 2072.9705 | 2072.9639 | 3.1894 | 41 | 59 | FFETFGDLSTPDAVMGNPK | S4->T |
| | | | | | | (+14.0157) |
| 2072.9705 | 2072.9639 | 3.1894 | 41 | 59 | FFESFGDLTTPDAVMGNPK | S9->T |
| | | | | | | (+14.0157) |
| 2072.9705 | 2072.9639 | 3.1894 | 41 | 59 | FFESFADLSTPDAVMGNPK | G6->A |
| | | | | | | (+14.0157) |
| 2072.9705 | 2072.9639 | 3.1894 | 41 | 59 | FFESFGDLSTPDAVMANPK | G16->A |
| | | | | | | (+14.0157) |
| 2072.9705 | 2072.9639 | 3.1894 | 41 | 59 | FFESFGDLSTPDAVMGQPK | N17->Q |
| | | | | | | (+14.0157) |
| 2072.9705 | 2072.9639 | 3.1894 | 41 | 59 | FFESFGDLSTPDAIMGNPK | V14->I |
| | | | | | | (+14.0157) |
| 2072.9705 | 2072.9639 | 3.1894 | 41 | 59 | FFESFGELSTPDAVMGNPK | D7->E |
| | | | | | | (+14.0157) |
| 2072.9705 | 2072.9639 | 3.1894 | 41 | 59 | FFESFGDLSTPEAVMGNPK | D12->E |
| | | | | | | (+14.0157) |
| 2072.9705 | 2072.9639 | 3.1894 | 41 | 59 | FFESFGDLSTPDALMGNPK | V14->L |
| | | | | | | (+14.0157) |
| 2072.9705 | 2073.0003 | −14.3628 | 41 | 59 | FFESFGDLSTPDAVMGKPK | N17->K |
| | | | | | | (+14.0520) |
| 2228.1342 | 2228.1675 | −14.9742 | 9 | 30 | SAVTALWGKVNVDEVGGE | |
| | | | | | ALGR | |
| 3161.5897 | 3161.6595 | −22.0835 | 1 | 30 | VHLTPEEKSAVTALWGKV | |
| | | | | | NVDEVGGEALGR | |

In the tryptic fragmentation pattern observed for normal blood in this invention the βT5 tryptic region produced a few overlapping fragments. If a mutation occurred, as it is the case with this mutant, resulting in an amino acid substitution causing a 14 Da mass shift in the βT5 fragment, it is expected that this mass shift is also observed in the fragments with missed cleavages. Manual inspection of the spectra confirmed the presence of βT4-5, the βT4-6 (expected m/z values of 3314.6554 and 3541.8187 respectively) and the additional tryptic fragments resulting from the presence of the mutation namely the βT_{MNO2}4-5 and the βT_{MNO2}4-6 (expected m/z values of 3328.6170 and 3555.8344 respectively), as shown in FIGS. 55, 57 and 58.

The appearance of three signature peptides, as listed in Table 35 confirms the location of the substitution to be in the βT5 tryptic fragment with a mass shift of +14 Da. As such, an amino acid substitution with a list of possible substitution and the location of substitution was identified solely by automated data analysis. From this several possibilities can be excluded. The N→K mutation in not likely, because this would introduce an additional cleavage site and the resulting fragments could not be detected. The D→E mutation can be excluded since the respective fragments could not be detected in the endoproteinase Glu C digests (data not shown). The next step to identify the substitution would have been to perform de novo MS sequencing using CID and PSD analysis. Simultaneous DNA analysis of the sample using standard methods revealed that a mutation at the codon 54, G→C (GTT→CTT) had occurred. The resulting amino acid substitution is Val⁵⁴ (V)→Leu⁵⁴ (L) with a mass shift of +14.0157.

The presence of the normal β chain and the normal counterparts of the identified signature peptides βT5_{NM2}, βT4-5^{NM2} and βT4-6_{NM2} tryptic fragments, as shown in FIG. 55, 56, 57, and Table 36 confirms the heterozygous state for the variant.

TABLE 36
Identified signature peptides for the previously unreported Hb variant using
the newly devised 3 min on carrier proteolytic digest (trypsin) in the presence
of the novel surfactant, with mass accuracy.

| | | | Missed | Theoretical | Received | |
|---|---|---|---|---|---|---|
| Fragment | Position | Sequence | Cleavage | Mass | Mass | ppm |
| β_{NM2}T5 | 41-49 | FFESFGDLSTPD | 0 | 2072.9705 | 2072.9639 | 3.9 |
| | | ALMGNPK | | | | |
| β_{NM2}T4-5 | 31-49 | LLVVYPWTQRFF | 1 | 3328.671 | 3328.5215 | 44.9 |
| | | ESFGDLSTPDAL | | | | |
| | | MGNPK | | | | |
| β_{NM2}T4-6 | 31-61 | LLVVYPWTQRFF | 2 | 3555.8344 | 3555.0594 | 217.5 |
| | | ESFGDLSTPDAL | | | | |
| | | MGNPKVK | | | | |

### Example 9

### Detection of Low Abundance Peptide Fragments

Investigations were carried out to optimise conditions suitable for the detection of very low abundance peptides in a complex mixture of high and low abundance peptides derived from on carrier digests of protein mixtures. Blood contains a complex mixture of Hbs with a high abundance of Hb A (α₂β₂). The Hb A₂, a minor component of adult blood has two δ chains with two α chains (α₂δ₂), and consists of only 2-3% of the total Hb content, where as the Hb F (α₂γ₂), another minor component is present in adults only in trace amounts (less than 1%). The δ chain percentage equals the Hb A₂ percentage. The level of ζ chain in normal newborns averages 0.19% although it varies considerably with ethnicity. Thus, a proteolytic digest of whole blood would yield a very complex mixture of their peptides derived from all the Hb chains with various abundances making the identification of proteolytic peptide fragments very difficult and challenging.

### Investigation into the Detectability of Peptides with Variable Abundance

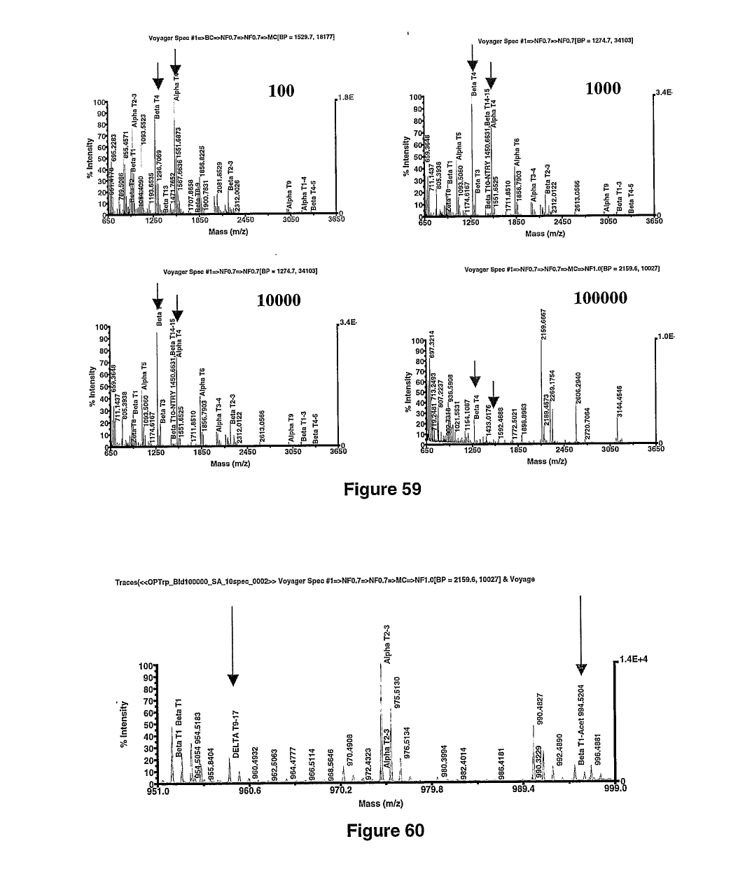

Normal blood with adult Hb diluted 1:100 was incubated with the novel detergent RapiGest™ for 5 minutes and diluted 1:500, 1:1000, 1:5000, 1:10000, 1:50000 and 1:100000 with ammonium bicarbonate buffer followed by the newly developed method for on carrier 3 min proteolytic digestion at 37° C. for each dilution. For each dilution 5 different spectra were accumulated, each with an accumulation of 10 spectra whereby each spectrum was an accumulation of 100 laser shots. All the spectra were thoroughly analysed by visual inspection and automated protein identification using the Protein Prospector software. The appearance and disappearance of certain peaks were monitored for all the dilutions. The signal strength was determined by calculating the signal to noise ratio using the Data Explorer software. The on carrier 3 min tryptic digest at 37° C. in the presence of the novel surfactant RapiGest™ produced strong signals for the αT4, αT2-3 and the βT4 proteolytic fragments (m/z values 1529.7342, 974.5418 and 1274.7255). Initially, these three peaks were monitored for their appearances for all the dilutions. All three the peaks were detectable with confidence for dilutions as high as 100000, although the signal strength gradually decreased, as shown in Table 37, FIGS. 58 and 59. The signal to noise ratio of the peaks decreased from high (6000) to low (100) for dilutions 1:100 to 1:100000, as depicted in Table 37 and FIG. 58. Three comparatively low abundance peaks, βT1, βT2-3 and βT1-3, were targeted in the second phase of the analysis whereby it was observed that the m/z values of βT1 and βT2-3 were resolved for all dilutions with the signal to noise ratio decreasing drastically with dilutions higher than 10000, as shown in Table 37 and FIG. 58. The βT1-3 could not be detected in dilutions above 1: 5000 (data not shown). Surprisingly the acetylated βT1 fragment was observed in all dilutions above 1:100, as shown in FIG. 60.

TABLE 37
Obtained signal to noise ratios of peaks at different dilutions of
the blood sample using the MALDI-ToF MS reflector mode.

Signal to noise ratio (Dilutions)

| Chain | 100 | 1000 | 10000 | 100000 |
|---|---|---|---|---|
| βT1 | 2287.3 | 1920.6 | 1508.7 | 1375.8 |
| βT4 | 4335.80 | 7208.00 | 2347.70 | 527.20 |
| βT2-3 | 866.60 | 384.60 | 146.90 | 47.10 |
| βT1-3 | 13.40 | 123.00 | 0.00 | 0.00 |
| αT2-3 | 4675.1 | 643.8 | 500 | 716.3 |
| αT4 | 6064.50 | 6295.00 | 265.60 | 139.70 |
| βT1* | 0 | 203.9 | 521.3 | 793.3 |
| δ9-17 | 181.2 | 68.9 | 78.1 | 1307.2 |
| γ1-8 | 0 | 0 | 282.7 | 0 |

*Acetylated

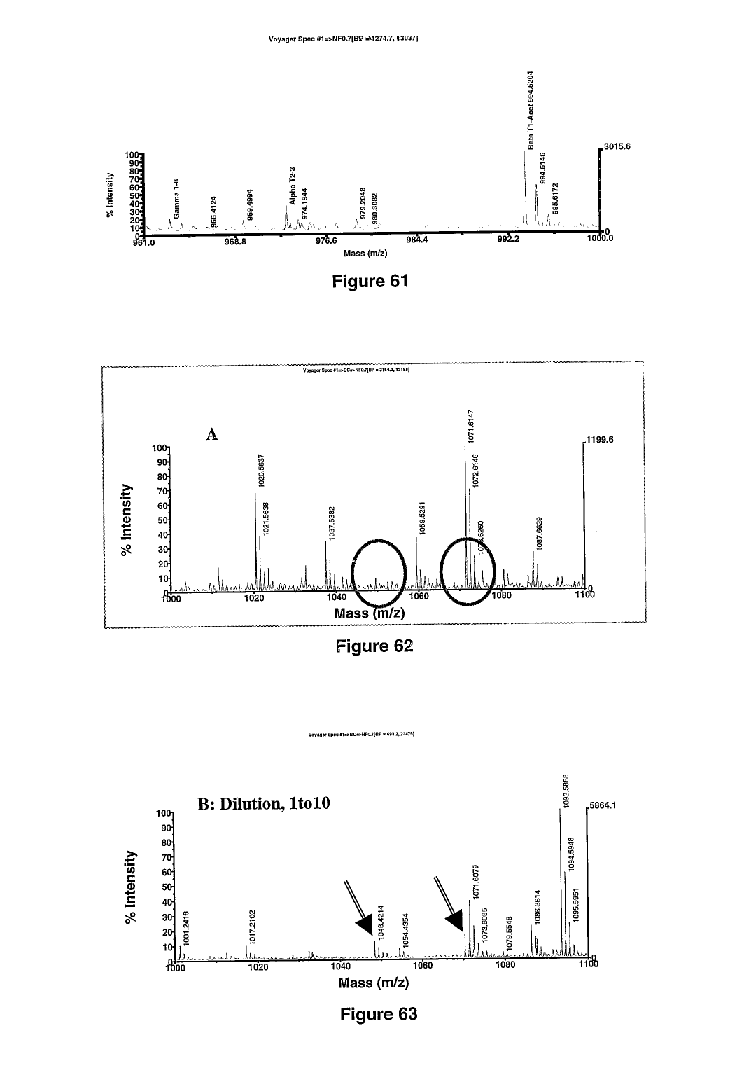

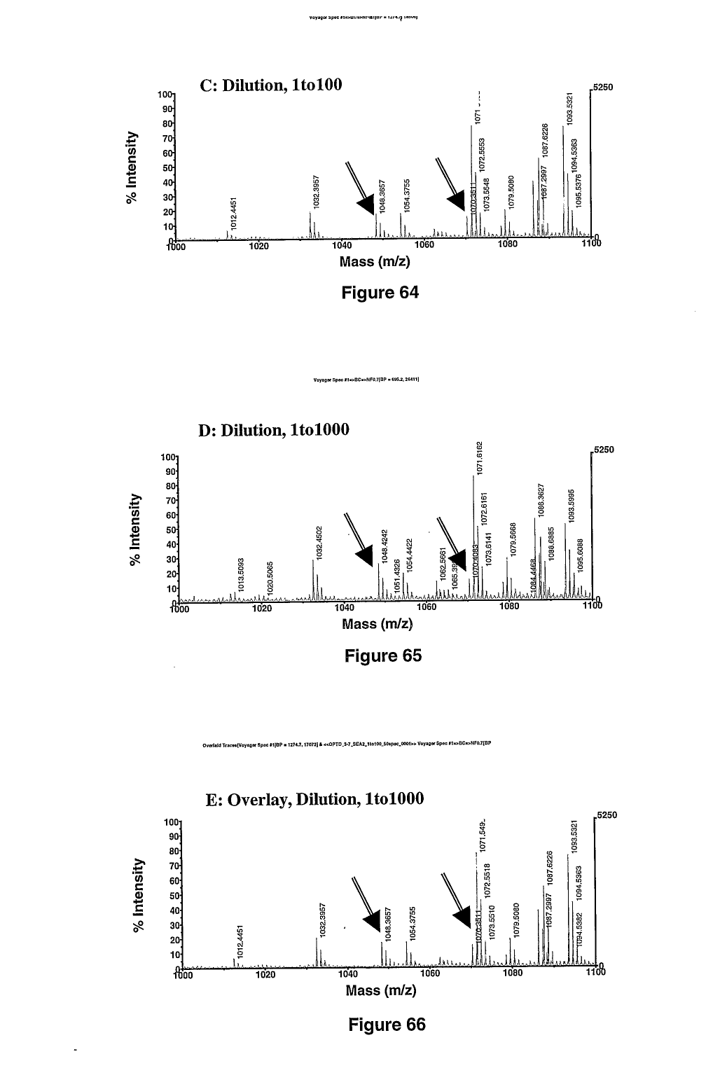

In this invention the 69-17 fragment was monitored to monitor the effect of the dilution factor on a low abundance Hb A₂ fragment. It was interesting to observe, that the signal strength for the peak gradually increased as the dilution factor was increased reaching its highest strength in the 1:100000 dilution, as shown in Table 37 and FIG. 60. The most interesting finding was the appearance of a γ globin chain fragment in the 1:10000 dilution whereby the appearance of the peak was reproducible for this dilution factor as shown in Table 37 and FIG. 61.

### Example 10

### Detection of Haemoglobin ζ Chain in Patients with α Thalassaemia

Three different dilutions of blood samples obtained from three patients having α gene deletions --/αα(-α^{3.7}/-α^{3.7}, -α^{3.7}/--^{SEA}) and one normal Hb from blood of a healthy individual, 1:10, 1:100 and 1:1000 with ammonium bicarbonate buffer, were investigated. The on carrier trypsin digestion of these samples was performed with the presence of the ionic surfactant RapiGest™ SF at 37° C. and a 3 min digest time. For each sample, 10 accumulations, each for 5 and 50 spectra, were obtained. Each spectrum was obtained by 100 laser shots (laser intensity set to 2400), and accumulated using selection criteria of a minimum resolution of 10000, a minimum signal intensity of 1000 and a maximum signal intensity of 64000 for the base peak, βT4 (1274-1275). All spectra were analysed using the ProteinProspector software, and for the automated detection of Hb ζ chain, the pre-processing filter was set to a mass accuracy of 400 ppm and the post-processing filter was set to a final mass accuracy of 250 ppm, the mass range to 5000-16500 Da, and the pl to 6.5-9. The results obtained for the two α gene deletion samples of three different dilutions were compared against the normal.

Analysis of the obtained spectra of the samples, as shown in Table 38, demonstrate that with the condition applied in this invention, the detection of the following ζ tryptic fragments were possible, with increasing mass, ζT8 (m/z 928.5642), ζT3 (m/z 1048.5859), ζT5 (m/z 1070.5993), ζT9 (m/z 1075.5629), ζT6 (m/z 1885.9343) and ζT14 (m/z 1308.7409). Since the αT11 and the ζT11 both have the same amino acid composition, and as such posses the same m/z value, 818.4406, it was not considered as a diagnostic fragment, although it was detected.

TABLE 38
The detection of Hb ζ chain fragments with
MALDI-ToF mass spectrometry.

| Identified ζ | | Possible conflicts: |
|---|---|---|
| chain fragments | m/z | Identical m/z |
| ζT11 | 818.4406 | αT11 (identity) |
| ζT8 | 928.5642 | |
| ζT3 | 1048.5859 | |
| ζT5 | 1070.5993 | |
| ζT9 | 1075.5629 | |
| ζT6 | 1885.9343 | |
| ζT14 | 1308.7409 | |

Homology between [symbol] and [symbol]
Identical fragments: αT11 and ζT11 (818.4406), αT14 and ζT15 (338.1823)

TABLE 39
The detection of haemoglobin δ chain fragments
with MALDI-ToF mass spectrometry.

| | | Possible | | |
|---|---|---|---|---|
| Identified δ | | conflicts: | Possible conflicts: | Missed |
| chains | m/z | Identical m/z | Similar m/z | Cleavage/s |
| δT15 | 1149.7961 | βT14 | | |
| δT3 | 1256.6593 | | | |
| δT4 | 1274.7255 | βT4, εT4, γT4 | | |
| δT14 | 1441.6780 | | | |
| δT15-16 | 1449.7961 | βT14-15, | γT13 (1449.7008) | 1 |
| δT9 | 1669.8907 | βT9 | | |
| δT8-9 | 1797.9857 | βT8-9 | | 1 |
| δT13-14 | 1887.9058 | | | 1 |
| δT2-3 | 2197.1723 | | | 1 |
| δT14-15 | 3018.5618 | | | 1 |

Some peptide fragments derived from the minor Hb fractions, the γ and [symbol] chains, were also detected. The detected δ chain fragments, derived from minor Hb component A₂, with increasing mass, were δT3 (m/z 1256.6593), δT14 (m/z 1441.6780), δT13-14 (m/z 1887.9058), δT2-3 (m/z 2197.1723) and δT114-15 (m/z 3018.5618). The δT15 (m/z 1149.7961.) which has an identical m/z value as βT14, the δT4 having a identical m/z value with βT4/εT4/γT4 (m/z 1274.7255), δT9 (m/z 1669.891) with βT9, δT14-15 with a m/z value similar to βT14-15 (1449.7961 and 1449.008 respectively), δT9 identical with βT9 (m/z 1669.8907) and δT8-9 identical with βT8-9 (m/z 1797.9857) were also detected, as shown in Table 39.

The detected y chain fragments identified unambiguously, derived from Hb component F, present in trace amount in adults, with increasing mass, were the γT1 (m/z 1093.4624 with Met^{INI}) and the γT12 (m/z 3124.7193). The γT111 fragment (m/z 1098.5578) is identical to the εT11, the γT4 having an identical m/z value with βT4/εT4 (m/z 1274.7255), the εT2-3 with a m/z value similar to the δT5-6 (2274.1724 and 2272.0954 respectively) were also detected, as shown in Table 40.

TABLE 40
The detection of Hb γ chain fragments with MALDI-ToF mass spectrometry.

| Identified | m/z | Possible conflicts: | Possible conflicts: | Missed | Additional |
|---|---|---|---|---|---|
| γ chains | values | Identical m/z | Similar m/z | Cleavage/s | Information |
| γT1 | 1093.4624 | | | 1 | with Met^{INI} |
| γT11 | 1098.5578 | εT11 | | 0 | |
| γT4 | 1274.7255 | βT4, εT4, γT4 | | 0 | |
| γT13 | 1449.7008 | | δT15-16 | 0 | |
| | | | (1449.7961) | | |
| | | | βT14-15 | | |
| | | | (1449.7961) | | |
| γT2-3 | 2274.1724 | | δT5-6 | 1 | |
| | | | (2272.0954) | | |
| γT12 | 3124.7193 | | | 0 | |

After automated analysis and detection of peaks, all the spectra were manually inspected to confirm the presence of the respective peak. The comparison of the 50 accumulated spectra with 5 accumulated spectra show that an increased number of ζ chain fragments were identified with greater dilution of the sample, in particular the 1:1000 dilution, and that the ζT3 and the ζT5 were identified in all three samples with α thalassaemia in all dilutions when 50 spectra were accumulated, as shown in Tables 41 and 42. The mass accuracy of the identified ζ chain fragments was low, which is expected because of the extremely low abundance of the ζ chain fragment ions. The presence of the ζT3 and the ζT5 in all three dilutions when 50 spectra were accumulated are shown in FIG. 63, 64, 65. The accumulation of 5 spectra failed to resolve these fragments at times signifying the spot to spot variance of the presence of the same fragment. Most importantly, however was the absence of any ζ fragments in the normal blood sample spectra, as shown in FIGS. 62 and 66. The detection of ζ fragments in all three samples with two ζ gene deletion samples is in agreement with the reported elevation of embryonic ζ chain level in adult carriers of two α gene deletion.

TABLE 41
Automated detection of Hb tryptic fragments with 5 accumulated spectra.

| | | | |
|---|---|---|---|
| | −α^{3.7}/—^{SEA} | −α^{3.7}/—^{SEA} | −α^{3.7}/−α^{3.7} |
| | Identified | Identified | Identified |
| Normal Blood | Fragment (Δppm) | Fragment (Δppm) | Fragment (Δppm) |

1:10 Dilution

| | | |
|---|---|---|
| [symbol] | 8 frag. (0.7-31.2) | 7 frag. (0-208.9) |
| [symbol] | 6 frag. (14.2-200.7) | 7 frag. (0-55.7) |
| [symbol] | δT15 (200.7) | δT4 (4.4) |
| | δT4 (17.7) | δT15-16 (0.7) |
| | δT15-16 (26.3) | |
| [symbol] | γT1 Met^{INI} (78.4) | γT1 Met^{INI} (64.8) |
| | γT4 (17.6) | γT4 (4.4) |
| | γT13 (92.0) | γT13 (65.0) |
| [symbol] | ζT3 (202.9) | ζT3 (206.9) |
| | ζT5 (−200.0) | ζT5 (229.7) |
| | ζT14 (120.3) | ζT14 (104.5) |

1:100

| | | | | |
|---|---|---|---|---|
| [symbol] | 13 frag. (0.9-55.6) | 9 frag. (0.1-58) | 8 frag. (0.8-49.5) | 13 frag. (1.1-199.2) |
| [symbol] | 6 frag. (2.3-9.7) | 7 frag. (2.0-15.8) | 7 frag. (0.1-8.3) | 7 frag. (1.7-8.1) |
| [symbol] | δT3 (34.5) | δT3 (9.9) | δT3 (52.3) | δT3 (37.5) |
| | δT4 (4.8) | δT4 (2.2) | δT4 (2.3) | δT4 (2.6) |
| | δT2-3 (2.7) | δT15-16 (8.4) | δT15-16 (2.9) | δT15-16 (3.6) |
| [symbol] | γT1 Met^{INI} (68.2) | γT1 Met^{INI} (65.6) | γT1 Met^{INI} (68.5) | γT1 Met^{INI} (59.2) |
| | γT4 (4.8) ( | γT4 (2.2) | γT4 (2.3) | γT4 (2.6) |
| | γT12 (42.3) | γT13 (74.1) | γT13 (68.7) | γT13 (69.3) |
| [symbol] | | | ζT11 (m242) | |
| | | | ζT3 (m194) | |
| | | | ζT5 (m 26.8) | |

1:1000

| | | | |
|---|---|---|---|
| αchain | | 7 frag. (2.6-52.5) | 7 frag. (0-48.0) |
| [symbol] | 8 frag. (0.5-14.3) | 6 frag. (1.8-27.2) | 7 frag. (0.5-81.8) |
| [symbol] | δT4 (2.5) | δT15 (210.9) | δT15 (81.8) |
| | δT14 (73.7) | δT4 (2.4) | δT4 (0.5) |
| | δT15-16 (0.5) | δT15-16 (3.7) | δT15-16 (1.8) |
| | δT9 (1.7) | | |
| [symbol] | γT1 Met^{INI} (70.1) | γT1 Met^{INI} (61.3) | γT1 Met^{INI} (69.7) |
| | γT4 (2.5) | γT4 (2.4) | γT11 (185.4) |
| | γT13 (65.2) | γT13 (69.4) | γT4 (0.5) |
| | | | γT13 (67.5) |
| [symbol] | ζT11 (15.5615) | ζT11 (3.6487) | ζT11 (27.4649) |
| | ζT8 (57.8242) | ζT8 (−84.1381) | ζT3 (196.9433) |
| | ζT3 (210.0191) | ζT3 (219.6242) | ζT5 (229.9273) |
| | ζT5 (234.1768) | ζT5 (236.5258) | |
| | ζT14 (50.1750) | | |

TABLE 42
Automated detection of Hb tryptic fragments with 50 accumulated spectra.

| | | | |
|---|---|---|---|
| | −α^{3.7}/—^{SEA} | −α^{3.7}/—^{SEA} | −α^{3.7}/−α^{3.7} |
| | Identified | Identified | Identified |
| | Fragment | Fragment | Fragment |
| Normal Blood | (Δppm) | (Δppm) | (Δppm) |

1:10 Dilution

| | | | | |
|---|---|---|---|---|
| [symbol] | 8 frag. (2.9-46.5) | No protein found | 8 frag. (1.1-50.9) | 8 frag. (0.1-51.7) |
| [symbol] | 3 frag (40.2-47.4) | | 6 frag. (0.1-202.2) | 7 frag. (2.3-183.9) |
| [symbol] | | δT15 (202.2) | δT15 (184.0) | |
| | | δT4 (0.2) | δT4 (6.1) | |
| | | δT15-16 (0.3) | δT15-16 (2.3) | |
| [symbol] | T1 Met^{INI} (10.7) | T1 Met^{INI} (67.0) | T1 Met^{INI} (66.1) | |
| | γT4 (43.2) | γT4 (0.2) | γT4 (6.1) | |
| | γT13 (113.1) | γT13 (65.4) | γT13 (63.4) | |
| [symbol] | Not found! | ζT3 (205.6) | ζT3 (186.3) | ζT3 (m43.9) |
| | | ζT5 (227.9) | ζT5 (213.8) | ζT5 (m79.9) |
| | | ζT14 (124.8) | ζT14 (91.1) | |
| | | | ζT6 (34.5) | |

1:100

| | | | | |
|---|---|---|---|---|
| [symbol] | 12 frag. (0.2-52.4) | 9 frag. (0.1-52.3) | 8 frag. (0.1-51.9) | 11 frag. (0.1-48.1) |
| [symbol] | 6 frag. (4.1-11.4) | 7 frag. (0-10.9) | 7 frag. (0.4-8.0) | 7 frag. (1.1-6.8) |
| [symbol] | | δT3 (22.4) | | |
| | | δT4 (7.7) | | |
| | | δT15-16 (6.6) | | |
| | | δT14-15 (34.6) | | |
| [symbol] | γT1 Met^{INI} (72.2) | T1 Met^{INI} (68.7) | T1 Met^{INI} (69.0) | T1 Met^{INI} (63.8) |
| | γT13 (3.7) | γT4 (7.7) | γT4 (1.5) | γT4 (1.2) |
| | γT12 (42.2) | γT13 (72.3) | γT13 (68.1) | γT13 (67.2) |
| [symbol] | | ζT11 (m103) | ζT11 (m74.2) | ζT11 (m438) |
| | | ζT3 (m 586) | ζT3 (m210) | ζT3 (m140) |
| | | | ζT5 (m232) | ζT5 (m136) |

1:1000

| | | | |
|---|---|---|---|
| [symbol] | 9 frag. (0.9-43.2) | 7 frag. (0.4-232.4) | 7 frag. (0.1-47.6) |
| [symbol] | 9 frag. (2.0-10.6) | 7frag. (0.4-78.1) | 5 frag. (0.9-79.1) |
| [symbol] | δT4 (1.2) | δT15 (78.1) | |
| | δT14 (80.3) | δT4 (0.4) | |
| | δT15-16 (6.5) | δT15-16 (7.2) | |
| | δT9 (3.6) | | |
| | δT8-9 (10.4) | | |
| [symbol] | δT1 Met^{INI} (71.2) | T1 Met^{INI} (70.9) | |
| | δT4 (1.2) | γT4 (0.4) | |
| | δT13 (72.2)) | γT13 (72.9) | |
| [symbol] | ζT11 (6.7) | ζT11 (m10.1) | ζT11 (9.6) |
| | ζT3 (204.2) | ζT3 (m203) | ζT8 (58.8) |
| | ζT5(227.6) | ζT5 (m223) | ζT3 (202.5) |
| | | | ζT5 (227.6) |
| | | | ζT14 (51.3) |

TABLE 43
Cost analysis (AUD) of different diagnostic tools for Hb disorders.

| Test Name | Sample Amount | Cost | Time Taken |
|---|---|---|---|
| HPLC | 1 ml | ~$70 | >4 hours* |
| | | | for 10-20 samples |
| DNA | 2 ml | ~$400 | 5 to >10 days* |
| | | | 8-16 samples/batch |

*Bowden, personal communication, Clinical Genetics Laboratory and Haematology Laboratory, Monash Medical Centre, Clayton, Victoria, Australia.

Finally it is to be understood that various other modifications and/or alterations may be made without departing from the spirit of the present invention as outlined herein.

### BIBLIOGRAPHY

Lapolla, A., Fedele, D., Plebani, M., Aronica, R., Garbeglio, M., Seraglia, R., D'Alpaos, M. and Traldi, P. (1997) A highly specific method for the characterization of glycation and glyco-oxydation products of globins. Rapid Communications in Mass Spectrometry, 11, 613-617.
Lapolla, A., Fedele, D., Plebani, M., Aronica, R., Garbeglio, M., Seraglia, R., D'Alpaos, M. and Traldi, P. (1999) Evaluation of glycated globins by matrix assisted laser desorption/ionisation mass spectrometry. Clin. Chem., 45, 288-290.
Huisman, T. H. J. (1997) Hb E and α-thalassemia; variability in the assembly of β E chain containing tetramers. Hemoglobin, 21, 227-236.
Shapiro, R., McManus, M., Zolut, C. and Bunn, H. (1980) Sites of non-enzymatic glycosylation of human hemoglobin A. J. Biol. Chem., 255, 3120-3127.

1. A method of preparing a sample for MALDI-TOF MS analysis including the steps of:
applying a material to be analysed to a carrier, wherein the material to be analysed includes a liquid component;
removing at least a portion of the liquid component; and
applying a MALDI matrix over the material to be analysed.

2. A method according to claim 1, wherein the step of applying the material is performed by a “spotting” technique.

3. A method according to claim 1 or 2, wherein the material to be analysed includes a biological material or is derived from a biological material.

4. A method according to claim 3, wherein the biological material is selected from the group consisting of: blood, cerebrospinal fluid, urine, saliva, seminal fluid and sweat.

5. A method according to claim 3 or 4, wherein the biological material includes a polypeptide.

6. A method according to claim 5, wherein the polypeptide is a haemoglobin polypeptide or a fragment or variant or a haemoglobin peptide containing a covalently bonded adduct thereof.

7. A method according to claim 6, wherein the haemoglobin polypeptide includes one or more haemoglobins selected from the group consisting of: α, β, γ, δ, ε and ζ haemoglobin.

8. A method according to any one of claims 3 to 7 wherein the material to be analysed is a dilute solution of biological material in water.

9. A method according to claim 8 wherein the biological material has been diluted by a factor of from 1:10 to 1:10000.

10. A method according to claim 8 or 9 wherein the dilute solution contains a buffer.

11. A method according to claim 10 wherein the buffer is ammonium bicarbonate.

12. A method according to any one of claims 1 to 11 wherein the amount of material applied is from 0.1 to 10 μl.

13. A method according to any one of claim 1 to 12 wherein the step of removing a portion of the liquid component is performed in a manner that does not destroy compounds within the material.

14. A method according to claim 13 wherein the step of removing a portion of the liquid component is performed by a method selected from the group consisting of: applying an elevated temperature; reducing air pressure; passing a stream of gas over the surface of the applied material; allowing the applied material to sit at ambient temperature and pressure for a sufficient time for the liquid to be removed by evaporation; or a combination thereof.

15. A method according to claim 13 or 14, wherein at least 50% of the liquid component is removed.

16. A method according to claim 13 or 14, wherein at least 75% of the liquid component is removed.

17. A method according to claim 13 or 14, wherein at least 90% of the liquid component is removed.

18. A method according to claim 13 or 14, wherein removal of the liquid component continues until the material is at least substantially dry.

19. A method according to any one of claims 1 to 18, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid, caffeic acid, 2,4,6-trihydroxyacetophenone (THAP) and 3-hydroxypicolinic acid (HPA), Anthranilic acid, Nicotinic acid, Salicylamide and mixtures thereof.

20. A method according to claim 19 wherein the ratio of MALDI matrix to material to be analysed is from 0.1:1 to 10:1.

21. A method according to any one of claims 1 to 18, further including the step of treating the material to be analysed to partially digest polypeptides within the material.

22. A method according to claim 21, wherein the treatment includes contacting the material to be analysed with a proteolytic agent.

23. A method according to claim 22, wherein the step of contacting the material to be analysed with a proteolytic agent is carried out prior to the step of applying the material to the carrier.

24. A method according to claim 23, wherein the contacting is carried out for a period of from 1 to 24 hours.

25. A method according to claim 21, wherein the step of treating the material to be analysed is carried out on the carrier.

26. A method according to claim 25 wherein the treating involves contacting the material with a proteolytic agent.

27. A method according to claim 26, wherein the step of treating is carried out for from 10 to 3600 seconds.

28. A method according to any one of claims 22 to 27, wherein the proteolytic agent is a protease.

29. A method according to claim 28, wherein the protease is selected from the group consisting of: trypsin and endoprotease Glu C.

30. A method according to any one of claims 21 to 29, wherein the step of treating is carried out in the presence of a surfactant.

31. A method according to claim 30, wherein the surfactant is sodium 3-[(2-methyl-2-undecyl-1,3-dioxolan-4-yl)methoxy]-1-propane-sulfonate.

32. A method according to any one of claims 21 to 31, wherein the step of treating is stopped by the addition of a diluted acid.

33. A method of preparing a sample for MALDI-TOF MS analysis, said sample including a material to be analysed and a carrier, the method including the step of:
conducting an on carrier digestion of a polypeptide within the material.

34. A method according to claim 33, wherein the material to be analysed includes a biological material or is derived from a biological material.

35. A method according to claim 34, wherein the biological material is selected from the group consisting of: blood, cerebrospinal fluid, urine, saliva, seminal fluid and sweat.

36. A method according to claim 34 or 35, wherein the biological material includes a polypeptide.

37. A method according to claim 36, wherein the polypeptide is a haemoglobin polypeptide or a fragment or variant or a haemoglobin peptide containing a covalently bonded adduct thereof.

38. A method according to claim 37, wherein the haemoglobin polypeptide includes one or more haemoglobins selected from the group consisting of: α, β, γ, δ, ε and ζ haemoglobin.

39. A method according to any one of claims 33 to 38, wherein the material may be analysed is applied to the carrier by a spotting technique.

40. A method according to claim 39, wherein the material to be analysed is diluted with a liquid before being applied to the carrier.

41. A method according to claim 40, wherein the liquid includes a buffer.

42. A method according to claim 41 wherein the buffer is ammonium bicarbonate.

43. A method according to any one of claims 33 to 43, wherein the step of conducting an on carrier digest involves contacting the material with a proteolytic agent.

44. A method according to claim 43, wherein the proteolytic agent is applied to the carrier either prior to, simultaneously with, or following the addition of the material to be analysed.

45. A method according to claim 43 or 44, wherein the proteolytic agent is a protease.

46. A method according to claim 45, wherein the protease is selected from the group consisting of: trypsin and endoprotease Glu C.

47. A method according to any one of claims 32 to 46, wherein the step of conducting an on carrier digestion is carried out in the presence of a surfactant.

48. A method according to claim 47, wherein the surfactant is sodium 3-[(2-methyl-2-undecyl-1,3-dioxolan-4-yl)methoxy]-1-propane-sulfonate.

49. A method according to any one of claims 33 to 48, wherein the on carrier digestion results in at least a partial digestion of the polypeptide.

50. A method according to any one of claims 33 to 49, wherein the step of conducting an on carrier digestion is carried out for a period of from 10 to 3600 seconds.

51. A method according to any one of claims 33 to 50, wherein the step of conducting an on carrier digestion is stopped by the addition of a diluted acid.

52. A method according to any one of claims 33 to 50, wherein the step of conducting an on carrier digestion is stopped by the addition of a MALDI matrix over the material.

53. A method according to claim 52, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid; caffeic acid; 2,4,6-trihydroxyacetophenone; 3-hydroxypicolinic acid; Anthranilic acid; Nicotinic acid; Salicylamide and mixtures thereof.

54. A sample for analysis including:
a carrier having a surface;
a layer including a material to be analysed; and
a single MALDI matrix layer;
wherein the layer including the material to be analysed is located between the carrier surface and the MALDI matrix layer.

55. A sample according to claim 54, wherein the sample to be analysed includes a biological material or is derived from a biological material.

56. A sample according to claim 55, wherein the biological material is selected from the group consisting of: blood, cerebrospinal fluid, urine, saliva, seminal fluid and sweat.

57. A sample according to claim 55 or 56, wherein the biological material includes a polypeptide.

58. A sample according to claim 57, wherein the polypeptide is a haemoglobin polypeptide or a fragment or variant or a haemoglobin peptide containing a covalently bonded adduct thereof.

59. A sample according to claim 58, wherein the haemoglobin polypeptide includes one or more haemoglobins selected from the group consisting of: α, β, γ, δ, ε and ζ haemoglobin.

60. A sample according to any one of claims 54 to 59, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid; caffeic acid; 2,4,6-trihydroxyacetophenone; 3-hydroxypicolinic acid; Anthranilic acid; Nicotinic acid; Salicylamide and mixtures thereof.

61. A method of digesting polypeptides within a material including the step of:
conducting the digestion in the presence of sodium 3-[(2-methyl-2-undecyl-1,3-dioxolan-4-yl)methoxy]-1-propane-sulfonate or a derivative thereof.

62. A method according to claim 61, wherein the step of digestion is carried out by a proteolytic enzyme.

63. A method according to claim 62, wherein the proteolytic enzyme is selected from the group consisting of: trypsin and endoprotease Glu C.

64. A method of analysing a polypeptide including the steps of:
partially digesting the polypeptide; and
subjecting the digested polypeptide to MALDI-ToF MS analysis to identify digestion fragments characteristic of the polypeptide.

65. A method according to claim 64, wherein the step of partially digesting the polypeptide is carried out by contacting the polypeptide with a proteolytic agent.

66. A method according to claim 65, wherein the proteolytic agent is selected from the group consisting of: trypsin and endoprotease Glu C.

67. A method according to any one of claims 64 to 66, wherein the step of partially digesting the polypeptide is carried out in solution.

68. A method according to claim 67, wherein the step of partially digesting the polypeptide is carried out for from 1 to 24 hours.

69. A method according to claim 68 wherein following digestion the material is applied to a carrier.

70. A method according to any one of claims 64 to 66, wherein the step of partially digesting the polypeptide is carried out on a carrier.

71. A method according to claim 70, wherein the step of partially digesting the polypeptide is carried out for from 10 to 3600 seconds.

72. A method according to any one of claims 64 to 71, wherein the step of partially digesting the polypeptide is carried out in the presence of sodium 3-[(2-methyl-2-undecyl-1,3-dioxolan-4-yl)methoxy]-1-propane-sulfonate or a derivative thereof.

73. A method according to any one of claims 64 to 72, wherein the digestion is stopped by the addition of a diluted acid.

74. A method according to any one of claims 69 to 73, further including the step of removing a portion of the liquid component of the material, wherein the step of removing the portion of the liquid component is performed in a manner that does not destroy compounds within the material and partially dries the material.

75. A method according to claim 74, wherein the step of removing a portion of the liquid component is performed by a method selected from the group consisting of: applying an elevated temperature; reducing air pressure; passing a stream of gas over the surface of the applied material; allowing the applied material to sit at ambient temperature and pressure for a sufficient time for the liquid to be removed by evaporation; or a combination thereof.

76. A method according to claim 74 or 75, wherein at least 50% of the liquid component is removed.

77. A method according to claim 74 or 75, wherein at least 75% of the liquid component is removed.

78. A method according to claim 74 or 75, wherein at least 90% of the liquid component is removed.

79. A method according to claim 74 or 75, wherein removal of the liquid component continues until the material is at least substantially dry.

80. A method according to any one of claims 69 to 79 further including addition of a MALDI matrix over the material.

81. A method according to claim 80, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid; caffeic acid; 2,4,6-trihydroxyacetophenone; 3-hydroxypicolinic acid; Anthranilic acid; Nicotinic acid; Salicylamide and mixtures thereof.

82. A method of determining the identity of a polypeptide in a material including the steps of:
partially digesting the material;
analysing the digested material by MALDI-ToF MS to determine digestion fragments; and
comparing the digestion fragments with known polypeptide digestion fragments to determine the identity of the polypeptide.

83. A method according to claim 82, wherein the material includes a biological material or is derived from a biological material.

84. A method according to claim 83, wherein the biological material is selected from the group consisting of: blood, cerebrospinal fluid, urine, saliva, seminal fluid and sweat.

85. A method according to any one of claims 82 to 84, wherein the polypeptide is a haemoglobin polypeptide or a fragment or variant thereof.

86. A method according to claim 85, wherein the haemoglobin polypeptide includes one or more haemoglobins selected from the group consisting of: α, β, γ, δ, ε and ζ haemoglobin.

87. A method according to any one of claims 82 to 86, wherein the step of partially digesting the material includes contacting the material with a proteolytic agent.

88. A method according to claim 87, wherein the proteolytic agent is a protease.

89. A method according to claim 88, wherein the protease is selected from the group consisting of: trypsin and endoprotease Glu C.

90. A method according to any one of claims 82 to 89, wherein the step of partially digesting the material is carried out in the presence of a surfactant.

91. A method according to claim 90, wherein the surfactant is sodium 3-[(2-methyl-2-undecyl-1,3-dioxolan-4-yl)methoxy]-1-propane-sulfonate.

92. A method according to any one of claims 82 to 89, wherein the step of partially digesting the material is carried out prior to applying the material to a carrier.

93. A method according to claim 92, wherein the digestion is carried out for from 1 to 24 hours.

94. A method according to any one of claims 82 to 89, wherein the step of partially digesting the material is carried out on a carrier.

95. A method according to claim 94, wherein the digestion is carried out for from 10 to 3600 seconds.

96. A method according to claim 93 wherein following digestion the material is applied to a carrier.

97. A method according to claim 96, further including the step of removing a portion of the liquid component of the material after application to the carrier, wherein the step of removing the portion of the liquid component is performed in a manner that does not destroy compounds within the material and partially dries the material.

98. A method according to claim 97, wherein the step of removing a portion of the liquid component is performed by a method selected from the group consisting of: applying an elevated temperature; reducing air pressure; passing a stream of gas over the surface of the applied material; allowing the applied material to sit at ambient temperature and pressure for a sufficient time for the liquid to be removed by evaporation; or a combination thereof.

99. A method according to claim 97 or 98, wherein at least 50% of the liquid component is removed.

100. A method according to claim 97 or 98, wherein at least 75% of the liquid component is removed.

101. A method according to claim 97 or 98, wherein at least 90% of the liquid component is removed.

102. A method according to claim 97 or 98, wherein removal of the liquid component continues until the material is at least substantially dry.

103. A method according to any one of claims 94 to 102 further including the step of applying a MALDI matrix over the material.

104. A method according to claim 103, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid; caffeic acid; 2,4,6-trihydroxyacetophenone; 3-hydroxypicolinic acid; Anthranilic acid; Nicotinic acid; Salicylamide and mixtures thereof.

105. A method according to any one of claims 82 to 104, wherein the step of comparing is performed manually by scanning the output of the MALDI-ToF MS and comparing it to known digestion fragments to determine the identity of a polypeptide in the material.

106. A method according to any one of claims 82 to 104, wherein the step of comparing is performed by computerised means.

107. A method according to claim 105, wherein output of the MALDI-ToF MS analysis is compared by computer means to a library of signature fragments to identify a polypeptide in the material.

108. A method of analysing a polypeptide variant in a material including the steps of:
partially digesting the material containing the polypeptide variant;
analysing the digested material by MALDI-ToF MS to determine digestion fragments; and
comparing the digestion fragments with the digestion fragments of non-variant polypeptides to identify the fragment containing the variation.

109. A method according to claim 108, wherein the material to be analysed includes a biological material or is derived from a biological material.

110. A method according to claim 109, wherein the biological material is selected from the group consisting of: blood, cerebrospinal fluid, urine, saliva, seminal fluid and sweat.

111. A method according to any one of claims 108 to 110, wherein the polypeptide is a haemoglobin polypeptide or a fragment or variant or a haemoglobin peptide containing a covalently bonded adduct thereof.

112. A method according to claim 111, wherein the haemoglobin polypeptide includes one or more haemoglobins selected from the group consisting of: α, β, γ, δ, ε and ζ haemoglobin.

113. A method according to any one of claims 108 to 112, wherein the step of partially digesting the material is carried out by contacting the material with a proteolytic agent.

114. A method according to claim 113, wherein the proteolytic agent is a protease.

115. A method according to claim 114, wherein the protease is selected from the group consisting of: trypsin and endoprotease Glu C.

116. A method according to any one of claims 108 to 115, wherein the step of partially digesting the material is carried out in the presence of a surfactant.

117. A method according to claim 116, wherein the surfactant is sodium 3-[(2-methyl-2-undecyl-1,3-dioxolan-4yl)methoxy]-1-propane-sulfonate.

118. A method according to any one of claims 108 to 117, wherein the step of partially digesting the material is carried out in solution prior to applying the material to a carrier.

119. A method according to claim 118, wherein the partial digestion is carried out for from 1 to 24 hours.

120. A method according to any one of claims 108 to 117, wherein the step of partially digesting the material is carried out on a carrier.

121. A method according to claim 120, wherein the partial digestion is carried out for from 10 to 3600 seconds.

122. A method according to claim 118 or 119 wherein following digestion the material is applied to a carrier.

123. A method according to claim 122 further including the step of removing a portion of the liquid component of the material after application to the carrier, wherein the step of removing the portion of the liquid component is performed in a manner that does not destroy compounds within the material and partially dries the material.

124. A method according to claim 123, wherein the step of removing a portion of the liquid component is performed by a method selected from the group consisting of: applying an elevated temperature; reducing air pressure; passing a stream of gas over the surface of the applied material; allowing the applied material to sit at ambient temperature and pressure for a sufficient time for the liquid to be removed by evaporation; or a combination thereof.

125. A method according to claim 123 or 124, wherein at least 50% of the liquid component is removed.

126. A method according to claim 123 or 124, wherein at least 75% of the liquid component is removed.

127. A method according to claim 123 or 124, wherein at least 90% of the liquid component is removed.

128. A method according to claim 123 or 124, wherein removal of the liquid component continues until the material is at least substantially dry.

129. A method according to any one of claims 120 to 128, further including the step of applying a MALDI matrix over the material.

130. A method according to claim 129, wherein the MALDI matrix is selected from the group consisting of: sinapinic acid; α-cyano-4-hydroxycinnamic acid; 2,5-dihydroxybenzoic acid; 2-(4-hydroxy phenylazo)benzoic acid; succinic acid, 2,6-Dihydroxyacetophenone; Ferulic acid; caffeic acid; 2,4,6-trihydroxyacetophenone; 3-hydroxypicolinic acid; Anthranilic acid; Nicotinic acid; Salicylamide and mixtures thereof.

131. A method according to any one of claims 108 to 130, wherein the step of comparing is performed manually by scanning the output of the MALDI-ToF MS and comparing it to known digestion fragments to determine the identity of a polypeptide variant in the material.

132. A method according to any one of claims 108 to 130, wherein the step of comparing is performed by computerised means.

133. A method according to claim 131, wherein output of the MALDI-ToF MS analysis is compared by computer means to a library of signature fragments to identify a polypeptide variant in the material.

134. A method of diagnosing a condition in a subject including the steps of: obtaining a material to be analysed from a subject;
analysing the material by MALDI-TOF MS to identify one or more
polypeptides within the material; and
determining from the presence or absence of a polypeptide within the material whether the subject has the condition.

135. A method according to claim 126, wherein the step of analysing the material involves analysing a polypeptide according to the method of any one of claims 64 to 81.

136. A method according to claim 134, wherein the condition to be diagnosed is either a condition that is diagnosed by either:
i. the absence of a polypeptide that would be present in material obtained from a non-afflicted subject; or
ii. the presence in the material of a polypeptide characteristic of the condition, said polypeptide not being present in a sample of a non-afflicted subject.

137. A method according to any one of claims 134 to 136, wherein the condition is a haemoglobinopathy.

138. A method according to claim 137, wherein the haemoglobinopathy is selected from the group consisting of: α-thalassemia (non-deletional, deletional, Hb H disease), β-thalassemia, δ-thalassemia, γ-thalassemia, hereditary persistence of fetal hemoglobin (HPFH), δβ-thalassemia, sickle cell disorder and other haemoglobin variant related disorders.
